SEQUENCE LISTING (105 sequences)

SEQ ID NO: 1 - protein, 247 aa, Mus musculus
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVIIQRKRI

SEQ ID NO: 2 - protein, 228 aa, Mus musculus
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVIIQRKRI

SEQ ID NO: 3 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 4 - protein, 254 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 5 - protein, 273 aa, Macaca fascicularis
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDATKRPVTTTKREVNSAI

SEQ ID NO: 6 - protein, 254 aa, Macaca fascicularis
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDATKRPVTTTKREVNSAI

SEQ ID NO: 7 - protein, 306 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 8 - protein, 269 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 9 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 10 - protein, 254 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 11 - protein, 309 aa, Mus musculus
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAE

SEQ ID NO: 12 - protein, 286 aa, Mus musculus
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAE

SEQ ID NO: 13 - protein, 323 aa, Homo sapiens
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 14 - protein, 300 aa, Homo sapiens
AYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 15 - protein, 309 aa, Mus musculus
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSSNTQGIENPGFETTPPFQGMPEAKTRPPLSYVAQRQPSESGRYLLSDPSTPLSPPGPGDVFFPSLDPVPDSPNSEAI

SEQ ID NO: 16 - protein, 277 aa, Mus musculus
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSSNTQGIENPGFETTPPFQGMPEAKTRPPLSYVAQRQPSESGRYLLSDPSTPLSPPGPGDVFFPSLDPVPDSPNSEAI

SEQ ID NO: 17 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 18 - protein, 279 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 19 - DNA, 663 bp, Mus musculus
atgctgctcctgctgccgatactgaacctgagcttacaacttcatcctgtagcagctttattcaccgtgacagcccctaaagaagtgtacaccgtagacgtcggcagcagtgtgagcctggagtgcgattttgaccgcagagaatgcactgaactggaagggataagagccagtttgcagaaggtagaaaatgatacgtctctgcaaagtgaaagagccaccctgctggaggagcagctgcccctgggaaaggctttgttccacatccctagtgtccaagtgagagattccgggcagtaccgttgcctggtcatctgcggggccgcctgggactacaagtacctgacggtgaaagtcaaagcttcttacatgaggatagacactaggatcctggaggttccaggtacaggggaggtgcagcttacctgccaggctagaggttatcccctagcagaagtgtcctggcaaaatgtcagtgttcctgccaacaccagccacatcaggacccccgaaggcctctaccaggtcaccagtgttctgcgcctcaagcctcagcctagcagaaacttcagctgcatgttctggaatgctcacatgaaggagctgacttcagccatcattgaccctctgagtcggatggaacccaaagtccccagaacgtgg

SEQ ID NO: 20 - protein, 221 aa, Mus musculus
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTW

SEQ ID NO: 21 - protein, 202 aa, Mus musculus
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTW

SEQ ID NO: 22 - DNA, 294 bp, Mus musculus
ttcaccgtgacagcccctaaagaagtgtacaccgtagacgtcggcagcagtgtgagcctggagtgcgattttgaccgcagagaatgcactgaactggaagggataagagccagtttgcagaaggtagaaaatgatacgtctctgcaaagtgaaagagccaccctgctggaggagcagctgcccctgggaaaggctttgttccacatccctagtgtccaagtgagagattccgggcagtaccgttgcctggtcatctgcggggccgcctgggactacaagtacctgacggtgaaa

SEQ ID NO: 23 - protein, 98 aa, Mus musculus
FTVTAPKEVYTVDVGSSVSLECDFDRRECTELEGIRASLQKVENDTSLQSERATLLEEQLPLGKALFHIPSVQVRDSGQYRCLVICGAAWDYKYLTVK

SEQ ID NO: 24 - DNA, 738 bp, Mus musculus
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaac

SEQ ID NO: 25 - protein, 246 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 26 - protein, 209 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 27 - DNA, 291 bp, Mus musculus
gttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttg

SEQ ID NO: 28 - protein, 97 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHL

SEQ ID NO: 29 - DNA, 732 bp, Mus musculus
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaag

SEQ ID NO: 30 - protein, 221 aa, Mus musculus
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWK

SEQ ID NO: 31 - protein, 221 aa, Mus musculus; identical to SEQ ID NO: 30

SEQ ID NO: 32 - DNA, 288 bp, Mus musculus
aatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaaca

SEQ ID NO: 33 - protein, 96 aa, Mus musculus
NGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLT

SEQ ID NO: 34 - DNA, 570 bp, Mus musculus
atgggtgtccccgcggtcccagaggccagcagcccgcgctggggaaccctgctccttgctattttcctggctgcatccagaggtctggtagcagccttcaaggtcaccactccatattctctctatgtgtgtcccgagggacagaatgccaccctcacctgcaggattctgggccccgtgtccaaagggcacgatgtgaccatctacaagacgtggtacctcagctcacgaggcgaggtccagatgtgcaaagaacaccggcccatacgcaacttcacattgcagcaccttcagcaccacggaagccacctgaaagccaacgccagccatgaccagccccagaagcatgggctagagctagcttctgaccaccacggtaacttctctatcaccctgcgcaatgtgaccccaagggacagcggcctctactgctgtctagtgatagaattaaaaaaccaccacccagaacaacggttctacgggtccatggagctacaggtacaggcaggcaaaggctcggggtccacatgcatggcgtctaatgagcaggacagtgacagcatcacggct

SEQ ID NO: 35 - protein, 190 aa, Mus musculus
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITA

SEQ ID NO: 36 - protein, 158 aa, Mus musculus
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITA

SEQ ID NO: 37 - DNA, 387 bp, Mus musculus
ttcaaggtcaccactccatattctctctatgtgtgtcccgagggacagaatgccaccctcacctgcaggattctgggccccgtgtccaaagggcacgatgtgaccatctacaagacgtggtacctcagctcacgaggcgaggtccagatgtgcaaagaacaccggcccatacgcaacttcacattgcagcaccttcagcaccacggaagccacctgaaagccaacgccagccatgaccagccccagaagcatgggctagagctagcttctgaccaccacggtaacttctctatcaccctgcgcaatgtgaccccaagggacagcggcctctactgctgtctagtgatagaattaaaaaaccaccacccagaacaacggttctacggg

SEQ ID NO: 38 - protein, 129 aa, Mus musculus
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYG

SEQ ID NO: 39 - DNA, 663 bp, Homo sapiens
atgatctttcttctcttgatgctgtctttggaattgcaacttcaccaaatcgcggccctctttactgtgaccgtgccaaaagaactgtatatcattgagcacgggtccaatgtgaccctcgaatgtaactttgacaccggcagccacgttaacctgggggccatcactgccagcttgcaaaaagttgaaaacgacacttcacctcaccgggagagggcaaccctcttggaggagcaactgccattggggaaggcctcctttcatatccctcaggtgcaggttcgggatgagggacagtaccagtgcattattatctacggcgtggcttgggattacaagtatctgaccctgaaggtgaaagcgtcctatcggaaaattaacactcacattcttaaggtgccagagacggacgaggtggaactgacatgccaagccaccggctacccgttggcagaggtcagctggcccaacgtgagcgtacctgctaacacttctcattctaggacacccgagggcctctaccaggttacatccgtgctccgcctcaaaccgcccccaggccggaattttagttgcgtgttttggaatacccacgtgcgagagctgactcttgcatctattgatctgcagtcccagatggagccacggactcatccaacttgg

SEQ ID NO: 40 - protein, 221 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 41 - protein, 202 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 42 - DNA, 294 bp, Homo sapiens
tttactgtgaccgtgccaaaagaactgtatatcattgagcacgggtccaatgtgaccctcgaatgtaactttgacaccggcagccacgttaacctgggggccatcactgccagcttgcaaaaagttgaaaacgacacttcacctcaccgggagagggcaaccctcttggaggagcaactgccattggggaaggcctcctttcatatccctcaggtgcaggttcgggatgagggacagtaccagtgcattattatctacggcgtggcttgggattacaagtatctgaccctgaag

SEQ ID NO: 43 - protein, 98 aa, Homo sapiens
FTVTVPKELYIIEHGSNVTLECNFDTGSHVNLGAITASLQKVENDTSPHRERATLLEEQLPLGKASFHIPQVQVRDEGQYQCIIIYGVAWDYKYLTLK

SEQ ID NO: 44 - DNA, 732 bp, Homo sapiens
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctc

SEQ ID NO: 45 - protein, 243 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNL

SEQ ID NO: 46 - protein, 209 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNL

SEQ ID NO: 47 - DNA, 303 bp, Homo sapiens
gttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacg

SEQ ID NO: 48 - protein, 101 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVT

SEQ ID NO: 49 - DNA, 741 bp, Homo sapiens
atgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtactt

SEQ ID NO: 50 - protein, 247 aa, Homo sapiens
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVL

SEQ ID NO: 51 - protein, 224 aa, Homo sapiens
AYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVL

SEQ ID NO: 52 - DNA, 294 bp, Homo sapiens
ccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttc

SEQ ID NO: 53 - protein, 98 aa, Homo sapiens
PCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANF

SEQ ID NO: 54 - DNA, 579 bp, Homo sapiens
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggct

SEQ ID NO: 55 - protein, 193 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITA

SEQ ID NO: 56 - protein, 161 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITA

SEQ ID NO: 57 - DNA, 390 bp, Homo sapiens
ttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggt

SEQ ID NO: 58 - protein, 130 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHG

SEQ ID NO: 59 - DNA, 663 bp, Macaca fascicularis
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttgg

SEQ ID NO: 60 - protein, 221 aa, Macaca fascicularis; identical to SEQ ID NO: 40

SEQ ID NO: 61 - protein, 202 aa, Macaca fascicularis; identical to SEQ ID NO: 41

SEQ ID NO: 62 - DNA, 294 bp, Macaca fascicularis
ttcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaa

SEQ ID NO: 63 - protein, 98 aa, Macaca fascicularis; identical to SEQ ID NO: 43

SEQ ID NO: 64 - protein, 221 aa, Artificial sequence
  Synthettic extracellular domain of human B7-DC with a K113S substitution and a signal peptide
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYSYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 65 - protein, 202 aa, Artificial sequence
  Synthettic extracellular domain of human B7-DC with a K113S substitution
LFTVTVPKELYIIEHGSNVTLECNFDTGSHVNLGAITASLQKVENDTSPHRERATLLEEQLPLGKASFHIPQVQVRDEGQYQCIIIYGVAWDYSYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 66 - protein, 98 aa, Artificial sequence
  synthetic IgV domain of human B7-DC with a K113S substitution
FTVTVPKELYIIEHGSNVTLECNFDTGSHVNLGAITASLQKVENDTSPHRERATLLEEQLPLGKASFHIPQVQVRDEGQYQCIIIYGVAWDYSYLTLK

SEQ ID NO: 67 - protein, 221 aa, Artificial sequence
  Synthetic extracellular domain of human B7-DC with a D111S substitution with signal sequence
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWSYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 68 - protein, 202 aa, Artificial sequence
  Synthetic extracellular domain of human B7-DC with a D111S substitution
LFTVTVPKELYIIEHGSNVTLECNFDTGSHVNLGAITASLQKVENDTSPHRERATLLEEQLPLGKASFHIPQVQVRDEGQYQCIIIYGVAWSYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 69 - protein, 98 aa, Artificial sequence
  Synthetic IgV domain of human B7-DC with a D111S substitution
FTVTVPKELYIIEHGSNVTLECNFDTGSHVNLGAITASLQKVENDTSPHRERATLLEEQLPLGKASFHIPQVQVRDEGQYQCIIIYGVAWSYKYLTLK

SEQ ID NO: 70 - DNA, 696 bp, Homo sapiens
gagcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaa

SEQ ID NO: 71 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 699 bp, Mus musculus
gagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaa

SEQ ID NO: 73 - protein, 233 aa, Mus musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 74 - protein, 4 aa, Artificial sequence
  Synthetic flexible peptide
GSGS

SEQ ID NO: 75 - protein, 4 aa, Artificial sequence
  Synthetic flexible peptide
GGGS

SEQ ID NO: 76 - protein, 15 aa, Artificial sequence
  Synthetic flexible peptide
GGGGSGGGGSGGGGS

SEQ ID NO: 77 - protein, 20 aa, Artificial sequence
  Synthetic flexible peptide
GGGGSGGGGSGGGGSGGGGS

SEQ ID NO: 78 - skipped

SEQ ID NO: 79 - DNA, 1365 bp, Artificial sequence
  Synthetic nucleotide encoding murine B7-DC fusion protein
atgctgctcctgctgccgatactgaacctgagcttacaacttcatcctgtagcagctttattcaccgtgacagcccctaaagaagtgtacaccgtagacgtcggcagcagtgtgagcctggagtgcgattttgaccgcagagaatgcactgaactggaagggataagagccagtttgcagaaggtagaaaatgatacgtctctgcaaagtgaaagagccaccctgctggaggagcagctgcccctgggaaaggctttgttccacatccctagtgtccaagtgagagattccgggcagtaccgttgcctggtcatctgcggggccgcctgggactacaagtacctgacggtgaaagtcaaagcttcttacatgaggatagacactaggatcctggaggttccaggtacaggggaggtgcagcttacctgccaggctagaggttatcccctagcagaagtgtcctggcaaaatgtcagtgttcctgccaacaccagccacatcaggacccccgaaggcctctaccaggtcaccagtgttctgcgcctcaagcctcagcctagcagaaacttcagctgcatgttctggaatgctcacatgaaggagctgacttcagccatcattgaccctctgagtcggatggaacccaaagtccccagaacgtgggagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaatga

SEQ ID NO: 80 - protein, 454 aa, Artificial sequence
  Synthetic murine B7-DC fusion protein with signal peptide
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 81 - protein, 435 aa, Artificial sequence
  Synthetic  murine B7-DC fusion protein
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 82 - DNA, 1362 bp, Artificial sequence
  Synthetic nucleotide encoding human B7-DC fusion protein
atgatctttcttctcttgatgctgtctttggaattgcaacttcaccaaatcgcggccctctttactgtgaccgtgccaaaagaactgtatatcattgagcacgggtccaatgtgaccctcgaatgtaactttgacaccggcagccacgttaacctgggggccatcactgccagcttgcaaaaagttgaaaacgacacttcacctcaccgggagagggcaaccctcttggaggagcaactgccattggggaaggcctcctttcatatccctcaggtgcaggttcgggatgagggacagtaccagtgcattattatctacggcgtggcttgggattacaagtatctgaccctgaaggtgaaagcgtcctatcggaaaattaacactcacattcttaaggtgccagagacggacgaggtggaactgacatgccaagccaccggctacccgttggcagaggtcagctggcccaacgtgagcgtacctgctaacacttctcattctaggacacccgagggcctctaccaggttacatccgtgctccgcctcaaaccgcccccaggccggaattttagttgcgtgttttggaatacccacgtgcgagagctgactcttgcatctattgatctgcagtcccagatggagccacggactcatccaacttgggaacctaaatcttgcgataaaactcatacctgtcccccttgcccagcccccgagcttctgggaggtcccagtgtgtttctgtttcccccaaaacctaaggacacacttatgatatcccgaacgccggaagtgacatgcgtggttgtggacgtctcacacgaagacccggaggtgaaattcaactggtacgttgacggagttgaggttcataacgctaagaccaagcccagagaggagcaatacaattccacctatcgagtggttagtgtactgaccgttttgcaccaagactggctgaatggaaaagaatacaagtgcaaagtatcaaacaaggctttgcctgcacccatcgagaagacaatttctaaagccaaagggcagcccagggaaccgcaggtgtacacactcccaccatcccgcgacgagctgacaaagaatcaagtatccctgacctgcctggtgaaaggcttttacccatctgacattgccgtggaatgggaatcaaatggacaacctgagaacaactacaaaaccactccacctgtgcttgacagcgacgggtcctttttcctgtacagtaagctcactgtcgataagtctcgctggcagcagggcaacgtcttttcatgtagtgtgatgcacgaagctctgcacaaccattacacccagaagtctctgtcactgagcccaggtaaatga

SEQ ID NO: 83 - protein, 453 aa, Artificial sequence
  Synthetic human B7-DC fusion protein with signal peptide
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 434 aa, Artificial sequence
  Synthetic  human B7-DC fusion protein
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85-87 - skipped (3 entries)

SEQ ID NO: 88 - DNA, 1437 bp, Artificial sequence
  Synthetic nucliec acid encoding murine B7-1 fusion protein
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacgagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaa

SEQ ID NO: 89 - protein, 479 aa, Artificial sequence
  Synthetic murine B7-1 fusion protein with signal peptide
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 90 - protein, 442 aa, Artificial sequence
  Synthetic murine B7-1 fusion protein
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 91 - DNA, 1425 bp, Artificial sequence
  synthetic nucleic acid encoding  human B7-1 fusion protein
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctggagcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaa

SEQ ID NO: 92 - protein, 475 aa, Artificial sequence
  Synthetic human B7-1 fusion protein with signal peptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 441 aa, Artificial sequence
  Synthetic human B7-1 fusion protein
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - DNA, 1431 bp, Artificial sequence
  Synthetic nucleic acid encoding  murine B7-2 fusion protein
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaa

SEQ ID NO: 95 - protein, 477 aa, Artificial sequence
  Synthetic murine B7-2 fusion protein with signal sequence
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 96 - protein, 454 aa, Artificial sequence
  Synthetic murine B7-2 fusion protein
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 97 - DNA, 1437 bp, Artificial sequence
  Synthetic nucleotide encoding  human B7-2 fusion protein
atgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttgagcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaa

SEQ ID NO: 98 - protein, 479 aa, Artificial sequence
  Synthetic human B7-2 fusion protein with signal peptide
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 456 aa, Artificial sequence
  Synthetic human B7-2 fusion protein
AYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1269 bp, Artificial sequence
  Synthetic nucleotide encoding murine B7-H5 fusion protein
atgggtgtccccgcggtcccagaggccagcagcccgcgctggggaaccctgctccttgctattttcctggctgcatccagaggtctggtagcagccttcaaggtcaccactccatattctctctatgtgtgtcccgagggacagaatgccaccctcacctgcaggattctgggccccgtgtccaaagggcacgatgtgaccatctacaagacgtggtacctcagctcacgaggcgaggtccagatgtgcaaagaacaccggcccatacgcaacttcacattgcagcaccttcagcaccacggaagccacctgaaagccaacgccagccatgaccagccccagaagcatgggctagagctagcttctgaccaccacggtaacttctctatcaccctgcgcaatgtgaccccaagggacagcggcctctactgctgtctagtgatagaattaaaaaaccaccacccagaacaacggttctacgggtccatggagctacaggtacaggcaggcaaaggctcggggtccacatgcatggcgtctaatgagcaggacagtgacagcatcacggctgagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaa

SEQ ID NO: 101 - protein, 423 aa, Artificial sequence
  Synthetic murine B7-H5 fusion protein with signal peptide
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 102 - protein, 391 aa, Artificial sequence
  Synthetic murine B7-H5 fusion protein
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 103 - DNA, 1275 bp, Artificial sequence
  Synthetic nucleotide encoding  human B7-H5 fusion protein
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgagcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaa

SEQ ID NO: 104 - protein, 425 aa, Artificial sequence
  Synthetic human B7-H5 fusion protein with signal peptide
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 393 aa, Artificial sequence
  Synthetic human B7-H5 fusion protein
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0223188 A1
(43) Pub. Date: Sep. 15, 2011

(54) TARGETED COSTIMULATORY POLYPEPTIDES AND METHODS OF USE TO TREAT CANCER

(76) Inventor: Solomon Langermann, Baltimore, MD (US)

(21) Appl. No.: 13/060,909

(22) PCT Filed: Aug. 25, 2009
(86) PCT No.: PCT/US09/54969

§ 371 Date: May 19, 2011

(30) Foreign Application Priority Data
Aug. 25, 2008 (US) 61/091502
Aug. 25, 2008 (US) 61091694
Aug. 25, 2008 (US) 61091705
Aug. 25, 2008 (US) 61091709
Jan. 5, 2009 (US) 61/142548
Apr. 1, 2009 (US) 61165652

(51) Int. Cl. A61K 39/39; C07K 19/00; C07H 21/00; C12N 15/63; C12N 1/00; C12N 5/10; C12N 5/0783; A61K 31/7088; A61P 37/04; A61P 35/00

(52) U.S. Cl. 424/192.1; 530/409; 530/351; 530/391.7; 536/23.4; 435/320.1; 435/243; 435/325; 435/375; 514/44 R

(57) ABSTRACT

Compositions are provided that are targeted to tumors or tumor-associated neovasculature and enhance the function of tumor-infiltrating T cells. The compositions include fusion proteins that contain a T cell binding domain and a tumor/tumor-associated neovasculature targeting domain. The fusion proteins optionally contain a peptide/polypeptide linker domain and a domain that mediates dimerization or multimerization. The T cell binding domain can be a costimulatory molecule. Methods for using the fusion proteins to enhance an immune response are provided. Therapeutic uses for the disclosed compositions include the induction of tumor immunity.

### CROSS-REFERENCE TO RELATED APPLICATIONS

This application claims priority to and benefit of U.S. Provisional Application No. 61/091,502, filed on Aug. 25, 2008, U.S. Provisional Application No. 61/091,694, filed on Aug. 25, 2008, U.S. Provisional Application No. 61/091,709, filed on Aug. 25, 2008, U.S. Provisional Application No. 61/091,705, filed on Aug. 25, 2008, and U.S. Provisional Application No. 61/142,548, filed on Jan. 5, 2009, and U.S. Provisional Application No. 61/165,652 filed on Apr. 1, 2009, and where permissible are incorporated by reference in their entireties.

### FIELD OF THE INVENTION

This invention relates to compositions and methods for modulating T cell activation, in particular to compositions and methods for enhancing T cell activation in tumor microenvironments and in tissues involved in immune cell activation.

### BACKGROUND OF THE INVENTION

Cancer has an enormous physiological and economic impact. For example a total of 1,437,180 new cancer cases and 565,650 deaths from cancer are projected to occur in the United States in 2008 (Jenial, A., Cancer J. Clin., 58:71-96 (2008)). The National Institutes of Health estimate overall costs of cancer in 2007 at $219.2 billion: $89.0 billion for direct medical costs (total of all health expenditures); $18.2 billion for indirect morbidity costs (cost of lost productivity due to illness); and $112.0 billion for indirect mortality costs (cost of lost productivity due to premature death). Although there are several methods for treating cancer, each method has its own degree of effectiveness as well as side-effects. Typical methods for treating cancer include surgery, chemotherapy, radiation, and immunotherapy.

Stimulating the patients own immune response to target tumor cells is an attractive option for cancer therapy and many studies have demonstrated effectiveness of immunotherapy using tumor antigens to induce the immune response. However, induction of an immune response and the effective eradication of cancer often do not correlate in cancer immunotherapy trials (Cormier, et al., Cancer J. Sci. Am., 3(1):37-44 (1997); Nestle, et al., Nat. Med, 4(3):328-332 (1998); Rosenberg, Nature, 411(6835):380-384 (2001)). Thus, despite primary anti-tumor immune responses in many cases, functional, effector anti-tumor T cell responses are often weak at best.

An antigen specific T cell response is mediated by two signals: 1) engagement of the TCR with antigenic peptide presented in the context of MHC (signal 1), and 2) a second antigen-independent signal delivered by contact between different receptor/ligand pairs (signal 2). This “second signal” is critical in determining the type of T cell response (activation vs inhibition) as well as the strength and duration of that response, and is regulated by both positive and negative signals from costimulatory molecules, such as the B7 family of proteins. The most extensively characterized T cell costimulatory pathway is B7-CD28, in which B7-1 (CD80) and B7-2 (CD86) each can engage the stimulatory CD28 receptor and the inhibitory CTLA-4 (CD 152) receptor. In conjunction with signaling through the T cell receptor, CD28 ligation increases antigen-specific proliferation of T cells, enhances production of cytokines, stimulates differentiation and effector function, and promotes survival of T cells (Lenshow, et al., Annu. Rev. Immunol, 14:233-258 (1996); Chambers and Allison, Curr. Opin. Immunol, 9:396-404 (1997); and Rathmell and Thompson, Annu. Rev. Immunol., 17:781-828 (1999)). In contrast, signaling through CTLA-4 is thought to deliver a negative signal that inhibits T cell proliferation, IL-2 production, and cell cycle progression (Krunimel and Allison, J. Exp. Med, 183:2533-2540 (1996); and Walunas, et al., J. Exp. Med., 183:2541-2550 (1996)). Other members of the 137 family include 137-H1 (Dong, et al., Nature Med., 5:1365-1369 (1999); and Freeman, et al., J. Exp. Med., 192:1-9 (2000)), B7-DC (also Tseng, et al., J. Exp. Med., 193:839-846 (2001); and Latchman, et al., Nature Immunol., 2:261-268 (2001)), B7-H2 (Wang, et al., Blood, 96:2808-2813 (2000); Swallow, et al., Immunity, 11:423-432 (1999); and Yoshinaga, et al., Nature, 402:827-832 (1999)), B7-H3 (Chapoval, et al., Nature Immunol., 2:269-274 (2001)) and B7-H4 (Choi, et al., J. Immunol., 171:4650-4654 (2003); Sica, et al., Immunity, 18:849-861 (2003); Prasad, et al., Immunity, 18:863-873 (2003); and Zang, et al., Proc. Natl. Acad. Sci. U.S.A., 100:10388-10392 (2003)). B7-H1 (also known as PD-L1) and B7-DC (also known as PD-L2) are ligands for PD-1, B7-H2 is a ligand for ICOS, and B7-H3 and B7-H4 remain orphan ligands at this time (Dong, et al., Immunol. Res., 28:39-48 (2003)).

Certain molecules such as those of the B7 family can enhance effector immune responses to tumor/tumor antigens. Exogenous delivery of costimulatory molecules that enhance T cell response in vivo is therefore thought to be a practical way to augment the immune response to tumors. However, reaching an effective level of costimulatory molecules in vivo may require a large amount of recombinant protein. Systemic delivery of costimulatory molecules in vivo can also result in non-specific immune activation that can be harmful to the host.

Therefore, it is an object of the invention to provide T cell costimulatory compositions that enhance T cell responses and are targeted to tumors or tumor-associated neovasculature and methods for their use.

It is another object of the invention to provide costimulatory compositions that enhance T cell responses and can concentrate inside tumors in vivo and augment the function of tumor-infiltrating T cells.

It is another object of the invention to provide costimulatory molecule compositions that enhance T cell responses and reduce the amount of costimulatory molecule necessary to achieve effective anti-tumor T cell responses in vivo.

It is another object of the invention to provide costimulatory molecule compositions that enhance T cell responses and reduce non-specific immune activation in a host.

### SUMMARY OF THE INVENTION

Compositions are provided that are targeted to tumors or tumor-associated neovasculature and enhance the function of tumor-infiltrating T cells. The compositions include fusion proteins that contain a T cell binding domain, a tumor/tumor-associated neovasculature targeting domain and optionally a linker domain. The linker is preferably a peptide/polypeptide.

In one embodiment, the T cell binding domain is a costimulatory molecule or a variant and/or fragment thereof that binds to and activates a receptor on T cells, resulting in enhanced T cell responses. Representatives of such receptor agonists include members of the B7 family, including, but not limited to, B7-1, B7-2, and B7-H5. Useful fragments of said costimulatory molecules include soluble fragments, including the extracellular domain, or fragments thereof, including the IgV and/or IgC domains. Agonistic single polypeptide antibodies or fragments thereof that bind to and activate costimulatory receptors and lead to enhanced T cell responses are also useful T cell activating domains.

The tumor/tumor-associated neovasculature targeting domain is a domain that binds to an antigen, receptor or ligand that is specific for tumors or tumor-associated neovasculature, or is overexpressed in tumors or tumor-associated neovasculature as compared to normal tissue. Suitable antigens that can be targeted include, but are not limited to, tumor-specific and tumor-associated antigens and antigens overexpressed on tumor-associated neovasculature including, but not limited to, VEGF/KDR, Tie2, vascular cell adhesion molecule (VCAM), endoglin and α₅β₃ integrin/vitronectin. Suitable tumor/tumor-associated neovasculature targeting domains include, but are not limited to, ligands, receptors, single polypeptide antibodies and immunoglobulin Fc domains.

The peptide/polypeptide linker domain can be any flexible peptide or polypeptide at least 2 amino acids in length that separates the T cell binding domain and the tumor/tumor-associated neovasculature targeting domain and provides increased rotational freedom between these two domains. Suitable polypeptides include the hinge region of immunoglobulins alone, or in combination with either immunoglobulin Fc regions or the C_{H}1 or C_{L} regions.

The fusion proteins can also contain dimerization or multimerization domains that can either be separate domains or can be contained within the T cell binding domain, the tumor/tumor-associated neovasculature targeting domain or the peptide/polypeptide linker domain. Preferred dimerization domains contain at least one cysteine that is capable of forming an intermolecular disulfide bond. Other suitable dimerization/multimerization domains are provided.

The fusion proteins can be dimerized or multimerized to form homodimers, heterodimers, homomultimers or heteromultimers. Dimerization or multimerization can occur either through dimerization/multimerization domains, or can be the result of chemical crosslinking. Dimerization/multimerization partners can be arranged either in parallel or antiparallel orientations.

Isolated nucleic acids molecules encoding the disclosed fusion proteins, vectors and host cells, and pharmaceutical and immunogenic compositions containing the fusion proteins are also provided. Immunogenic compositions contain antigens, a source of fusion proteins and, optionally, additional adjuvants.

Methods for using the fusion proteins to increase T cell responses and block inhibition of T cell activation, or to reverse T cell exhaustion and anergy, are also provided. Therapeutic uses for the disclosed compositions include the induction of tumor immunity. The tumor or tumor-associated neovasculature binding domains function to effectively target the fusion proteins to the tumor microenvironment, where they can specifically enhance the activity of tumor-infiltrating T cells through their T cell binding domains. The ability of the compositions to concentrate in tumors reduces the amount of costimulatory molecule that is necessary to administer in vivo to achieve an effective amount, and thereby reduces the risk of non-specific activation of the immune system. Fusion proteins can be administered as monomers, dimers or multimers. In one embodiment, fusion proteins are administered as dimers or multimers that have increased valency for T cell and/or tumor/tumor-associated neovasculature binding determinants.

Also provided are methods for administering fusion protens in combination with other tumor therapies or as part of a prophylactic or therapeutic vaccine composition.

### BRIEF DESCRIPTION OF THE DRAWINGS

FIG. 1 is a diagram of an exemplary dosing regimen for the P815 tumor model.

FIGS. 2A-C is a line graphs of tumor volumes plotted as a function of time and treatment: A) vehicle control, B) mouse IgG control, and C) murine B7-DC-Ig.

FIGS. 3A and B are line graphs of tumor growth (mm³) versus days post tumor inoculation in mice given 100 mg/kg cyclophosphamide (CTX or Cytoxan®) alone (FIG. 3A) and mice given the combination of CTX and dimeric murine B7-DC-Ig (FIG. 3B). The combination of B7-DC-Ig and CTX resulted in eradication of established CT26 tumors (colon carcinoma) in mice. Each line in each graph represents one mouse. Black arrow stands for B7-DC-Ig administration. FIG. 3C is a line graph of average average tumor volume versus days post tumor implanation in mice given 100 mg/kg CTX (--) or the combination of CTX and dimeric murine B7-DC-Ig (-▴-).

FIG. 4 shows the results of experiments wherein the combination of CTX and dimeric murine B7-DC-Ig eradicated established CT26 tumors (colon carcinoma) in mice and protected against re-challenge with CT26. Mice that were treated with CTX and B7-DC-Ig and found to be free of tumor growth on day 44 following tumor inoculation were rechallenged with tumors. The mice were later rechallenged again on on Day 70. None of the mice displayed tumor growth by day 100.

FIG. 5 shows CTX and 137-DC-Ig treatment resulted in generation of tumor specific memory CTL. Mice eradicated established CT26 subcutenous tumors post CTX and B7-DC-Ig treatment were re-challenged with CT26 cells. Seven days later, splenocytes were isolated and pulsed with either ovalbumin, an irrelevant peptide, or AH1, a CT26 specific peptide. Cells were stained with anti-CD8 antibody first followed by intracellular staining with anti-IFNγ antibody prior to FACS analysis.

FIGS. 6A and B show the results of experiments wherein Balb/C mice at age of 9 to 11 weeks of age were implanted with 1×10⁵ CT26 cells subcutaneously. On Day 9, mice were injected with 100 mg/kg of CTX, IP. Twenty four hours later, on Day 10, mice were treated with 100 ug of 137-DC-Ig. There were 5 groups: naïve mice that did not receive any tumor cells, vehicle injected, CTX alone, CTX B7-DC-Ig or B7-DC-Ig alone. Two naïve mice and 4 mice from other groups were removed from the study on Day 11 (2 days post CTX) and Day 16 (7 days post CTX) for T cell analysis. FIG. 6A shows on Day 11, 2 days post CTX injection, Treg in the spleen of the mice with CTX treatment was significantly lower than the one in the mice with tumor implantation and injected with vehicle. FIG. 6B shows that on Day 16, 7 days post CTX and 6 days post B7-DC-Ig treatment, B7-DC-Ig significantly lowered the CD4+ T cells expressing high PD-1. This was observed in both the B7-DC-Ig treated and CTX+B7-DC-Ig treated mice. Mice implanted with tumor cells intended to have more PD-1+/CD4+ T cells in the draining LN compared with naïve mice.

FIG. 7 is a line graph of survival (%) versus days post tumor implantation in mice administered with the combination of CTX and B7-DC-Ig (-▴-), CTX alone (dashed line), or B7-DC-Ig alone (solid line). SP-1 cells were isolated from mouse lungs that were metastasized from. TRAMP prostate tumor cell injection. B10.D2 mice were first injected with 3×10⁵ SP-1 cells via tail vein injection. On Day 5, 12 and 19, mice were injected with 50 mg/kg of CTX where was indicated. On Day 6, 13 and 20, mice were administered with 5 mg/kg of B7-DC-Ig were it was indicated. Here, “NT” refers to “not treated”.

FIG. 8 is line graph of overall survival (%) versus days post tumor implantation in Balb/C mice at age of 11-13 weeks given isolated hepatic metastases using a hemispleen injection technique. The spleens of anesthetized mice were divided into two halves and the halves were clipped. CT26 cells (1E05) were injected into one hemispleen, and after 30 seconds, that hemispleen was resected and the splenic draining vein was clipped. On Day 10, mice received 1 injection of CTX at 50 mg/kg, IP. Twenty four hours later, on Day 11, mice were treated with recombinant Listeria carrying AH1 peptide, an immunodominant epitope of CT26, at 0.1×LD50 (1×107 CFU), then on Day 14 and 17. Mice were also treated with B7-DC-Ig on Day 11 and then on Day 18. Mouse overall survival was monitored.

### DETAILED DESCRIPTION OF THE INVENTION

### I. Definitions

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the term “polypeptide” refers to a chain of amino acids of any length, regardless of modification (e.g., phosphorylation or glycosylation).

As used herein, a “costimulatory polypeptide” or “costimulatory molecule” is a polypeptide that, upon interaction with a cell-surface molecule on T cells, modulates the activity of the T cell. Costimulatory signaling can inhibit T cell function or enhance T cell function depending on which T cell receptor is activated or blocked.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

“Operably linked” refers to an arrangement of elements wherein the components so described are configured so as to perform their usual or intended function. Thus, two different polypeptides operably linked together retain their respective biological functions while physically linked together.

As used herein, “valency” refers to the number of binding sites available per molecule.

As used herein, the term “host cell” refers to prokaryotic and eukaryotic cells into which a recombinant expression vector can be introduced.

As used herein, “transformed” and “transfected” encompass the introduction of a nucleic acid (e.g. a vector) into a cell by a number of techniques known in the art.

As used herein, the term “antibody” is meant to include both intact molecules as well as fragments thereof that include the antigen-binding site. These include Fab and F(ab′)₂ fragments which lack the Fc fragment of an intact antibody.

The terms “individual”, “host”, “subject”, and “patient” are used interchangeably herein, and refer to a mammal, including, but not limited to, humans, rodents such as mice and rats, and other laboratory animals.

### II. Fusion Proteins

The compositions disclosed herein are fusion proteins that contain a costimulatory polypeptide domain and a domain that is an antigen-binding domain that targets the fusion protein to tumor cells, tumor cell-associated neovasculature, or to tissues involved in T cell activation. The costimulatory polypeptide can either bind to a T cell receptor and enhance a T cell response

The fusion proteins also optionally contain a peptide or polypeptide linker domain that separates the costimulatory polypeptide domain from the antigen-binding domain.

Fusion proteins disclosed herein are of formula I:

N—R₁—R₂—R₃—C

wherein “N” represents the N-terminus of the fusion protein, “C” represents the C-terminus of the fusion protein, “R₁” is a costimulatory polypeptide domain or a antigen-binding targeting domain, “R₂” is a peptide/polypeptide linker domain, and “R₃” is a costimulatory polypeptide domain or a antigen-binding targeting domain, wherein “R₃” is a costimulatory polypeptide domain when “R₁” is a antigen-binding targeting domain, and “R₃” is a antigen-binding targeting domain when “R₁” is a costimulatory polypeptide domain. In a preferred embodiment, “R₁” is a costimulatory polypeptide domain and “R₃” is a antigen-binding targeting domain.

Optionally, the fusion proteins additionally contain a domain that functions to dimerize or multimerize two or more fusion proteins. The domain that functions to dimerize or multimerize the fusion proteins can either be a separate domain, or alternatively can be contained within one of one of the other domains (costimulatory polypeptide domain, antigen-binding targeting domain, or peptide/polypeptide linker domain) of the fusion protein.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

The modular nature of the fusion proteins and their ability to dimerize or multimerize in different combinations provides a wealth of options for targeting molecules that function to costimulate T cells to the tumor cell microenvironment or to immune regulatory tissues.

A. Costimulatory Molecules that Enhance Immune Responses

The fusion proteins disclosed herein include costimulatory polypeptides of the B7 family, or biologically active fragments and/or variants thereof. Representative co-stimulatory polypeptides include, but are not limited to B7-1, B7-2, and B7-H5. These costimulatory polypeptides can activate T cell function. In a preferred embodiment, the extracellular domain or a biologically active fragment thereof is used as a T cell costimulatory polypeptide.

It has been shown that B7-DC binds to PD-1, a distant member of the CD28 receptor family that is inducibly expressed on activated T cells, B cells, natural killer (NK) cells, monocytes, DC, and macrophages (Keir, et al Curr. Opin. Immunol. 19:309-314 (2007)). The phenotypes of PD-1−/− mice provide direct evidence for PD-1 being a negative regulator of immune responses in vivo. In the absence of PD-1, mice on the C57BL/6 background slowly develop a lupus-like glomerulonephritis and progressive arthritis (Nishimura, et al., Immunity, 11:141-151 (1999)). PD-1−/− mice on the BALB/c background rapidly develop a fatal autoimmune dilated cardiomyopathy (Nishimura, et al., Science. 291:319-322 (2001)). Therefore, by binding to PD-1, B7-DC is a costimulatory molecule that inhbits T cell function. However, substantial evidence indicates that B7-DC can function to costimulate activate T cell responses. In the presence of suboptimal TCR signals, B7-DC causes increased proliferation and production of cytokines in vitro (Tseng, et al., J. Exp. Med. 193:839-846 (2001)). On the other hand, in vitro studies indicate a negative regulatory role for B7-DC in T cell responses. These seemingly contradictory data are best interpreted by expression of additional receptors for B7-DC on T cells other than PD-1. Therefore, in certain circumstances, B7-DC acts as a costimulatory polypeptide that can activate T cell function.

The B7 costimulatory polypeptide may be of any species of origin. In one embodiment, the costimulatory polypeptide is from a mammalian species. In a preferred embodiment, the costimulatory polypeptide is of murine or human or non-human primate origin. Useful human B7 costimulatory polypeptides have at least about 80, 85, 90, 95 or 100% sequence identity to the B7-DC polypeptide encoded by the nucleic acid having GenBank Accession Number NM_{—}025239; the B7-1 polypeptide encoded by the nucleic acid having GenBank Accession Number NM_{—}005191; the B7-2 polypeptide encoded by the nucleic acid having GenBank Accession Number U04343 or; the B7-H5 polypeptide encoded by the nucleic acid having GenBank Accession Number NP_{—}071436. B7-H5 is also disclosed in PCT Publication No. WO 2006/012232.

1. Fragments of B7 Costimulatory Polypeptides

The B7 polypeptides disclosed herein can be full-length polypeptides, or can be a fragment of a full length B7 polypeptide. As used herein, a fragment of B7 polypeptides refers to any subset of the polypeptide that is a shorter polypeptide of the full length protein. In certain embodiments, the fragments retain the ability to co-stimulate T cells. Fragments of B7 costimulatory molecules may be useful to reduce the size of the fusion protein in order to facilitate the simultaneous association of the costimulatory molecule with a costimulatory receptor on T cells in concert with CD3/T cell receptor engagement during formation of immune synapses.

Useful fragments are those that retain the ability to bind to their natural ligands. A costimulatory polypeptide that is a fragment of full-length costimulatory polypeptide typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand(s) as compared to the full-length costimulatory polypeptide.

One embodiment provides B7 polypeptide fragments that retain the ability to costimulate T cells. A 137 polypeptide that is a fragment of a full-length B7 polypeptide typically has at least 20 percent, 30 percent, 40 percent, 50 percent, 60 percent, 70 percent, 80 percent, 90 percent, 95 percent, 98 percent, 99 percent, 100 percent, or even more than 100 percent of the costimulatory activity of the full-length B7 polypeptide.

Human and mouse and non-human primate B7 proteins contain short intracytoplasmic domains, a single transmembrane domain and an extracellular domain. The extracellular domain typically contains two Ig domains; a membrane proximal IgC domain and a membrane distal IgV domain. Useful fragments of B7 costimulatory polypeptides include soluble fragments. Soluble B7 costimulatory polypeptide fragments are fragments of B7 costimulatory polypeptides that may be shed, secreted or otherwise extracted from the producing cells. Soluble fragments of B7 costimulatory polypeptides include some or all of the extracellular domain of the B7 costimulatory polypeptide, and lack some or all of the intracellular and/or transmembrane domains. In one embodiment, B7 costimulatory polypeptide fragments include the entire extracellular domain of the B7 costimulatory B7 costimulatory polypeptide. In other embodiments, the soluble fragments of B7 costimulatory polypeptides include fragments of the extracellular domain that retain B7 costimulatory biological activity. It will be appreciated that the extracellular domain can include 1, 2, 3, 4, or 5 amino acids from the transmembrane domain. Alternatively, the extracellular domain can have 1, 2, 3, 4, or 5 amino acids removed from the C-terminus, N-terminus, or both.

Generally, the B7 costimulatory polypeptides or fragments thereof are expressed from nucleic acids that include sequences that encode a signal sequence. The signal sequence is generally cleaved from the immature polypeptide to produce the mature polypeptide lacking the signal sequence. It will be appreciated that the signal sequence of B7 costimulatory polypeptides can be replaced by the signal sequence of another polypeptide using standard molecule biology techniques to affect the expression levels, secretion, solubility, or other property of the polypeptide. The signal sequence that is used to replace the B7 costimulatory polypeptide signal sequence can be any known in the art.

B7-DC

Murine B7-DC polypeptides can have at least 80%, 85%, 90%, 95%, 99% or 100% sequence identity to:

| (SEQ ID NO: 1) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT WPLHVFIPAC TIALIFLAIV | 240 |
| IIQRKRI | 247 |
| or | |
| (SEQ ID NO: 2) | |
| LFTVTAPKEV YTVDVGSSVS LECDFDRREC TELEGTRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TWPLHVFIPA CTIALIFLAI VIIQRKRI. | 228 |

Human B7-DC polypeptides can have at least 80%, 85%, 90%, 95%, 99% or 100% sequence identity to:

| (SEQ ID NO: 3) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WLLHIFIPFC IIAFIFIATV | 240 |
| IALRKQLCQK LYSSKDTTKR PVTTTKREVN SAI | 273 |
| or | |
| (SEQ ID NO: 4) | |
| LFTVTVPKEL YIIEHGSNVT LECNFDTGSH VNLGAITASL QKVEHDTSPH RERATLLEEQ | 60 |
| LPLGKASPHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TWLLHIFIPF CIIAFIFIAT VIALRKQLCQ KLYSSKDTTK | 240 |
| RPVTTTKREV NSAI. | 254 |

Non-human primate (Cynomolgus) B7-DC polypeptides can have at least 80%, 85%, 90%, 95%, 99% or 100% sequence identity to:

| (SEQ ID NO: 5) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFNNTHVR ELTLASIDLQ SQMEPRTHPT WLLHIFIPSC IIAFIFIATV | 240 |
| IALRKQLCQK LYSSKDATKR PVTTTKREVN SAI | 273 |
| or | |
| (SEQ ID NO: 6) | |
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVENNTHV | 180 |
| RELTLASIDL QSQMEPRTHP TWLLHIFIPS CIIAFIFIAT VIALRKQLCQ KLYSSKDATK | 240 |
| RPVTTTKREV NSAI | 254 |

It will be appreciated that SEQ ID NOs: 1, 3 and 5 each contain a signal peptide.

B7-1

Murine B7-1 polypeptides can have at least 80%, 85%, 90%, 95%, 99% or 100% sequence identity to:

| (SEQ ID NO: 7) | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLTILGLV LSDRGTYSCV | 120 |
| VQKKERGTYE VKHLALVKLS IKADFSTPNI TESGNPSADT KRITCFASGG FPKPRFSWLE | 180 |
| NGRELPGINT TISQDPESEL YTISSQLDFN TTRNHTIKCL IKYGDAHVSE DFTWEKPPED | 240 |
| PPDSKNTLVL FGAGFGAVIT VVVIVVIIKC FCKHRSCFRR NEASRETNNS LTFGPEEALA | 300 |
| EQTVFL | 306 |
| or | |
| (SEQ ID NO: 8) | |
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHLAIV KLSIKADFST PNITESGNPS | 120 |
| ADTKRITCFA SGGFPKPRFS WLENGRELPG INTTISQDPE SELYTISSQL DFNTTRNHTI | 180 |
| KCLIKYGDAH VSEDFTWEKP PEDPPDSKNT LVLFGAGFGA VITVVVIVVI IKCFCKHRSC | 240 |
| FRRNEASRET NNSLTFGPEE ALAEQTVFL. | 269 |

Human B7-1 polypeptides can have at least 80%, 85%, 90%, 95%, 99% or 100% sequence identity to:

| (SEQ ID NO: 9) | |
|---|---|
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNLLPSWAIT LISVNGIFVI CCLTYCFAPR CRERRRNERL RRESVRPV | 288 |
| or | |
| (SEQ ID NO: 10) | |
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADEPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNLL PSWAITLISV NGIFVICCLT YCFAPRCRER | 240 |
| RRNERLRRES VRPV. | 254 |

It will be appreciated that SEQ ID NOs: 7 and 9 each contain a signal peptide.

B7-2

Murine B7-2 polypeptides can have at least 80%, 85%, 90%, 95%, 99% or 100% sequence identity to:

| (SEQ ID NO: 11) | |
|---|---|
| MDPRCTMGLA ILIFVTVLLI SDAVSVETQA YFNGTAYLPC PFTKAQNISL SELVVFWQDQ | 60 |
| QKLVLYEHYL GTEKLDSVNA KYLGRTSFDR NNWTLRLHNV QIKDMGSYDC FIQKKPPTGS | 120 |
| IILQQTLTEL SVIANFSEPE IKLAQNVTGN SGINLTCTSK QGHPKPKKMY FLITNSTNEY | 180 |
| GDNMQISQDN VTELFSISNS LSLSFPDGVW HMTVVCVLET ESMKISSKPL NFTQEEPSPQ | 240 |
| TYWKEITASV TVALLLVMLL IIVCHKKPNQ PSRPSNTASK LERDSNADRE TINLKELEPQ | 300 |
| IASAKPNAE | 309 |
| or | |
| (SEQ ID NO: 12) | |
| VSVETQAYEN GTAYLPCPFT KAQNISLSEL VVFWQDQQKL VLYEHYLGTE KLDSVNAKYL | 60 |
| GRTSFDRNNW TLRLHNVQIK DMGSYDCFIQ KKPPTGSIIL QQTLTELSVI ANFSEPEIKL | 120 |
| AQNVTGNSGI NLTCTSKQGH PKPKKMYFLI TNSTNEYGDN MQISQDNVTE LFSISNSLSL | 180 |
| SFPDGVWHMT VVCVLETESM KISSKPLNFT QEEPSPQTYW KEITASVTVA LLLVMLLIIV | 240 |
| CHKKPNQPSR PSNTASKLER DSNADRETIN LKELEPQIAS AKPNAE. | 286 |

Human B7-2 polypeptides can have at least 80%, 85%, 90%, 95%, 99% or 100% sequence identity to:

| (SEQ ID NO: 13) | |
|---|---|
| MGLSNILFVM AFLLSGAAPL KIQAYFNETA DLPCQFANSQ NQSLSELVVF WQDQENLVLN | 60 |
| EVYLGKEKFD SVHSKYMGRT SFDSDSWTLR LHNLQIKDKG LYQCIIHHKK PTGMIRIHQM | 120 |
| NSELSVLANF SQPEIVPISN ITENVYINLT CSSIHGYPEP KKMSVLLRTK NSTIEYDGIM | 180 |
| QKSQDNVTEL YDVSISLSVS FPDVTSNMTI FCILETDKTR LLSSPFSIEL EDPQPPPDHI | 240 |
| PWITAVLPTV IICVMVFCLI LWKWKKKKRP RNSYKCGTNT MEREESEQTK KREKIHIPER | 300 |
| SDEAQRVFKS SKTSSCDKSD TCF | 323 |
| or | |
| (SEQ ID NO: 14) | |
| AYFNETADLP CQFANSQNQS LSELVVFWQD QENLVLNEVY LGKEKFDSVH SKYMGRTSFD | 60 |
| SDSWTLRLHN LQIKDKGLYQ CIIHHKKPTG MIRIHQMNSE LSVLANFSQP EIVPISNITE | 120 |
| NVYINLTCSS IHGYPEPKKM SVLLRTKNST IEYDGIMQKS QDNVTELYDV SISLSVSFPD | 180 |
| VTSNMTIFCI LETDKTRLLS SPFSIELEDP QPPPDHIPWI TAVLPTVIIC VMVFCLILWK | 240 |
| WKKKKRPRNS YKCGTNTMER EESEQTKKRE KIHIPERSDE AQRVFKSSKT SSCDKSDTCF. | 300 |

It will be appreciated that SEQ ID NOs: 11 and 13 each contain a signal peptide.

B 7-H5

Murine B7-H5 polypeptides can have at least 80%, 85%, 90%, 95%, 99% or 100% sequence identity to:

| (SEQ ID NO: 15) | |
|---|---|
| MGVPAVPEAS SPRWGTLLLA IFLAASRGLV AAFKVTTPYS LYVCPEGQNA TLTCRILGPV | 60 |
| SKGHDVTIYK TWYLSSRGEV QMCKEHRPIR NFTLQHLQHH GSHLKANASH DQPQKHGLEL | 120 |
| ASDHHGNFSI TLRNVTPRDS GLYCCLVIEL KNHHPEQRFY GSMELQVQAG KGSGSTCMAS | 180 |
| NEQDSDSITA AALATGACIV GILCLPLILL LVYKQRQVAS HRRAQELVRM DSSNTQGIEN | 240 |
| PGFETTPPFQ GMPEAKTRPP LSYVAQRQPS ESGRYLLSDP STPLSPPGPG DVFFPSLDPV | 300 |
| PDSPNSEAI | 309 |
| or | |
| (SEQ ID NO: 16) | |
| FKVTTPYSLY VCPEGQNATL TCRILGPVSK GHDVTIYKTW YLSSRGEVQM CKEHRPIRNF | 60 |
| TLQHLQHHGS HLKANASHDQ PQKHGLELAS DHHGNESITL RNVTPRDSGL YCCLVIELKN | 120 |
| HHPEQRFYGS MELQVQACKG SGSTCMASNE QDSDSITARA LATGACIVGI LCLPLILLLV | 180 |
| YKQRQVASHR RAQELVRMDS SNTOGIENPG FETTPPFQGM PEAKTRPPLS YVAQRQPSES | 240 |
| GRYLLSDPST PLSPPGPGDV FFPSLDPVPD SPNSEAI. | 277 |

Human B7-H5 can have at least 80%, 85%, 90%, 95%, 99% or 100% sequence identity to:

| (SEQ ID NO: 17) | |
|---|---|
| MGVPTALEAG SWRWGSLLFA LFLAASLGPV AAFKVATPYS LYVCPEGQNV TLTCRLLGPV | 60 |
| DKGHDVTFYK TWYRSSRGEV QTCSERRPIR NLTFQDLHLH HGGHQAANTS HDLAQRHGLE | 120 |
| SASDHHGNFS ITMRNLTLLD SGLYCCLVVE IRHHHSEHRV HGAMELQVQT GKDAPSNCVV | 180 |
| YPSSSQDSEN ITAAALATGA CIVGILCLPL ILLLVYKQRQ AASNRRAQEL VRMDSNIQGI | 240 |
| ENPGFEASPP AQGIPEAKVR HPLSYVAQRQ PSESGRHLLS EPSTPLSPPG PGDVFFPSLD | 300 |
| PVPDSPNFEV I | 311 |
| or | |
| (SEQ ID NO: 18) | |
| FKVATPYSLY VCPEGQNVTL TCRLLGPVDR GHDVTFYKTW YRSSRGEVQT CSERRPIRNL | 60 |
| TFQDLHLHHG GHQAANTSHD LAQRHGLESA SDHHGNFSIT MRNLTLLDSG LYCCLVVEIR | 120 |
| HHHSEHRVHG AMELQVQTGK DAPSNCVVYP SSSQDSENIT AAALATGACT VGILCLPLIL | 180 |
| LLVYKQRQAA SNRRAQELVR MDSUIQGIEN PGFEASPPAQ GIPEAKVRHP LSYVAQRQPS | 240 |
| ESGRHLLSEP STPLSPPGPG DVFFPSLDPV PDSPNFEVI. | 279 |

It will be appreciated that SEQ ID NOs: 15 and 17 each contain a signal peptide.

a. Murine B7 Costimulatory Extracellular Domains

In one embodiment, the disclosed fusion proteins include the extracellular domain of the murine B7-DC, B7-1, B7-2 or B7-H5, proteins shown in SEQ ID NOs:1, 2, 7, 8, 11, 12, 15 or 16, as shown below.

B7-DC

The costimulatory polypeptide domain of the fusion protein can be encoded by a nucleotide sequence having at least 80%, 85%, 90%, 95%, 99%, or 100% sequence identity to:

| (SEQ ID NO: 19) | |
|---|---|
| atgctgctcc tgctgccgat actgaacctg agcttacaac ttcatcctgt agcagcttta | 60 |
| ttcaccgtga cagcccctaa agaagtgtac accgtagacg tcggcagcag tgtgagcctg | 120 |
| gagtgcgatt ttgaccgcag agaatgcact gaactggaag ggataagagc cagtttgcag | 180 |
| aaggtagaaa atgatacgtc tctgcaaagt gaaagagcca ccctgctgga ggagcagctg | 240 |
| cccctgggaa aggctttgtt ccacatccct agtgtccaag tgagagattc cgggcagtac | 300 |
| cgttgcctgg tcatctgcgg ggccgcctgg gactacaagt acctgacggt gaaagtcaaa | 360 |
| gcttcttaca tgaggataga cactaggatc ctggaggttc caggtacagg ggaggtgcag | 420 |
| cttacctgcc aggctagagg ttatccccta gcagaagtgt cctggcaaaa tgtcagtgtt | 480 |
| cctgccaaca ccagccacat caggaccccc gaaggcctct accaggtcac cagtgttctg | 540 |
| cgcctcaagc ctcagcctag cagaaacttc agctgcatgt tctggaatgc tcacatgaag | 600 |
| gagctgactt cagccatcat tgaccctctg agtcggatgg aacccaaagt ccccagaacg | 660 |
| tgg. | 663 |

In another embodiment, the costimulatory polypeptide domain of the fusion protein can have at least 80%, 85%, 90%, 95%, 99%, or 100% sequence identity to:

| (SEQ ID NO: 20) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT W. | 221 |

It will be appreciated that the signal sequence will be removed in the mature protein. Additionally, it will be appreciated that signal peptides from other organisms can be used to enhance the secretion of the fusion protein from a host during manufacture. SEQ ID NO:21 provides the murine amino acid sequence of SEQ ID NO:20 without the signal sequence:

| (SEQ ID NO: 21) | |
|---|---|
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMEWNABM | 180 |
| KELTSAIIDP LSRMEPKVPR TW. | 202 |

In another embodiment, the costimulatory polypeptide domain of the fusion protein includes the IgV domain of murine B7-DC. The costimulatory polypeptide domain can be encoded by a nucleotide sequence having at least 80%, 85%, 90%, 95%, 99%, or 100% sequence identity to:

| (SEQ ID NO: 22) | |
|---|---|
| ttcaccgtga cagcccctaa agaagtgtac accgtagacg tcggcagcag tgtgagcctg | 60 |
| gagtgcgatt ttgaccgcag agaatgcact gaactggaag ggataagagc cagtttgcag | 120 |
| aaggtagaaa atgatacgtc tctgcaaagt gaaagagcca ccctgctgga ggagcagctg | 180 |
| cccctgggaa aggctttgtt ccacatccct agtgtccaag tgagagattc cgggcagtac | 240 |
| cgttgcctgg tcatctgcgg ggccgcctgg gactacaagt acctgacggt gaaa. | 294 |

The costimulatory polypeptide domain of the fusion protein can have at least 80%, 85%, 90%, 95%, 99%, or 100% sequence identity to:

| (SEQ ID NO: 23), also referred to as B7-DCV | |
|---|---|
| FTVTAPKEVY TVDVGSSVSL ECDFDRRECT ELEGIRASLQ KVENDTSLQS ERATLLEEQL | 60 |
| PLGKALFHIP SVQVRDSGQY RCLVICGAAW DYKYLTVK. | 98 |

B7-1

The costimulatory polypeptide domain of the fusion protein can be encoded by a nucleotide sequence having at least 80%, 85%, 90%, 95%, 99%, or 100% sequence identity to:

| (SEQ ID NO: 24) | |
|---|---|
| atggcttgca attgtcagtt gatgcaggat acaccactcc tcaagtttcc atgtccaagg | 60 |
| ctcattcttc tctttgtgct gctgattcgt ctttcacaag tgtcttcaga tgttgatgaa | 120 |
| caactgtcca agtcagtgaa agataaggta ttgctgcctt gccgttacaa ctctcctcat | 180 |
| gaagatgagt ctgaagaccg aatctactgg caaaaacatg acaaagtggt gctgtctgtc | 240 |
| attgctggga aactaaaagt gtggcccgag tataagaacc ggactttata tgacaacact | 300 |
| acctactctc ttatcatcct gggcctggtc ctttcagacc ggggcacata cagctgtgtc | 360 |
| gttcaaaaga aggaaagagg aacgtatgaa gttaaacact tggctttagt aaagttgtcc | 420 |
| atcaaagctg acttctctac ccccaacata actgagtctg gaaacccatc tgcagacact | 480 |
| aaaaggatta cctgctttgc ttccgggggt ttcccaaagc ctcgcttctc ttggttggaa | 540 |
| aatggaagag aattacctgg catcaatacg acaatttccc aggatcctga atctgaattg | 600 |
| tacaccatta gtagccaact agatttcaat acgactcgca accacaccat taagtgtctc | 660 |
| attaaatatg gagatgctca cgtgtcagag gacttcacct gggaaaaacc cccagaagac | 720 |
| cctcctgata gcaagaac. | 738 |

In another embodiment, the costimulatory polypeptide domain of the fusion protein can have at least 80%, 85%, 90%, 95%, 99%, or 100% sequence identity to:

| (SEQ ID NO: 25) | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLIILGLV LSDRGTYSCV | 120 |
| VQKKERGTYE VKHLALVKLS IKADFSTPNI TESGNPSADT KRITCFASGG FPKPRFSWLE | 180 |
| NGRELPGINT TISQDPESEL YTISSQLDFN TTRNHTIKCL IKYGDAHVSE DFTWEKPPED | 240 |
| PPDSKN. | 246 |

It will be appreciated that the signal sequence will be removed in the mature protein. Additionally, it will be appreciated that signal peptides from other organisms can be used to enhance the secretion of the fusion protein from a host during manufacture. SEQ ID NO:26 provides the murine amino acid sequence of SEQ ID NO:25 without the signal sequence:

| (SEQ ID NO: 26) | |
|---|---|
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHLALV KLSIKADFST PNITESGNPS | 120 |
| ADTKRITCFA SGGFPKPRFS WLENGRELPG INTTISQDPE SELYTISSQL DENTTRNHTI | 180 |
| KCLIKYGDAH VSEDFTWEKP PEDPPDSKN. | 209 |

In another embodiment, the costimulatory polypeptide domain of the fusion protein includes the IgV domain of murine B7-1. The costimulatory polypeptide domain can be encoded by a nucleotide sequence having at least 80%, 85%, 90%, 95%, 99%, or 100% sequence identity to:

| (SEQ ID NO: 27) | |
|---|---|
| gttgatgaac aactgtccaa gtcagtgaaa gataaggtat tgctgccttg ccgttacaac | 60 |
| tctcctcatg aagatgagtc tgaagaccga atctactggc aaaaacatga caaagtggtg | 120 |
| ctgtctgtca ttgctgggaa actaaaagtg tggcccgagt ataagaaccg gactttatat | 180 |
| gacaacacta cctactctct tatcatcctg ggcctggtcc tttcagaccg gggcacatac | 240 |
| agctgtgtcg ttcaaaagaa ggaaagagga acgtatgaag ttaaacactt g. | 291 |

The costimulatory polypeptide domain of the fusion protein can have at least 80%, 85%, 90%, 95%, 99%, or 100% sequence identity to:

| (SEQ ID NO: 28), also referred to as B7-1V | |
|---|---|
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHL. | 97 |

B7-2

The costimulatory polypeptide domain of the fusion protein can be encoded by a nucleotide sequence having at least 80%, 85%, 90%, 95%, 99%, or 100% sequence identity to:

| (SEQ ID NO: 29) | |
|---|---|
| atggacccca gatgcaccat gggcttggca atccttatct ttgtgacagt cttgctgatc | 60 |
| tcagatgctg tttccgtgga gacgcaagct tatttcaatg ggactgcata tctgccgtgc | 120 |
| ccatttacaa aggctcaaaa cataagcctg agtgagctgg tagtattttg gcaggaccag | 180 |
| caaaagttgg ttctgtacga gcactatttg ggcacagaga aacttgatag tgtgaatgcc | 240 |
| aagtacctgg gccgcacgag ctttgacagg aacaactgga ctctacgact tcacaatgtt | 300 |
| cagatcaagg acatgggctc gtatgattgt tttatacaaa aaaagccacc cacaggatca | 360 |
| attatcctcc aacagacatt aacagaactg tcagtgatcg ccaacttcag tgaacctgaa | 420 |
| ataaaactgg ctcagaatgt aacaggaaat tctggcataa atttgacctg cacgtctaag | 480 |
| caaggtcacc cgaaacctaa gaagatgtat tttctgataa ctaattcaac taatgagtat | 540 |
| ggtgataaca tgcagatatc acaagataat gtcacagaac tgttcagtat ctccaacagc | 600 |
| ctctctcttt cattcccgga tggtgtgtgg catatgaccg ttgtgtgtgt tctggaaacg | 660 |
| gagtcaatga agatttcctc caaacctctc aatttcactc aagagtttcc atctcctcaa | 720 |
| acgtattgga ag. | 732 |

In another embodiment, the costimulatory polypeptide domain of the fusion protein can have at least 80%, 85%, 90%, 95%, 99%, or 100% sequence identity to:

| (SEQ ID NO: 30) | |
|---|---|
| MDPRCTMGLA ILIFVTVLLI SDAVSVETQA YFNGTAYLPC PFTKAQNISL SELVVFWQDQ | 60 |
| QKLVLYEHYL GTEKLDSVNA KYLGRTSFDR NNWTLRLHNV QIKDMGSYDC FIQKKPPTGS | 120 |
| IILQQTLTEL SVIANFSEPE IKLAQNVTGN SGINLTCTSK QGHPKPKKMY FLITNSTNEY | 180 |
| GDNMQISQDN VTELFSISNS LSLSFPDGVW HMTVVCVLET ESMKISSKPL NFTQEEPSPQ | 240 |
| TYWK. | 244 |

It will be appreciated that the signal sequence will be removed in the mature protein. Additionally, it will be appreciated that signal peptides from other organisms can be used to enhance the secretion of the fusion protein from a host during manufacture. SEQ ID NO:31 provides the murine amino acid sequence of SEQ ID NO:30 without the signal sequence:

| (SEQ ID NO: 31) | |
|---|---|
| VSVETQAYFN GTAYLPCPFT KAQNISLSEL VVFWQDQQKL VLYEHYLGTE KLDSVNAKYL | 60 |
| GRTSFDRNNW TLRLHNVQIK DMGSYDCFIQ KKPPTGSIIL QQTLTELSVI ANFSEPEIKL | 120 |
| AQNVTGNSGI NLTCTSKQGH PKPKKMYFLI TNSTNEYGDN MQISQDNVTE LFSISNSLSL | 180 |
| SFPDGVWHMT VVCVLETESM KISSKPLNFT QEFPSPQTYW K. | 221 |

In another embodiment, the costimulatory polypeptide domain of the fusion protein includes the IgV domain of murine B7-2. The costimulatory polypeptide domain can be encoded by a nucleotide sequence having at least 80%, 85%, 90%, 95%, 99%, or 100% sequence identity to:

| (SEQ ID NO: 32) | |
|---|---|
| aatgggactg catatctgcc gtgcccattt acaaaggctc aaaacataag cctgagtgag | 60 |
| ctggtagtat tttggcagga ccagcaaaag ttggttctgt acgagcacta tttgggcaca | 120 |
| gagaaacttg atagtgtgaa tgccaagtac ctgggccgca cgagctttga caggaacaac | 180 |
| tggactctac gacttcacaa tgttcagatc aaggacatgg gctcgtatga ttgttttata | 240 |
| caaaaaaagc cacccacagg atcaattatc ctccaacaga cattaaca. | 288 |

The costimulatory polypeptide domain of the fusion protein can have at least 80%, 85%, 90%, 95%, 99%, or 100% sequence identity to:

| (SEQ ID NO: 33), also referred to as 137-2V | |
|---|---|
| NGTAYLPCPF TKAQNISLSE LVVFWQDQQK LVLYEHYLGT EKLDSVNAKY LGRTSFDRNN | 60 |
| WTLRLHNVQI KDMGSYDCFI QKKPPTGSII LQQTLT. | 96 |

B7-H5

The costimulatory polypeptide domain of the fusion protein can be encoded by a nucleotide sequence having at least 80%, 85%, 90%, 95%, 99%, or 100% sequence identity to:

| (SEQ ID NO: 34) | |
|---|---|
| atgggtgtcc ccgcggtccc agaggccagc agcccgcgct ggggaaccct gctccttgct | 60 |
| attttcctgg ctgcatccag aggtctggta gcagccttca aggtcaccac tccatattct | 120 |
| ctctatgtgt gtcccgaggg acagaatgcc accctcacct gcaggattct gggccccgtg | 180 |
| tccaaagggc acgatgtgac catctacaag acgtggtacc tcagctcacg aggcgaggtc | 240 |
| cagatgtgca aagaacaccg gcccatacgc aacttcacat tgcagcacct tcagcaccac | 300 |
| ggaagccacc tgaaagccaa cgccagccat gaccagcccc agaagcatgg gctagagcta | 360 |
| gcttctgacc accacggtaa cttctctatc accctgcgca atgtgacccc aagggacagc | 420 |
| ggcctctact gctgtctagt gatagaatta aaaaaccacc acccagaaca acggttctac | 480 |
| gggtccatgg agctacaggt acaggcaggc aaaggctcgg ggtccacatg catggcgtct | 540 |
| aatgagcagg acagtgacag catcacggct. | 570 |

In another embodiment, the costimulatory polypeptide domain of the fusion protein can have at least 80%, 85%, 90%, 95%, 99%, or 100% sequence identity to:

| (SEQ ID NO: 35) | |
|---|---|
| MGVPAVPEAS SPRWGTLLLA IFLAASRGLV AAFKVTTPYS LYVCPEGQNA TLTCRILGPV | 60 |
| SKGHDVTIYK TWYLSSRGEV QMCKEHRPIR NFTLQHLQHH GSHLKANASH DQPQKHGLEL | 120 |
| ASDHHGNFSI TLRNVTPRDS GLYCCLVIEL KNHHPEQRFY GSMELQVQAG KGSGSTCMAS | 180 |
| NEQDSDSITA. | 190 |

It will be appreciated that the signal sequence will be removed in the mature protein. Additionally, it will be appreciated that signal peptides from other organisms can be used to enhance the secretion of the fusion protein from a host during manufacture. SEQ ID NO:36 provides the murine amino acid sequence of SEQ ID NO:35 without the signal sequence:

| (SEQ ID NO: 36) | |
|---|---|
| FKVTTPYSLY VCPEGQNATL TCRILGPVSK GHDVTIYKTW YLSSRGEVQM CKEHRPIRNF | 60 |
| TLQHLQHHGS HLKANASHDQ PQKHGLELAS DHHGNFSITL RNVTPRDSGL YCCLVIELKN | 120 |
| HHPEQRFYGS MELQVQAGKG SGSTCMASNE QDSDSITA. | 158 |

In another embodiment, the costimulatory polypeptide domain of the fusion protein includes the IgV domain of murine B7-H5. The costimulatory polypeptide domain can be encoded by a nucleotide sequence having at least 80%, 85%, 90%, 95%, 99%, or 100% sequence identity to:

| (SEQ ID NO: 37) | |
|---|---|
| ttcaaggtca ccactccata ttctctctat gtgtgtcccg agggacagaa tgccaccctc | 60 |
| acctgcagga ttctgggccc cgtgtccaaa gggcacgatg tgaccatcta caagacgtgg | 120 |
| tacctcagct cacgaggcga ggtccagatg tgcaaagaac accggcccat acgcaacttc | 180 |
| acattgcagc accttcagca ccacggaagc cacctgaaag ccaacgccag ccatgaccag | 240 |
| ccccagaagc atgggctaga gctagcttct gaccaccacg gtaacttctc tatcaccctg | 300 |
| cgcaatgtga ccccaaggga cagcggcctc tactgctgtc tagtgataga attaaaaaac | 360 |
| caccacccag aacaacggtt ctacggg. | 387 |

The T cell receptor binding domain of the fusion protein can have at least 80%, 85%, 90%, 95%, 99%, or 100% sequence identity to:

| (SEQ ID NO: 36), also referred to as B7-HSV. | |
|---|---|
| FKVTTPYSLY VCPEGQNATL TCRILGPVSK GHDVTIYKTW YLSSRGEVQM CKEHRPIRNF | 60 |
| TLQHLQHHGS HLKANASHDQ PQKHGLELAS DHHGNESTTL RNVTPRDSGL YCCLVIELKN | 120 |
| HHPEQRFYG | 129 |

b. Human B7 Costimulatory Extracellular Domains

In one embodiment, the disclosed fusion proteins include the extracellular domain of the human B7-DC, B7-1, B7-2 or B7-H5, proteins shown in SEQ ID NOs:3, 4, 9, 10, 13, 14, 15 or 16, as shown below.

B7-DC

The costimulatory polypeptide domain of the fusion protein can be encoded by a nucleotide sequence having at least 80%, 85%, 90%, 95%, 99%, or 100% sequence identity to:

| (SEQ ID NO: 39) | |
|---|---|
| atgatctttc ttctcttgat gctgtctttg gaattgcaac ttcaccaaat cgcggccctc | 60 |
| tttactgtga ccgtgccaaa agaactgtat atcattgagc acgggtccaa tgtgaccctc | 120 |
| gaatgtaact ttgacaccgg cagccacgtt aacctggggg ccatcactgc cagcttgcaa | 180 |
| aaagttgaaa acgacacttc acctcaccgg gagagggcaa ccctcttgga ggagcaactg | 240 |
| ccattgggga aggcctcctt tcatatccct caggtgcagg ttcgggatga gggacagtac | 300 |
| cagtgcatta ttatctacgg cgtggcttgg gattacaagt atctgaccct gaaggtgaaa | 360 |
| gcgtcctatc ggaaaattaa cactcacatt cttaaggtgc cagagacgga cgaggtggaa | 420 |
| ctgacatgcc aagccaccgg ctacccgttg gcagaggtca gctggcccaa cgtgagcgta | 480 |
| cotgotaaca cttctcattc taggacaccc gagggcctct accaggttac atccgtgctc | 540 |
| cgcctcaaac egccuccagg ccggaatttt agttgcgtgt tttggaatac ccacgtgcga | 600 |
| gagctgactc ttgcatctat tgatctgcag tcccagatgg agccacggac tcatccaact | 660 |
| tgg | 663. |

In another embodiment, the costimulatory polypeptide domain of the fusion protein can have at least 80%, 85%, 90%, 95%, 99%, or 100% sequence identity to:

| (SEQ ID NO: 40) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W | 221. |

It will be appreciated that the signal sequence will be removed in the mature protein. Additionally, it will be appreciated that signal peptides from other organisms can be used to enhance the secretion of the fusion protein from a host during manufacture. SEQ ID NO:41 provides the human amino acid sequence of SEQ ID NO:40 without the signal sequence:

| (SEQ ID NO: 41) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW | 202. |

In another embodiment, the costimulatory polypeptide domain of the fusion protein includes the IgV domain of human B7-DC. The costimulatory polypeptide domain can be encoded by a nucleotide sequence having at least 80%, 85%, 90%, 95%, 99%, or 100% sequence identity to:

| (SEQ ID NO: 42) | |
|---|---|
| tttactgtga ccgtgccaaa agaactgtat atcattgagc acgggtccaa tgtgaccctc | 60 |
| gaatgtaact ttgacaccgg cagccacgtt aacctggggg ccatcactgc cagcttgcaa | 120 |
| aaagttgaaa acgacacttc acctcaccgg gagagggcaa ccctcttgga ggagcaactg | 180 |
| ccattgggga aggcctcctt tcatatccct caggtgcagg ttcgggatga gggacagtac | 240 |
| cagtgcatta ttatctacgg cgtggcttgg gattacaagt atctgaccct gaag | 294. |

The costimulatory polypeptide domain of the fusion protein can have at least 80%, 85%, 90%, 95%, 99%, or 100% sequence identity to:

| (SEQ ID NO: 43), also referred to as B7-DC. | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW DYKYLTLK | 98 |

B7-1

The costimulatory polypeptide domain of the fusion protein can be encoded by a nucleotide sequence having at least 80%, 85%, 90%, 95%, 99%, or 100% sequence identity to:

| (SEQ ID NO: 44) | |
|---|---|
| atgggccaca cacggaggca gggaacatca ccatccaagt gtccatacct caatttcttt | 60 |
| cagctcttgg tgctggctgg tctttotcac ttctgttcag gtgttatcca cgtgaccaag | 120 |
| gaagtgaaag aagtggcaac gctgtcctgt ggtcacaatg tttctgttga agagctggca | 180 |
| caaactcgca tctactggca aaaggagaag aaaatggtgc tgactatgat gtctggggac | 240 |
| atgaatatat ggcccgagta caagaaccgg accatctttg atatcactaa taacctctcc | 300 |
| attgtgatcc tggctctgcg cccatctgac gagggcacat acgagtgtgt tgttctgaag | 360 |
| tatgaaaaag acgctttcaa gcgggaacac ctggctgaag tgacgttatc agtcaaagct | 420 |
| gacttcccta cacctagtat atctgacttt gaaattccaa cttctaatat tagaaggata | 480 |
| atttgctcaa cctctggagg ttttccagag cctcacctct cctggttgga aaatggagaa | 540 |
| gaattaaatg ccatcaacac aacagtttcc caagatcctg aaactgagct ctatgctgtt | 600 |
| agcagcaaac tggatttcaa tatgacaacc aaccacagct tcatgtgtct catcaagtat | 660 |
| ggacatttaa gagtgaatca gaccttcaac tggaatacaa ccaagcaaga gcattttcct | 720 |
| gataacctgc tc | 732. |

In another embodiment, the costimulatory polypeptide domain of the fusion protein can have at least 80%, 85%, 90%, 95%, 99%, or 100% sequence identity to:

| (SEQ ID NO: 45) | |
|---|---|
| MGHTRRQGTS PSKCPYLHFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPS1SDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNL | 243. |

It will be appreciated that the signal sequence will be removed in the mature protein. Additionally, it will be appreciated that signal peptides from other organisms can be used to enhance the secretion of the fusion protein from a host during manufacture. SEQ ID NO:46 provides the murine amino acid sequence of SEQ ID NO:45 without the signal sequence:

| (SEQ ID NO: 46) | |
|---|---|
| VIHVTKEVKE VATLSCGHNV SVEELAQTRI YWQKEKKMVL TMMSGDMNIW PEYKNRTIFD | 60 |
| ITHHLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNL | 209. |

In another embodiment, the costimulatory polypeptide domain of the fusion protein includes the IgV domain of human B7-1. The costimulatory polypeptide domain can be encoded by a nucleotide sequence having at least 80%, 85%, 90%, 95%, 99%, or 100% sequence identity to:

| (SEQ ID NO: 47) | |
|---|---|
| gttatccacg tgaccaagga agtgaaagaa gtggcaacgc tgtcctgtgg tcacaatgtt | 60 |
| tctgttgaag agctggcaca aactcgcatc tactggcaaa aggagaagaa aatggtgctg | 120 |
| actatgatgt ctggggacat gaatatatgg cccgagtaca agaaccggac catctttgat | 180 |
| atcactaata acctctccattgtgatcctg gctctgcgcc catctgacga gggcacatac | 240 |
| gagtgtgttg ttctgaagta tgaaaaagac gctttcaagc gggaacacct ggctgaagtg | 300 |
| acg | 303. |

The costimulatory polypeptide domain of the fusion protein can have at least 80%, 85%, 90%, 95%, 99%, or 100% sequence identity to:

| (SEQ ID NO: 48), also referred to as B7-1. | |
|---|---|
| VIHVTKEVKE VATLSCGHNV SVEELAQTRI YWQKEKKMVL TMMSGDMNIW PEYKNRTIFD | 60 |
| ITNNLSIVIL ALRFSDEGTY ECVVLKYEKD AFKREHLAEV T | 101 |

B7-2

The costimulatory polypeptide domain of the fusion protein can be encoded by a nucleotide sequence having at least 80%, 85%, 90%, 95%, 99%, or 100% sequence identity to:

| (SEQ ID NO: 49) | |
|---|---|
| atgggactga gtaacattct ctttgtgatg gccttcctgc tctctggtgc tgctcctctg | 60 |
| aagattcaag cttatttcaa tgagactgca gacctgccat gccaatttgc aaactctcaa | 120 |
| aaccaaagcc tgagtgagct agtagtattt tggcaggacc aggaaaactt ggttctgaat | 180 |
| gaggtatact taggcaaaga gaaatttgac agtgttcatt ccaagtatat gggccgcaca | 240 |
| agttttgatt cggacagttg gaccctgaga cttcacaatc ttcagatcaa ggacaagggc | 300 |
| ttgtatcaat gtatcatcca tcacaaaaag cccacaggaa tgattcgcat ocaccagatg | 360 |
| aattctgaac tgtcagtgct tgctaacttc agtcaacctg aaatagtacc aatttctaat | 420 |
| ataacagaaa atgtgtacat aaatttgacc tgctcatcta tacacggtta cccagaacct | 480 |
| aagaagatga gtgttttgct aagaaccaag aattcaacta tcgagtatga tggtgttatg | 540 |
| cagaaatctc aagataatgt cacagaactg tacgacgttt ccatcagctt gtctgtttca | 600 |
| ttccctgatg ttacgagcaa tatgaccatc ttctgtattc tggaaactga caagacgcgg | 660 |
| cttttatctt cacctttctc tatagagctt gaggaccctc agcctccccc agaccacatt | 720 |
| ccttggatta cagctgtact t | 741. |

In another embodiment, the costimulatory polypeptide domain of the fusion protein can have at least 80%, 85%, 90%, 95%, 99%, or 100% sequence identity to:

| (SEQ ID NO: 50) | |
|---|---|
| MGLSNILFVM AFLLSGAAPL KIQAYFNETA DLPCQFANSQ NQSLSELVVF WQDQENLVLN | 60 |
| EVYLGKEKFD SVHSKYMGRT SFDSDSWTLR LHNLQIKDKG LYQCIIHHKK PTGMIRIHQM | 120 |
| NSELSVLANF SQPEIVPISN ITENVYINLT CSSIHGYPEP KKMSVLLRTK NSTIEYDGVM | 180 |
| QKSQDNVTEL YDVSISISVS FPDVTSNMTI FCILETDKTR LLSSPFSIEL EDPQPPPDHI | 240 |
| PWITAVL | 247. |

It will be appreciated that the signal sequence will be removed in the mature protein. Additionally, it will be appreciated that signal peptides from other organisms can be used to enhance the secretion of the fusion protein from a host during manufacture. SEQ ID NO:51 provides the murine amino acid sequence of SEQ ID NO:50 without the signal sequence:

| (SEQ ID NO: 51) | |
|---|---|
| AYFNETADLP CQFANSQNQS LSELVVFWQD QENLVLNEVY LGKEKFDSVH SKYMGRTSFD | 60 |
| SDSWTLRLHN LQIKDKGLYQ CIIHHKKPTG MIRIHQMNSE LSVLANFSQP EIVPISNITE | 120 |
| NVYINLTCSS IHGYPEPKKM SVLLRTKNST IEYDGVMQKS QDNVTELYDV SISLSVSFPD | 180 |
| VTSNMTIFCI LETDKTRLLS SPFSIELEDP QPPPDHIPWI TAVL | 224. |

In another embodiment, the costimulatory polypeptide domain of the fusion protein includes the IgV domain of human B7-2. The costimulatory polypeptide domain can be encoded by a nucleotide sequence having at least 80%, 85%, 90%, 95%, 99%, or 100% sequence identity to:

| (SEQ ID NO: 52) | |
|---|---|
| ccatgccaat ttgcaaactc tcaaaaccaa agcctgagtg agctagtagt attttggcag | 60 |
| gaccaggaaa acttggttct gaatgaggta tacttaggca aagagaaatt tgacagtgtt | 120 |
| cattccaagt atatgggccg cacaagtttt gattcggaca gttggaccct gagacttcac | 180 |
| aatcttcaga tcaaggacaa gggcttgtat caatgtatca tccatcacaa aaagcccaca | 240 |
| ggaatgattc gcatccacca gatgaattct gaactgtcag tgcttgctaa cttc | 294. |

The costimulatory polypeptide domain of the fusion protein can have at least 80%, 85%, 90%, 95%, 99%, or 100% sequence identity to:

| (SEQ ID NO: 53), also referred to as B7-2V | |
|---|---|
| PCQFANSQNQ SLSELVVFWQ DQENLVLNEV YLGKEKFDSV HSKYMGRTSF DSDSWTLRLH | 60 |
| NLQIKDKGLY QCIIHHKKPT GMIRIHQMNS ELSVLANF. | 98 |

B7-H5

The costimulatory polypeptide domain of the fusion protein can be encoded by a nucleotide sequence having at least 80%, 85%, 90%, 95%, 99%, or 100% sequence identity to:

| (SEQ ID NO: 54) | |
|---|---|
| atgggcgtcc ccacggccct ggaggccggc agctggcgct ggggatccct gctcttcgct | 60 |
| ctcttcctgg ctgcgtccct aggtccggtg gcagccttca aggtcgccac gccgtattcc | 120 |
| ctgtatgtct gtcccgaggg gcagaacgtc accctcacct gcaggctctt gggccctgtg | 180 |
| gacaaagggc acgatgtgac cttctacaag acgtggtacc gcagctcgag gggcgaggtg | 240 |
| cagacctgct cagagcgccg gcccatccgc aacctcacgt tccaggacct tcacctgcac | 300 |
| catggaggcc accaggctgc caacaccagc cacgacctgg ctcagcgcca cgggctggag | 360 |
| tcggcctccg accaccatgg caacttctcc atcaccatgc gcaacctgac cctgctggat | 420 |
| agcggcctct actgctgcct ggtggtggag atcaggcacc accactcgga gcacagggtc | 480 |
| catggtgcca tggagctgca ggtgcagaca ggcaaagatg caccatccaa ctgtgtggtg | 540 |
| tacccatcct cctcccagga tagtgaaaac atcacggct. | 579 |

In another embodiment, the costimulatory polypeptide domain of the fusion protein can have at least 80%, 85%, 90%, 95%, 99%, or 100% sequence identity to:

| (SEQ ID NO: 55) | |
|---|---|
| MGVPTALEAG SWRWGSLLFA LFLAASLGPV AAFKVATPYS LYVCPEGQNV TLTCRLLGPV | 60 |
| DKGHDVTFYK TWYRSSRGEV QTCSERRPIR NLTFQDLHLH HGGHQAANTS HDLAQRHGLE | 120 |
| SASDHHGNFS ITMRNLTLLD SGLYCCLVVE IRHHHSEHRV HGAMELQVQT GKDAPSNCVV | 180 |
| YPSSSQDSEN ITA. | 193 |

It will be appreciated that the signal sequence will be removed in the mature protein. Additionally, it will be appreciated that signal peptides from other organisms can be used to enhance the secretion of the fusion protein from a host during manufacture. SEQ ID NO:56 provides the murine amino acid sequence of SEQ ID NO:55 without the signal sequence:

| (SEQ ID NO: 56) | |
|---|---|
| FKVATPYSLY VCPEGQNVTL TCRLLGPVDK GHDVTFYKTW YRSSRGEVQT CSERRPIRNL | 60 |
| TFOLHLLHHG GHQAANTSHD LAQRHGLESA SDHHGNFSIT MRNLTLLDSG LYCCLVVEIR | 120 |
| HHHSEHRVHG AMELQVQTGK DAPSNCVVYP SSSQDSENIT A. | 161 |

In another embodiment, the costimulatory polypeptide domain of the fusion protein includes the IgV domain of human B7-H5. The costimulatory polypeptide domain can be encoded by a nucleotide sequence having at least 80%, 85%, 90%, 95%, 99%, or 100% sequence identity to:

| (SEQ ID NO: 57) | |
|---|---|
| ttcaaggtcg ccacgccgta ttccctgtat gtctgtcccg aggggcagaa cgtcaccctc | 60 |
| acctgcaggc tcttgggccc tgtggacaaa gggcacgatg tgaccttcta caagacgtgg | 120 |
| taccgcagct cgaggggcga ggtgcagacc tgctcagagc gccggcccat ccgcaacctc | 180 |
| acgttccagg accttcacct gcaccatgga ggccaccagg ctgccaacac cagccacgac | 240 |
| ctggctcagc gccacgggct ggagtcggcc tccgaccacc atggcaactt ctccatcacc | 300 |
| atgcgcaacc tgaccctgct ggatagcggc ctctactgct gcctggtggt ggagatcagg | 360 |
| caccaccact cggagcacag ggtccatggt. | 390 |

The costimulatory polypeptide domain of the fusion protein can have at least 80%, 85%, 90%, 95%, 99%, or 100% sequence identity to:

| (SEQ ID NO: 58), also referred to as B7-HSV | |
|---|---|
| FKVATPYSLY VCPEGQNVTL TCRLLGPVDK GHDVTFYKTW YRSSRGEVQT CSERRPIRNL | 60 |
| TFQDLHLHHG GHQAANTSHD LAQRHGLESA SDHHGNFSIT MRNLTLLDSG LYCCLVVEIR | 120 |
| HHHSEHRVHG. | 130 |

c. Non-Human Primate B7-DC Costimulatory Extracellular Domains

In one embodiment, the disclosed fusion proteins include the extracellular domain of the non-human primate (Cynomolgus) proteins shown in SEQ ID NOs:5 or 6, as shown below.

B7-DC

The costimulatory polypeptide domain of the fusion protein can be encoded by a nucleotide sequence having at least 80%, 85%, 90%, 95%, 99%, or 100% sequence identity to:

| (SEQ ID NO: 59) | |
|---|---|
| atgatcttcc tcctgctaat gttgagcctg gaattgcagc ttcaccagat agcagcttta | 60 |
| ttcacagtga cagtccctaa ggaactgtac ataatagagc atggcagoaa tgtgaccctg | 120 |
| gaatgcaact ttgacactgg aagtcatgtg aaccttggag caataacagc cagtttgcaa | 180 |
| aaggtggaaa atgatacatc cccacaccgt gaaagagcca ctttgctgga ggagcagctg | 240 |
| cccctaggga aggcctcgtt ccacatacct caagtccaag tgagggacga aggacagtac | 300 |
| caatgcataa tcatctatgg ggtcgcctgg gactacaagt acctgactct gaaagtcaaa | 360 |
| gcttcctaca ggaaaataaa cactcacatc ctaaaggttc cagaaacaga tgaggtagag | 420 |
| ctcacctgcc aggctacagg ttatcctctg gcagaagtat cctggccaaa cgtcagcgtt | 480 |
| cctgccaaca ccagccactc caggacccct gaaggcctct accaggtcac cagtgttctg | 540 |
| cgcctaaagc caccccctgg cagaaacttc agctgtgtgt tctggaatac tcacgtgagg | 600 |
| gaacttactt tggccagcat tgaccttcaa agtcagatgg aacccaggac ccatccaact | 660 |
| tgg. | 663 |

In another embodiment, the costimulatory polypeptide domain of the fusion protein can have at least 80%, 85%, 90%, 95%, 99%, or 100% sequence identity to:

| (SEQ ID NO: 60) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W. | 221 |

It will be appreciated that the signal sequence will be removed in the mature protein. Additionally, it will be appreciated that signal peptides from other organisms can be used to enhance the secretion of the fusion protein from a host during manufacture. SEQ ID NO:61 provides the non-human primate amino acid sequence of SEQ ID NO:60 without the signal sequence:

| (SEQ ID NO: 61) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPFPGRN FSCVFWNTHV | 180 |
| RELTLASIDL QSQMEPRTHP TW. | 202 |

In another embodiment, the costimulatory polypeptide domain of the fusion protein includes the IgV domain of non-human primate B7-DC. The costimulatory polypeptide domain can be encoded by a nucleotide sequence having at least 80%, 85%, 90%, 95%, 99%, or 100% sequence identity to:

| (SEQ ID NO: 62) | |
|---|---|
| ttcacagtga cagtccctaa ggaactgtac ataatagagc atggcagcaa tgtgaccctg | 60 |
| gaatgcaact ttgacactgg aagtcatgtg aaccttggag caataacagc cagtttgcaa | 120 |
| aaggtggaaa atgatacatc cccacaccgt gaaagagcca ctttgctgga ggagcagctg | 180 |
| cccctaggga aggcctcgtt ccacatacct caagtccaag tgagggacga aggacagtac | 240 |
| caatgcataa tcatctatgg ggtcgcctgg gactacaagt acctgactct gaaa. | 294 |

The costimulatory polypeptide domain of the fusion protein can have at least 80%, 85%, 90%, 95%, 99%, or 100% sequence identity to:

| (SEQ ID NO: 63), also referred to as B7-DC | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW DYKYLTLK. | 98 |

d. B7 Costimulatory Extracellular Domain Fragments

It will be appreciated that B7-DC, B7-1, B7-2 and B7-H5 extracellular domains can contain one or more amino acids from the signal peptide or the putative transmembrane domain of B7-DC, 137-1, B7-2 or B7-H5. During secretion, the number of amino acids of the signal peptide that are cleaved can vary depending on the expression system and the host. Additionally, fragments of B7-DC, B7-1, B7-2 or B7-H5 extracellular domain missing one or more amino acids from the C-terminus or the N-terminus that retain the ability to bind to their natural receptors can be used as a fusion partner for the disclosed fusion proteins.

B7-DC

Exemplary suitable fragments of murine B7-DC that can be used as a costimulatory polypeptide domain include, but are not limited to, the following:
24-221, 24-220, 24-219, 24-218, 24-217, 24-216, 24-215, 23-221, 23-220, 23-219, 23-218, 23-217, 23-216, 23-215, 22-221, 22-220, 22-219, 22-218, 22-217, 22-216, 22-215, 21-221, 21-220, 21-219, 21-218, 21-217, 21-216, 21-215, 20-221, 20-220, 20-219, 20-218, 20-217, 20-216, 20-215, 19-221, 19-220, 19-219, 19-218, 19-217, 19-216, 19-215, 18-221, 18-220, 18-219, 18-218, 18-217, 18-216, 18-215, 17-221, 17-220, 17-219, 17-218, 17-217, 17-216, 17-215, 16-221, 16-220, 16-219, 16-218, 16-217, 16-216, 16-215,

### of SEQ ID NO:80.

Additional suitable fragments of murine B7-DC include, but are not limited to, the following:
20-221, 33-222, 33-223, 33-224, 33-225, 33-226, 33-227, 21-221, 21-222, 21-223, 21-224, 21-225, 21-226, 21-227, 22-221, 22-222, 22-223, 22-224, 22-225, 22-226, 22-227, 23-221, 23-222, 23-223, 23-224, 23-225, 23-226, 23-227, 24-221, 24-222, 24-223, 24-224, 24-225, 24-226, 24-227,
   of SEQ ID NO:1, optionally with one to five amino acids of a signal peptide attached to the N-terminal end. The signal peptide may be any disclosed herein, including the signal peptide contained within SEQ ID NO:1, or may be any signal peptide known in the art.

Exemplary suitable fragments of human B7-DC that can be used as a costimulatory polypeptide domain include, but are not limited to, the following:
24-221, 24-220, 24-219, 24-218, 24-217, 24-216, 24-215, 23-221, 23-220, 23-219, 23-218, 23-217, 23-216, 23-215, 22-221, 22-220, 22-219, 22-218, 22-217, 22-216, 22-215, 21-221, 21-220, 21-219, 21-218, 21-217, 21-216, 21-215, 20-221, 20-220, 20-219, 20-218, 20-217, 20-216, 20-215, 19-221, 19-220, 19-219, 19-218, 19-217, 19-216, 19-215, 18-221, 18-220, 18-219, 18-218, 18-217, 18-216, 18-215, 17-221, 17-220, 17-219, 17-218, 17-217, 17-216, 17-215, 16-221, 16-220, 16-219, 16-218, 16-217, 16-216, 16-215,

### of SEQ ID NO:83.

Additional suitable fragments of human B7-DC include, but are not limited to, the following:
20-221, 33-222, 33-223, 33-224, 33-225, 33-226, 33-227, 21-221, 21-222, 21-223, 21-224, 21-225, 21-226, 21-227, 22-221, 22-222, 22-223, 22-224, 22-225, 22-226, 22-227, 23-221, 23-222, 23-223, 23-224, 23-225, 23-226, 23-227, 24-221, 24-222, 24-223, 24-224, 24-225, 24-226, 24-227,
   of SEQ ID NO:3, optionally with one to five amino acids of a signal peptide attached to the N-terminal end. The signal peptide may be any disclosed herein, including the signal peptide contained within SEQ ID NO:3, or may be any signal peptide known in the art.

Exemplary suitable fragments of non-human primate B7-DC that can be used as a costimulatory polypeptide domain include, but are not limited to, the following:
24-221, 24-220, 24-219, 24-218, 24-217, 24-216, 24-215, 23-221, 23-220, 23-219, 23-218, 23-217, 23-216, 23-215, 22-221, 22-220, 22-219, 22-218, 22-217, 22-216, 22-215, 21-221, 21-220, 21-219, 21-218, 21-217, 21-216, 21-215, 20-221, 20-220, 20-219, 20-218, 20-217, 20-216, 20-215, 19-221, 19-220, 19-219, 19-218, 19-217, 19-216, 19-215, 18-221, 18-220, 18-219, 18-218, 18-217, 18-216, 18-215, 17-221, 17-220, 17-219, 17-218, 17-217, 17-216, 17-215, 16-221, 16-220, 16-219, 16-218, 16-217, 16-216, 16-215,

### of SEQ ID NO:86.

Additional suitable fragments of non-human primate B7-DC include, but are not limited to, the following:
20-221, 33-222, 33-223, 33-224, 33-225, 33-226, 33-227, 21-221, 21-222, 21-223, 21-224, 21-225, 21-226, 21-227, 22-221, 22-222, 22-223, 22-224, 22-225, 22-226, 22-227, 23-221, 23-222, 23-223, 23-224, 23-225, 23-226, 23-227, 24-221, 24-222, 24-223, 24-224, 24-225, 24-226, 24-227,
   of SEQ ID NO:5, optionally with one to five amino acids of a signal peptide attached to the N-terminal end. The signal peptide may be any disclosed herein, including the signal peptide contained within SEQ ID NO:5, or may be any signal peptide known in the art.

B7-1

Exemplary suitable fragments of murine B7-1 that can be used as a costimulatory polypeptide domain include, but are not limited to, the following:
42-246, 42-245, 42-244, 42-243, 42-242, 42-241, 42-240, 41-246, 41-245, 41-244, 41-243, 41-242, 41-241, 41-240, 40-246, 40-245, 40-244, 40-243, 40-242, 40-241, 40-240, 39-246, 39-245, 39-244, 39-243, 39-242, 39-241, 39-240, 38-246, 38-245, 38-244, 38-243, 38-242, 38-241, 38-240, 37-246, 37-245, 37-244, 37-243, 37-242, 37-241, 37-240, 36-246, 36-245, 36-244, 36-243, 36-242, 36-241, 36-240, 35-246, 35-245, 35-244, 35-243, 35-242, 35-241, 35-240, 34-246, 34-245, 34-244, 34-243, 34-242, 34-241, 34-240,

### of SEQ ID NO:89.

Additional suitable fragments of murine B7-1 include, but are not limited to, the following:
38-246, 38-247, 38-248, 38-249, 38-250, 38-251, 38-252, 39-246, 39-247, 39-248, 39-249, 39-250, 39-251, 39-252, 40-246, 40-247, 40-248, 40-249, 40-250, 40-251, 40-252, 41-246, 41-247, 41-248, 41-249, 41-250, 41-251, 41-252, 42-246, 42-247, 42-248, 42-249, 42-250, 42-251, 42-252,
   of SEQ ID NO:7, optionally with one to five amino acids of a signal peptide attached to the N-terminal end. The signal peptide may be any disclosed herein, including the signal peptide contained within SEQ ID NO:7, or may be any signal peptide known in the art.

Exemplary suitable fragments of human 87-1 that can be used as a costimulatory polypeptide domain include, but are not limited to, the following:
39-243, 39-242, 39-241, 39-240, 39-239, 39-238, 39-237, 38-243, 38-242, 38-241, 38-240, 38-239, 38-238, 38-237, 37-243, 37-242, 37-241, 37-240, 37-239, 37-238, 37-237, 36-243, 36-242, 36-241, 36-240, 36-239, 36-238, 36-237, 35-243, 35-242, 35-241, 35-190, 35-239, 35-238, 35-237, 34-243, 34-242, 34-241, 34-240, 34-239, 34-238, 34-237, 33-243, 33-242, 33-241, 33-240, 33-239, 33-238, 33-237, 32-243, 32-242, 32-241, 32-240, 32-239, 32-238, 32-237, 31-243, 31-242, 31-241, 31-240, 31-239, 31-238, 31-237,

### of SEQ ID NO:92.

Additional suitable fragments of human B7-1 include, but are not limited to, the following:
35-243, 35-244, 35-245, 35-246, 35-247, 35-248, 35-249, 36-243, 36-244, 36-245, 36-246, 36-247, 36-248, 36-249, 37-243, 37-244, 37-245, 37-246, 37-247, 37-248, 37-249, 38-243, 38-244, 38-245, 38-246, 38-247, 38-248, 38-249, 39-243, 39-244, 39-245, 39-246, 39-247, 39-248, 39-249,
   of SEQ ID NO:9, optionally with one to five amino acids of a signal peptide attached to the N-terminal end. The signal peptide may be any disclosed herein, including the signal peptide contained within SEQ ID NO:9, or may be any signal peptide known in the art.

B7-2

Exemplary suitable fragments of murine B7-2 that can be used as a costimulatory polypeptide domain include, but are not limited to, the following:
28-244, 28-243, 28-242, 28-241, 28-240, 28-239, 28-238, 27-244, 27-243, 27-242, 27-241, 27-240, 27-239, 27-238, 26-244, 26-243, 26-242, 26-241, 26-240, 26-239, 26-238, 25-244, 25-243, 25-242, 25-241, 25-240, 25-239, 25-238, 24-244, 24-243, 24-242, 24-241, 24-240, 24-239, 24-238, 23-244, 23-243, 23-242, 23-241, 23-240, 23-239, 23-238, 22-244, 22-243, 22-242, 22-241, 22-240, 22-239, 22-238, 21-244, 21-243, 21-242, 21-241, 21-240, 21-239, 21-238, 20-244, 20-243, 20-242, 20-241, 20-240, 20-239, 20-238,

### of SEQ ID NO:95.

Additional suitable fragments of murine B7-2 include, but are not limited to, the following:
24-244, 24-245, 24-246, 24-247, 24-248, 24-249, 24-250, 25-244, 25-245, 25-246, 25-247, 25-248, 25-249, 25-250, 26-244, 26-245, 26-246, 26-247, 26-248, 26-249, 26-250, 27-244, 27-245, 27-246, 27-247, 27-248, 27-249, 27-250, 28-244, 28-245, 28-246, 28-247, 28-248, 28-249, 28-250,
   of SEQ ID NO:11, optionally with one to five amino acids of a signal peptide attached to the N-terminal end. The signal peptide may be any disclosed herein, including the signal peptide contained within SEQ ID NO:11, or may be any signal peptide known in the art.

Exemplary suitable fragments of human B7-2 that can be used as a costimulatory polypeptide domain include, but are not limited to, the following:
28-247, 28-246, 28-245, 28-244, 28-243, 28-242, 28-241, 27-247, 27-246, 27-245, 27-244, 27-243, 27-242, 27-241, 26-247, 26-246, 26-245, 26-244, 26-243, 26-242, 26-241, 25-247, 25-246, 25-245, 25-244, 25-243, 25-242, 25-241, 24-247, 24-246, 24-245, 24-244, 24-243, 24-242, 24-241, 23-247, 23-246, 23-245, 23-244, 23-243, 23-242, 23-241, 22-247, 22-246, 22-245, 22-244, 22-243, 22-242, 22-241, 21-247, 21-246, 21-245, 21-244, 21-243, 21-242, 21-241, 20-247, 20-246, 20-245, 20-244, 20-243, 20-242, 20-241,

### of SEQ ID NO:98.

Additional suitable fragments of human B7-2 include, but are not limited to, the following:
24-247, 24-248, 24-249, 24-250, 24-251, 24-252, 24-253, 25-247, 25-248, 25-249, 25-250, 25-251, 25-252, 25-253, 26-247, 26-248, 26-249, 26-250, 26-251, 26-252, 26-253, 27-247, 27-248, 27-249, 27-250, 27-251, 27-252, 27-253, 28-247, 28-248, 28-249, 28-250, 28-251, 28-252, 28-253,
   of SEQ ID NO:13, optionally with one to five amino acids of a signal peptide attached to the N-terminal end. The signal peptide may be any disclosed herein, including the signal peptide contained within SEQ ID NO:13, or may be any signal peptide known in the art.

B7-H5

Exemplary suitable fragments of murine B7-H5 that can be used as a costimulatory polypeptide domain include, but are not limited to, the following:
37-190, 37-189, 37-188, 37-187, 37-186, 37-185, 37-184, 36-190, 36-189, 36-188, 36-187, 36-186, 36-185, 36-184, 35-190, 35-189, 35-188, 35-187, 35-186, 35-185, 35-184, 34-190, 34-189, 34-188, 34-187, 34-186, 34-185, 34-184, 33-190, 33-189, 33-188, 33-187, 33-186, 33-185, 33-184, 32-190, 32-189, 32-188, 32-187, 32-186, 32-185, 32-184, 31-190, 31-189, 31-188, 31-187, 31-186, 31-185, 31-184, 30-190, 30-189, 30-188, 30-187, 30-186, 30-185, 30-184, 29-190, 29-189, 29-188, 29-187, 29-186, 29-185, 29-184,

### of SEQ ID NO:101.

Additional suitable fragments of murine B7-H5 include, but are not limited to, the following:
33-190, 33-191, 33-192, 33-193, 33-194, 33-195, 33-196, 34-190, 34-191, 34-192, 34-193, 34-194, 34-195, 34-196, 35-190, 35-191, 35-192, 35-193, 35-194, 35-195, 35-196, 36-190, 36-191, 36-192, 36-193, 36-194, 36-195, 36-196, 37-190, 37-191, 37-192, 37-193, 37-194, 37-195, 37-196,
   of SEQ ID NO:15, optionally with one to five amino acids of a signal peptide attached to the N-terminal end. The signal peptide may be any disclosed herein, including the signal peptide contained within SEQ ID NO:15, or may be any signal peptide known in the art.

Exemplary suitable fragments of human B7-H5 that can be used as a costimulatory polypeptide domain include, but are not limited to, the following:
37-193, 37-192, 37-191, 37-190, 37-189, 37-188, 37-187, 36-193, 36-192, 36-191, 36-190, 36-189, 36-188, 36-187, 35-193, 35-192, 35-191, 35-190, 35-189, 35-188, 35-187, 34-193, 34-192, 34-191, 34-190, 34-189, 34-188, 34-187, 33-193, 33-192, 33-191, 33-190, 33-189, 33-188, 33-187, 32-193, 32-192, 32-191, 32-190, 32-189, 32-188, 32-187, 31-193, 31-192, 31-191, 31-190, 31-189, 31-188, 31-187, 30-193, 30-192, 30-191, 30-190, 30-189, 30-188, 30-187, 29-193, 29-192, 29-191, 29-190, 29-189, 29-188, 29-187,

### of SEQ ID NO:104.

Additional suitable fragments of human B7-H5 include, but are not limited to, the following:
33-193, 33-194, 33-195, 33-196, 33-197, 33-198, 33-199, 34-193, 34-194, 34-195, 34-196, 34-197, 34-198, 34-199, 35-193, 35-194, 35-195, 35-196, 35-197, 35-198, 35-199, 36-193, 36-194, 36-195, 36-196, 36-197, 36-198, 36-199, 37-193, 37-194, 37-195, 37-196, 37-197, 37-198, 37-199,
   of SEQ ID NO:17, optionally with one to five amino acids of a signal peptide attached to the N-terminal end. The signal peptide may be any disclosed herein, including the signal peptide contained within SEQ ID NO:17, or may be any signal peptide known in the art.

b. Variant B7 Costimulatory Polypeptides

Variants of costimulatory molecules can also be used. In one embodiment the variant B7 costimulatory polypeptide has the same activity, substantially the same activity, or different activity as a reference B7 costimulatory polypeptide, for example a non-mutated B7-DC polypeptide. Substantially the same activity means it retains the ability to costimulate T cells.

Exemplary variant B7 co-stimulatory polypeptides include, but are not limited to B7-1, B7-2, B7-H5 or B7-DC polypeptides that are mutated to contain a deletion, substitution, insertion, or rearrangement of one or more amino acids. A variant B7 costimulatory polypeptide can have any combination of amino acid substitutions, deletions or insertions. In one embodiment, isolated B7 variant polypeptides have an integer number of amino acid alterations such that their amino acid sequence shares at least 60, 70, 80, 85, 90, 95, 97, 98, 99, 99.5 or 100% identity with an amino acid sequence of a wild type B7 co-stimulatory polypeptide. In a preferred embodiment, B7 variant polypeptides have an amino acid sequence sharing at least 60, 70, 80, 85, 90, 95, 97, 98, 99, 99.5 or 100% identity with the amino acid sequence of a wild type murine or wild type human B7 polypeptide (GenBank Accession Number NM_{—}025239, NM_{—}005191, U04343, or NP_{—}071436).

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity (the number of identical residues)/(alignment length in amino acid residues)*100. For this calculation, alignment length includes internal gaps but does not include terminal gaps.

Amino acid substitutions in B7 costimulatory polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gin); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

B7 family molecules are expressed at the cell surface with a membrane proximal constant IgC domain and a membrane distal IgV domain. Receptors for these ligands share a common extracellular IgV-like domain. Interactions of receptor-ligand pairs are mediated predominantly through residues in the IgV domains of the ligands and receptors. In general, IgV domains are described as having two sheets that each contain a layer of β-strands. These β-strands are referred to as A′, B, C, C′, C″, D, E, F and G. In one embodiment the B7 variant polypeptides contain amino acid alterations (i.e., substitutions, deletions or insertions) within one or more of these β-strands in any possible combination. In another embodiment, B7 variants contain one or more amino acid alterations (i.e., substitutions, deletions or insertions) within the A′, C, C′, C″, D, E, F or G β-strands. In a preferred embodiment B7 variants contain one or more amino acid alterations in the G β-strand.

An exemplary variant B7-DC co-stimulatory polypeptide is one that is mutated so that it retains its ability to enhance T cell activity, but shows reduced PD-1 binding activity. Accordingly, with respect to murine human or non-human primate B7-DC co-stimulatory polypeptides, a variant 87-DC polypeptide can contain, without limitation, substitutions, deletions or insertions at position 33 of the A′ β-strand, positions 39 or 41 of the B β-strand, positions 56 or 58 of the C β-strand, positions 65 or 67 of the C′ β-strand, positions 71 or 72 of the C″ β-strand, position 84 of the D β-strand, position 88 of the E β-strand, positions 101, 103 or 105 of the F β-strand, or positions 110, 111, 113 or 116 of the G β-strand. These amino acid positions are relative to the full length amino acid sequences of murine and human B7-DC provided by SEQ ID NO:1 and SEQ ID NO:3, respectively. It will be appreciated that fragments of murine and human B7-DC polypeptides may contain substitutions, deletions or insertions at corresponding amino acid positions.

In one embodiment, variant B7-DC polypeptides contain a substitution at position 33 (e.g., a serine substitution for aspartic acid at position 33), a substitution at position 39 (e.g., a tyrosine substitution for serine at position 39), a substitution at position 41 (e.g., a serine substitution for glutamic acid at position 41), a substitution at position 56 (e.g., a serine substitution for arginine at position 56), a substitution at position 58 (e.g., a tyrosine substitution for serine at position 58), a substitution at position 65 (e.g., a serine substitution for aspartic acid at position 65), a substitution at position 67 (e.g., a tyrosine substitution for serine at position 67), a substitution at position 71 (e.g., a serine substitution for glutamic acid at position 71), a substitution at position 72 (e.g., a serine substitution for arginine at position 72), a substitution at position 84 (e.g., a serine substitution for lysine at position 84), a substitution at position 88 (e.g., an alanine substitution for histidine at position 88), a substitution at position 101 (e.g., a serine substitution for arginine at position 101), a substitution at position 103 (e.g., an alanine substitution for leucine at position 103), a substitution at position 105 (e.g., an alanine substitution for isoleucine at position 105), a substitution at position 110 (e.g., an alanine substitution for tryptophan at position 110), a substitution at position 111 (e.g., a serine substitution for aspartic acid at position 111), a substitution at position 113 (e.g., a serine substitution for lysine at position 113), or a substitution at position 116 (e.g., a tyrosine substitution for threonine at position 116).

It is understood, however, that substitutions at the recited amino acid positions can be made using any amino acid or amino acid analog. For example, the substitutions at the recited positions can be made with any of the naturally-occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine).

In one embodiment, the costimulatory polypeptide domain of the fusion protein includes the extracellular domain of human B7-DC with a K113S substitution provided by SEQ ID NO:64, or a fragment thereof:

| (SEQ ID NO: 64) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYSYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W. | 221 |

It will be appreciated that the signal sequence will be removed in the mature protein. Additionally, it will be appreciated that signal peptides from other organisms can be used to enhance the secretion of the fusion protein from a host during manufacture. SEQ ID NO:65 provides the human amino acid sequence of SEQ ID NO:64 without the signal sequence:

| (SEQ ID NO: 65) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYSYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW. | 202 |

In another embodiment, the costimulatory polypeptide domain of the fusion protein includes the IgV domain of human B7-DC with a K113S substitution provided by SEQ ID NO:66, or a fragment thereof:

| (SEQ ID NO: 66) | |
|---|---|
| FTVTVPKELY IIEHGSNVTL ECNFDTGSHV NLGAITASLQ KVENDTSPHR ERATLLEEQL | 60 |
| PLGKASEHIP QVQVRDEGQY QCIIIYGVAW DYSYLTLK. | 98 |

In another embodiment, the costimulatory polypeptide domain of the fusion protein includes the extracellular domain of human B7-DC with a D111 S substitution provided by SEQ ID NO:67, or a fragment thereof:

| (SEQ ID NO: 67) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW SYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W. | 221 |

It will be appreciated that the signal sequence will be removed in the mature protein. Additionally, it will be appreciated that signal peptides from other organisms can be used to enhance the secretion of the fusion protein from a host during manufacture. SEQ ID NO:68 provides the human amino acid sequence of SEQ ID NO:67 without the signal sequence:

| (SEQ ID NO: 68) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WSYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW | 202. |

In another embodiment, the costimulatory polypeptide domain of the fusion protein includes the IgV domain of human B7-DC with a D111S substitution provided by SEQ ID NO:69, or a fragment thereof:

| (SEQ ID NO: 69) | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW SYKYLTLK | 98. |

While the substitutions described herein are with respect to mouse and human B7-DC, it is noted that one of ordinary skill in the art could readily make equivalent alterations in the corresponding polypeptides from other species (e.g., mouse, rat, hamster, guinea pig, gerbil, rabbit, dog, cat, horse, pig, sheep, cow or non-human primate).

It will be appreciated that nucleic acids encoding the disclosed fusion polypeptides may be optimized for expression in the expression host of choice. Codons may be substituted with alternative codons encoding the same amino acid to account for differences in codon usage between the mammal from which the nucleic acid sequence is derived and the expression host. In this manner, the nucleic acids may be synthesized using expression host-preferred codons.

c. Properties of Variant B7 Costimulatory Polypeptides

The disclosed B7 costimulatory polypeptides and variants and fragments thereof are capable of activating T cells. The T cell response that results from the interaction typically is greater than the response in the absence of the costimulatory polypeptide. The response of the T cell in the absence of the costimulatory polypeptide can be no response or can be a response significantly lower than in the presence of the costimulatory polypeptide.

Exemplary variants of costimulatory polypeptides are those that have an insertion, deletion, or substitution of one or more amino acids that reduces or prevents the co-stimulatory molecule from participating in signal transduction pathways that transmit inhibitory signals in T cells.

Methods for measuring the binding affinity between two molecules are well known in the art. Methods for measuring the binding affinity of B7 variant polypeptides to receptors include, but are not limited to, fluorescence activated cell sorting (FACS), surface plasmon resonance, fluorescence anisotropy, affinity chromatography and affinity selection-mass spectrometry.

Methods for measuring costimulation of T cells are well known in the art and include measurements of T cell proliferation and secretion of cytokines, including, but not limited to, Il-2, IL-4, IL-5, IL-6, IL-10, IL-13, and IFN-γ. Proliferation of T cells can be measured by a number of methods including, but not limited to, cell counting, measuring DNA synthesis by uptake of labeled nucleotides (such as [³H] TdR and BrdU) and measuring metabolic activity with tetrazolium salts. Methods for measuring the secretion of cytokines include, but are not limited to, ELISA.

B. Antigen-Binding Targeting Domain

The fusion proteins also contain antigen-binding targeting domains. In some embodiments, the targeting domains bind to antigens, ligands or receptors that are specific to tumor cells or tumor-associated neovasculature, or are upregulated in tumor cells or tumor-associated neovasculature compared to normal tissue. In some embodiments, the targeting domains bind to antigens, ligands or receptors that are specific to immune tissue involved in the regulation of T cell activation in response to infectious disease causing agents.

1. Tumor/Tumor-Associated Vasculature Targeting Domains

a. Antigens, Ligands and Receptors to Target

i. Tumor-Specific and Tumor-Associated Antigens

In one embodiment the fusion proteins contain a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(413):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Ace. Nos. X03363 and M17730), HER3 (GenBank Ace. Nos. U29339 and M34309), HER4 (Plowman, et al., Nature, 366:473 (1993); GenBank Ace. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-11 (GenBank Ace. Nos. X03562, X00910, M17863 and M17862), transferrin receptor (Trowbridge and Omary, Proc. Nat. Acad. USA, 78:3039 (1981); GenBank Ace. Nos. X01060 and M11507), estrogen receptor (GenBank Ace. Nos. M38651, X03635, X99101, U47678 and M12674), progesterone receptor (GenBank Ace. Nos. X51730, X69068 and M15716), follicle stimulating hormone receptor (FSH-R) (GenBank Ace. Nos. Z34260 and M65085), retinoic acid receptor (GenBank Ace. Nos. L12060, M60909, X77664, X57280, X07282 and X06538), MUC-1 (Barnes, et al., Proc. Nat. Acad. Sci. USA, 86:7159 (1989); GenBank Ace. Nos. M65132 and M64928) NY-ESO-1 (GenBank Ace. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Ace. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Ace. No. 573003, Adema, et al., J. Biol. Chem., 269:20126 (1994)), MAGE (van den Bruggen, et al., Science, 254:1643 (1991)); GenBank Ace. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Ace. No. U19180; U.S. Pat. Nos. 5,683,886 and 5,571,711), GAGE (GenBank Ace. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Ace. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT3S); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, RAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

ii. Antigens Associated with Tumor Neovasculature

Protein therapeutics can be ineffective in treating tumors because they are inefficient at tumor penetration. Tumor-associated neovasculature provides a readily accessible route through which protein therapeutics can access the tumor. In another embodiment the fusion proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

iii. Chemokines/Chemokine Receptors

In another embodiment, the fusion proteins contain a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups namely CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

2. Molecular Classes of Targeting Domains

a. Ligands and Receptors

In one embodiment, tumor or tumor-associated neovasculature targeting domains are ligands that bind to cell surface antigens or receptors that are specifically expressed on tumor cells or tumor-associated neovasculature or are overexpressed on tumor cells or tumor-associated neovasculature as compared to normal tissue. Tumors also secrete a large number of ligands into the tumor microenvironment that affect tumor growth and development. Receptors that bind to ligands secreted by tumors, including, but not limited to growth factors, cytokines and chemokines, including the chemokines provided above, are suitable for use in the disclosed fusion proteins. Ligands secreted by tumors can be targeted using soluble fragments of receptors that bind to the secreted ligands. Soluble receptor fragments are fragments polypeptides that may be shed, secreted or otherwise extracted from the producing cells and include the entire extracellular domain, or fragments thereof.

b. Single Polypeptide Antibodies

In another embodiment, tumor or tumor-associated neovasculature targeting domains are single polypeptide antibodies that bind to cell surface antigens or receptors that are specifically expressed on tumor cells or tumor-associated neovasculature or are overexpressed on tumor cells or tumor-associated neovasculature as compared to normal tissue. Single domain antibodies are described above with respect to coinhibitory receptor antagonist domains.

c. Fc Domains

In another embodiment, tumor or tumor-associated neovasculature targeting domains are Fc domains of immunoglobulin heavy chains that bind to Fc receptors expressed on tumor cells or on tumor-associated neovasculature. The Fc region as used herein includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human or murine immunoglobulin. In a more preferred embodiment, the Fc domain is derived from human IgG1 or murine IgG2a including the C_{H}2 and C_{H}3 regions.

In one embodiment, the hinge, C_{H}2 and C_{H}3 regions of a human immunoglobulin Cγ1 chain are encoded by a nucleic acid having at least 80%, 85%, 90%, 95%, 99% or 100% sequence identity to:

| (SEQ ID NO: 70) | |
|---|---|
| gagcctaagt catgtgacaa gacccatacg tgcccaccct gtcccgctcc agaactgctg | 60 |
| gggggaccta gcgttttctt gttcccccca aagcccaagg acaccctcat gatctcacgg | 120 |
| actcccgaag taacatgcgt agtagtcgac gtgagccacg aggatcctga agtgaagttt | 180 |
| aattggtacg tggacggagt cgaggtgcat aatgccaaaa ctaaacctcg ggaggagcag | 240 |
| tataacagta cctaccgcgt ggtatccgtc ttgacagtgc tccaccagga ctggctgaat | 300 |
| ggtaaggagt ataaatgcaa ggtcagcaac aaagctcttc ccgccccaat tgaaaagact | 360 |
| atcagcaagg ccaagggaca accccgcgag ccccaggttt acacccttcc accttcacga | 420 |
| gacgagctga ccaagaacca ggtgtctctg acttgtctgg tcaaaggttt ctatccttcc | 480 |
| gacatcgcag tggagtggga gtcaaacggg cagcctgaga ataactacaa gaccacaccc | 540 |
| ccagtgcttg atagcgatgg gagctttttc ctctacagta agctgactgt ggacaaatcc | 600 |
| cgctggcagc agggaaacgt tttctcttgt agcgtcatgc atgaggccct ccacaaccat | 660 |
| tatactcaga aaagcctgag tctgagtccc ggcaaa | 696 |

The hinge, C_{H}2 and C_{H}3 regions of a human immunoglobulin Cy1 chain encoded by SEQ ID NO:70 has the following amino acid sequence:

| (SEQ ID NO: 71) | |
|---|---|
| EPKSCDKTHT CPPCPAPELL GGPSVFLFPP KPKDTLMISR TPEVTCVVVD VSHEDPEVKF | 60 |
| NWYVDGVEVH NAKTKPREEQ YNSTYRVVSV LTVLHQDWLN GKEYKCKVSN KALPAPIEKT | 120 |
| ISKAKGQPRE PQVYTLPPSR DELTKQVSL TCLVKGFYPS DIAVEWESNG QPENNYKTTP | 180 |
| PVLDSDGSFF LYSKLTVDKS RWQQGNVFSC SVMHEALHNH YTQKSLSLSP GK | 232 |

In another embodiment, the hinge, C_{H}2 and C_{H}3 regions of a murine immunoglobulin Cγ2a chain are encoded by a nucleic acid having at least 80%, 85%, 90%, 95%, 99% or 100% sequence identity to:

| (SEQ ID NO: 72) | |
|---|---|
| gagccaagag gtcctacgat caagccctgc ccgccttgta aatgcccagc tccaaatttg | 60 |
| ctgggtggac cgtcagtctt tatcttcccg ccaaagataa aggacgtctt gatgattagt | 120 |
| ctgagcccca tcgtgacatg cgttgtggtg gatgtttcag aggatgaccc cgacgtgcaa | 180 |
| atcagttggt tcgttaacaa cgtggaggtg cataccgctc aaacccagac ccacagagag | 240 |
| gattataaca gcaccctgcg ggtagtgtcc gccctgccga tccagcatca ggattggatg | 300 |
| agcgggaaag agttcaagtg taaggtaaac aacaaagatc tgccagcgcc gattgaacga | 360 |
| accattagca agccgaaagg gagcgtgcgc gcacctcagg tttacgtcct tcctccacca | 420 |
| gaagaggaga tgacgaaaaa gcaggtgacc ctgacatgca tggtaactga ctttatgcca | 480 |
| gaagatattt acgtggaatg gactaataac ggaaagacag agctcaatta caagaacact | 540 |
| gagcctgttc tggattctga tggcagctac tttatgtact ccaaattgag ggtcgagaag | 600 |
| aagaattggg tcgagagaaa cagttatagt tgctcagtgg tgcatgaggg cctccataat | 660 |
| catcacacca caaagtcctt cagccgaacg cccgggaaa | 699 |

The hinge, C_{H}2 and C_{H}3 regions of a murine immunoglobulin Cγ2a chain encoded by SEQ ID NO:72 has the following amino acid sequence:

| (SEQ ID NO: 73) | |
|---|---|
| EPRGPTIKPC PPCKCPAPNL LGGPSVFIFP PKIKDVLMIS LSPIVTCVVV DVSEDDPDVQ | 60 |
| ISWFVNNVEV HTAQTQTHRE DYNSTLRVVS ALPIQHQDWM SGKEFKCKVN NKDLPAPIER | 120 |
| TISKPKGSVR APQVYVLPPP EEEMTKKQVT LTCMVTDFMP EDIYVEWTNN GKTELNYKNT | 180 |
| EPVLDSDGSY FMYSKLRVEK KNWVERNSYS CSVVHEGLHN HHTTKSFSRT PGK | 233 |

In one embodiment, the Fc domain may contain one or more amino acid insertions, deletions or substitutions that enhance binding to specific Fc receptors that specifically expressed on tumors or tumor-associated neovasculature or are overexpressed on tumors or tumor-associated neovasculature relative to normal tissue. Suitable amino acid substitutions include conservative and non-conservative substitutions, as described above.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fey receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA). In a preferred embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F243L, R929P, Y300L, V3051 or P296L substitutions. These amino acid substitutions may be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution.

d. Glycophosphatidylinositol Anchor Domain

In another embodiment, tumor or tumor-associated neovasculature targeting domains are polypeptides that provide a signal for the posttranslational addition of a glycosylphosphatidylinositol (GPI) anchor. GPI anchors are glycolipid structures that are added posttranslationally to the C-terminus of many eukaryotic proteins. This modification anchors the attached protein in the outer leaflet of cell membranes. GPI anchors can be used to attach T cell receptor binding domains to the surface of cells for presentation to T cells. In this embodiment, the GPI anchor domain is C-terminal to the T cell receptor binding domain.

In one embodiment, the GPI anchor domain is a polypeptide that signals for the posttranslational addition addition of a GPI anchor when the polypeptide is expressed in a eukaryotic system. Anchor addition is determined by the GPI anchor signal sequence, which consists of a set of small amino acids at the site of anchor addition (the ω site) followed by a hydrophilic spacer and ending in a hydrophobic stretch (Low, FASEB J., 3:1600-1608 (1989)). Cleavage of this signal sequence occurs in the ER before the addition of an anchor with conserved central components (Low, FASEB J., 3:1600-1608 (1989)) but with variable peripheral moieties (Homans et al., Nature, 333:269-272 (1988)). The C-terminus of a GPI-anchored protein is linked through a phosphoethanolamine bridge to the highly conserved core glycan, mannose(α1-2)mannose(α1-6)mannose(α1-4)glucosamine(α1-6)myo-inositol. A phospholipid tail attaches the GPI anchor to the cell membrane. The glycan core can be variously modified with side chains, such as a phosphoethanolamine group, mannose, galactose, sialic acid, or other sugars. The most common side chain attached to the first mannose residue is another mannose. Complex side chains, such as the N-acetylgalactosamine-containing polysaccharides attached to the third mannose of the glycan core, are found in mammalian anchor structures. The core glucosamine is rarely modified. Depending on the protein and species of origin, the lipid anchor of the phosphoinositol ring is a diacylglycerol, an alkylacylglycerol, or a ceramide. The lipid species vary in length, ranging from 14 to 28 carbons, and can be either saturated or unsaturated. Many GPI anchors also contain an additional fatty acid, such as palmitic acid, on the 2-hydroxyl of the inositol ring. This extra fatty acid renders the GPI anchor resistant to cleavage by PI-PLC.

GPI anchor attachment can be achieved by expression of a fusion protein containing a GPI anchor domain in a eukaryotic system capable of carrying out GPI posttranslational modifications. GPI anchor domains can be used as the tumor or tumor vasculature targeting domain, or can be additionally added to fusion proteins already containing separate tumor or tumor vasculature targeting domains.

In another embodiment, GPI anchor moieties are added directly to isolated T cell receptor binding domains through an in vitro enzymatic or chemical process. In this embodiment, GPI anchors can be added to polypeptides without the requirement for a GPI anchor domain. Thus, GPI anchor moieties can be added to fusion proteins described herein having a T cell receptor binding domain and a tumor or tumor vasculature targeting domain. Alternatively, GPI anchors can be added directly to T cell receptor binding domain polypeptides without the requirement for fusion partners encoding tumor or tumor vasculature targeting domains.

C. Peptide or Polypeptide Linker Domain

Fusion proteins disclosed herein optionally contain a peptide or polypeptide linker domain that separates the costimulatory polypeptide domain from the antigen-binding targeting domain.

1. Hinge Region of Antibodies

In one embodiment, the linker domain contains the hinge region of an immunoglobulin. In a preferred embodiment, the hinge region is derived from a human immunoglobulin. Suitable human immunoglobulins that the hinge can be derived from include IgG, IgD and IgA. In a preferred embodiment, the hinge region is derived from human IgG.

In another embodiment, the linker domain contains a hinge region of an immunoglobulin as described above, and further includes one or more additional immunoglobulin domains. In one embodiment, the additional domain includes the Fc domain of an immunoglobulin. The Fc region as used herein includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human immunoglobulin. In a more preferred embodiment, the Fc domain is derived from human IgG including the C_{H}2 and C_{H}3 regions.

In another embodiment, the linker domain contains a hinge region of an immunoglobulin and either the C_{H}1 domain of an immunoglobulin heavy chain or the C_{L} domain of an immunoglobulin light chain. In a preferred embodiment, the C_{H}1 or C_{L} domain is derived from a human immunoglobulin. The C_{L} domain may be derived from either a K light chain or a 2 light chain. In a more preferred embodiment, the C_{H}1 or C_{L} domain is derived from human IgG.

Amino acid sequences of immunoglobulin hinge regions and other domains are well known in the art.

2. Other Peptide/Polypeptide Linker Domains

Other suitable peptide/polypeptide linker domains include naturally occurring or non-naturally occurring peptides or polypeptides. Peptide linker sequences are at least 2 amino acids in length. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser, Gly-Ser-Gly-Ser (SEQ ID NO:74), Ala-Ser, Gly-Gly-Gly-Ser (SEQ ID NO:75), (Gly₄-Ser)₃ (SEQ ID NO:76), (Gly₄-Ser)₄ (SEQ ID NO:77), and (Gly₄-Ser)₄ (SEQ ID NO:78). Additional flexible peptide/polypeptide sequences are well known in the art.

D. Dimerization and Multimerization Domains

The fusion proteins disclosed herein optionally contain a dimerization or multimerization domain that functions to dimerize or multimerize two or more fusion proteins. The domain that functions to dimerize or multimerize the fusion proteins can either be a separate domain, or alternatively can be contained within one of the other domains (T cell costimulatory/coinhibitory receptor binding domain, tumor/tumor neovasculature antigen-binding domain, or peptide/polypeptide linker domain) of the fusion protein.

1. Dimerization Domains

A “dimerization domain” is formed by the association of at least two amino acid residues or of at least two peptides or polypeptides (which may have the same, or different, amino acid sequences). The peptides or polypeptides may interact with each other through covalent and/or non-covalent association(s). Preferred dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. In a preferred embodiment, the dimerization domain is the hinge region of an immunoglobulin. In this particular embodiment, the dimerization domain is contained within the linker peptide/polypeptide of the fusion protein.

Additional exemplary dimerization domain can be any known in the art and include, but not limited to, coiled coils, acid patches, zinc fingers, calcium hands, a C_{H}1-C_{L} pair, an “interface” with an engineered “knob” and/or “protruberance” as described in U.S. Pat. No. 5,821,333, leucine zippers (e.g., from jun and/or fos) (U.S. Pat. No. 5,932,448), SH2 (src homology 2), SH3 (src Homology 3) (Vidal, et al., Biochemistry, 43, 7336-44 ((2004)), phosphotyrosine binding (PTB) (Zhou, et al., Nature, 378:584-592 (1995)), WW (Sudol, Prog. Biochys. Mol. Bio., 65:113-132 (1996)), PDZ (Kim, et al., Nature, 378: 85-88 (1995); Komau, et al., Science, 269:1737-1740 (1995)) 14-3-3, WD40 (Hu, et al., J Biol. Chem., 273, 33489-33494 (1998)) EH, Lim, an isoleucine zipper, a receptor dimer pair (e.g., interleukin-8 receptor (IL-8R); and integrin heterodimers such as LFA-1 and GPIIIb/IIIa), or the dimerization region(s) thereof, dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J. Biol. Chem., 269(45): 27833-27839 (1994) and Radziejewski, et al., Biochem., 32(48): 1350 (1993)) and can also be variants of these domains in which the affinity is altered. The polypeptide pairs can be identified by methods known in the art, including yeast two hybrid screens. Yeast two hybrid screens are described in U.S. Pat. Nos. 5,283,173 and 6,562,576, both of which are herein incorporated by reference in their entireties. Affinities between a pair of interacting domains can be determined using methods known in the art, including as described in Katahira, et al., J. Biol. Chem., 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

2. Multimerization Domains

A “multimerization domain” is a domain that causes three or more peptides or polypeptides to interact with each other through covalent and/or non-covalent association(s). Suitable multimerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

The coiled coil domain may be derived from laminin. In the extracellular space, the heterotrimeric coiled coil protein laminin plays an important role in the formation of basement membranes. Apparently, the multifunctional oligomeric structure is required for laminin function. Coiled coil domains may also be derived from the thrombospondins in which three (TSP-1 and TSP-2) or five (TSP-3, TSP-4 and TSP-5) chains are connected, or from COMP (COMPcc) (Guo, et at., EMBO J., 1998, 17: 5265-5272) which folds into a parallel five-stranded coiled coil (Malashkevich, et al., Science, 274: 761-765 (1996)).

Additional coiled-coil domains derived from other proteins, and other domains that mediate polypeptide multimerization are known in the art and are suitable for use in the disclosed fusion proteins.

E. Exemplary Fusion Proteins

B7-DC

A representative murine B7-DC fusion protein is encoded by a nucleic acid having at least 80%, 85%, 90%, 95%, 99% or 100% sequence identity to:

| (SEQ ID NO: 79) | |
|---|---|
| atgctgctcc tgctgccgat actgaacctg agcttacaac ttcatcctgt agcagcttta | 60 |
| ttcaccgtga cagcccctaa agaagtgtac accgtagacg tcggcagcag tgtgagcctg | 120 |
| gagtgcgatt ttgaccgcag agaatgcact gaactggaag ggataagagc cagtttgcag | 180 |
| aaggtagaaa atgatacgtc tctgcaaagt gaaagagcca ccctgctgga ggagcagctg | 240 |
| cccctgggaa aggctttgtt ccacatccct agtgtccaag tgagagattc cgggcagtac | 300 |
| cgttgcctgg tcatctgcgg ggccgcctgg gactacaagt acctgacggt gaaagtcaaa | 360 |
| gcttcttaca tgaggataga cactaggatc ctggaggttc caggtacagg ggaggtgcag | 420 |
| cttacctgcc aggctagagg ttatccccta gcagaagtgt cctggcaaaa tgtcagtgtt | 480 |
| cctgccaaca ccagccacat caggaccccc gaaggcctct accaggtcac cagtgttctg | 540 |
| cgcctcaagc ctcagcctag cagaaacttc agctgcatgt tctggaatgc tcacatgaag | 600 |
| gagctgactt cagccatcat tgaccctctg agtcggatgg aacccaaagt ccccagaacg | 660 |
| tgggagccaa gaggtcctac gatcaagccc tgcccgcctt gtaaatgccc agctccaaat | 720 |
| ttgctgggtg gaccgtcagt ctttatcttc ccgccaaaga taaaggacgt cttgatgatt | 780 |
| agtctgagcc ccatcgtgac atgcgttgtg gtggatgttt cagaggatga ccccgacgtg | 840 |
| caaatcagtt ggttcgttaa caacgtggag gtgcataccg ctcaaaccca gacccacaga | 900 |
| gaggattata acagcaccct gcgggtagtg tccgccctgc cgatccagca tcaggattgg | 960 |
| atgagcggga aagagttcaa gtgtaaggta aacaacaaag atctgccagc gccgattgaa | 1020 |
| cgaaccatta gcaagccgaa agggagcgtg cgcgcacctc aggtttacgt ccttcctcca | 1080 |
| ccagaagagg agatgacgaa aaagcaggtg accctgacat gcatggtaac tgactttatg | 1140 |
| ccagaagata tttacgtgga atggactaat aacggaaaga cagagctcaa ttacaagaac | 1200 |
| actgagcctg ttctggattc tgatggcagc tactttatgt actccaaatt gagggtcgag | 1260 |
| aagaagaatt gggtcgagag aaacagttat agttgctcag tggtgcatga gggcctccat | 1320 |
| aatcatcaca ccacaaagtc cttcagccga acgcccggga aatga | 1365 |

The murine B7-DC fusion protein encoded by SEQ ID NO:79 has the following amino acid sequence:

| (SEQ ID NO: 80) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT WEPRGPTIKP CPPCKCPAPN | 240 |
| LLGGPSVFIF PPKIKDVIMI SLSPIVTCVV VDVSEDDPDV QISWFVNNVE VHTAQTQTHR | 300 |
| EDYNSTLRVV SALPIQHQDW MSGKEFKCKV NNKDLPAPIE RTISKPKGSV RAPQVYVLPP | 360 |
| PEEEMTKKQV TLTCMVTDFM PEDIYVEWTN NGKTELNYKN TEPVLDSDGS YFMYSKLRVE | 420 |
| KKNWVERNSY SCSVVHEGLH NHHTTKSFSR TPGK | 454 |

The amino acid sequence of the murine B7-DC fusion protein of SEQ ID NO:80 without the signal sequence is:

| (SEQ ID NO: 81) | |
|---|---|
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TWEPRGPTIK PCPPCKCPAP NLLGGPSVFI FPPKIKDVLM | 240 |
| ISLSPIVTCV VVDVSEDDPD VQISWFVNNV EVHTAQTQTH REDYNSTLRV VSALPIQHQD | 300 |
| WMSGKEFKCK VNNKDLPAPI ERTISRPKGS VRAPQVYVLP PPEEEMTKKQ VTLTCMVTDF | 360 |
| MPEDIYVEWT NNGKTELNYK NTEPVLDSDG SYFMYSKLRV EKKNWVERNS YSCSVVHEGL | 420 |
| HNHHTTKSFS RTPGK | 435. |

A representative human 137-DC fusion protein is encoded by a nucleic acid having at least 80%, 85%, 90%, 95%, 99% or 100% sequence identity to:

| (SEQ ID NO: 82) | |
|---|---|
| atgatctttc ttctcttgat gctgtctttg gaattgcaac ttcaccaaat cgcggocctc | 60 |
| tttactgtga ccgtgccaaa agaactgtat atcattgagc acgggtccaa tgtgaccctc | 120 |
| gaatgtaact ttgacaccgg cagccacgtt aacctggggg ccatcactgc cagcttgcaa | 180 |
| aaagttgaaa acgacacttc acctcaccgg gagagggcaa ccctcttgga ggagcaactg | 240 |
| ccattgggga aggcctcctt tcatatccct caggtgcagg ttcgggatga gggacagtac | 300 |
| cagtgcatta ttatctacgg cgtggcttgg gattacaagt atctgaccct gaaggtgaaa | 360 |
| gcgtcctato ggaaaattaa cactcacatt cttaaggtgc cagagacgga cgaggtggaa | 420 |
| ctgacatgcc aagccaccgg ctacccgttg gcagaggtca gctggcccaa cgtgagcgta | 480 |
| cctgctaaca cttctcattc taggacaccc gagggcctct accaggttac atccgtgctc | 540 |
| cgcctcaaac cgcccccagg ccggaatttt agttgcgtgt tttggaatac ccacgtgcga | 600 |
| gagctgactc ttgcatctat tgatctgcag tcccagatgg agccacggac tcatccaact | 660 |
| tgggaaccta aatcttgcga taaaactcat acctgtcccc cttgcccagc ccccgagctt | 720 |
| ctgggaggtc ccagtgtgtt tctgtttccc ccaaaaccta aggacacact tatgatatcc | 780 |
| cgaacgccgg aagtgacatg cgtggttgtg gacgtctcac acgaagaccc ggaggtgaaa | 840 |
| ttcaactggt acgttgacgg agttgaggtt cataacgcta agaccaagcc cagagaggag | 900 |
| caatacaatt ccacctatcg agtggttagt gtactgaccg ttttgcacca agactggctg | 960 |
| aatggaaaag aatacaagtg caaagtatca aacaaggctt tgcctgcacc catcgagaag | 1020 |
| acaatttcta aagccaaagg gcagcccagg gaaccgcagg tgtacacact cccaccatcc | 1080 |
| cgcgacgagc tgacaaagaa tcaagtatcc ctgacutgoc tggtgaaagg cttttaccca | 1140 |
| tctgacattg ccgtggaatg ggaatcaaat ggacaacctg agaacaacta caaaaccact | 1200 |
| ccacctgtgc ttgacagcga cgggtccttt ttcctgtaca gtaagctcac tgtcgataag | 1260 |
| tctcgctggc agcagggcaa cgtcttttca tgtagtgtga tgcacgaagc tctgcacaac | 1320 |
| cattacaccc agaagtctct gtcactgagc ccaggtaaat ga | 1362 |

The human B7-DC fusion protein encoded by SEQ ID NO:82 has the following amino acid sequence:

| (SEQ ID NO: 83) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WEPKSCDKTH TCPPCPAPEL | 240 |
| LGGPSVFLFP PKPKDTLMIS RTPEVTCVVV DVSHEDPEVK FNWYVDGVEV HNAKTKPREE | 300 |
| QYNSTYRVVS VLTVLHQDWL NGKEYKCKVS NKALPAPIEK TISKAKGQPR EPQVYTLPPS | 360 |
| RDELTKNQVS LTCLVKGFYP SDIAVEWESN GQPENNYKTT PPVLDSDGSF FLYSKLTVDK | 420 |
| SRWQQGNVES CSVMHEALHN HYTQKSLSLS PGK | 453 |

The amino acid sequence of the human B7-DC fusion protein of SEQ ID NO:83 without the signal sequence is:

| (SEQ ID NO: 84) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVENNTHV | 180 |
| RELTLASIDL QSQMEPRTHP TWEPKSCDKT RTCPPCPAPE LLGGPSVFLF PPKPKDTLMI | 240 |
| SRTPEVTCVV VDVSHEDPEV KFNWYVDGVE VRNAKTKPRE EQYNSTYRVV SVLTVLHQDW | 300 |
| LNGKEYKCKV SNKALPAPIE KTISKAKGQP REPQVYTLPP SRDELTKNQV SLTCLVKGFY | 360 |
| PSDIAVEWES NGQPENNYKT TPPVLDSDGS FFLYSKLTVD KSRWQQGNVF SCSVMHEALH | 420 |
| NHYTQKSLSL SPGK | 434. |

A representative non-human primate (Cynomolgus) B7-DC fusion protein is encoded by a nucleic acid having at least 80%, 85%, 90%, 95%, 99% or 100% sequence identity to:

B7-1

A representative murine B7-1 fusion protein is encoded by a nucleic acid having at least 80%, 85%, 90%, 95%, 99% or 100% sequence identity to:

| (SEQ ID NO: 88) | |
|---|---|
| atggcttgca attgtcagtt gatgcaggat acaccactcc tcaagtttcc atgtccaagg | 60 |
| ctcattcttc tctttgtgct gctgattcgt ctttcacaag tgtcttcaga tgttgatgaa | 120 |
| caactgtcca agtcagtgaa agataaggta ttgctgcctt gccgttacaa ctctcctcat | 180 |
| gaagatgagt ctgaagaccg aatctactgg caaaaacatg acaaagtggt gctgtctgtc | 240 |
| attgctggga aactaaaagt gtggcccgag tataagaacc ggactttata tgacaacact | 300 |
| acctactctc ttatcatcct gggcctggtc ctttcagacc ggggcacata cagctgtgtc | 360 |
| gttcaaaaga aggaaagagg aacgtatgaa gttaaacact tggctttagt aaagttgtcc | 420 |
| atcaaagctg acttctctac ccccaacata actgagtctg gaaacccatc tgcagacact | 480 |
| aaaaggatta cctgctttgc ttccgggggt ttcccaaagc ctcgcttctc ttggttggaa | 540 |
| aatggaagag aattacctgg catcaatacg acaatttccc aggatcctga atctgaattg | 600 |
| tacaccatta gtagccaact agatttcaat acgactcgca accacaccat taagtgtctc | 660 |
| attaaatatg gagatgctca cgtgtcagag gacttcacct gggaaaaacc cccagaagac | 720 |
| cctcctgata gcaagaacga gccaagaggt cctacgatca agccctgccc gccttgtaaa | 780 |
| tgcccagctc caaatttgct gggtggaccg tcagtcttta tcttcccgcc aaagataaag | 840 |
| gacgtcttga tgattagtct gagccccatc gtgacatgcg ttgtggtgga tgtttcagag | 900 |
| gatgaccccg acgtgcaaat cagttggttc gttaacaacg tggaggtgca taccgctcaa | 960 |
| acccagaccc acagagagga ttataacagc accctgcggg tagtgtccgc cctgccgatc | 1020 |
| cagcatcagg attggatgag cgggaaagag ttcaagtgta aggtaaacaa caaagatctg | 1080 |
| ccagcgccga ttgaacgaac cattagcaag ccgaaaggga gcgtgcgcgc acctcaggtt | 1140 |
| tacgtccttc ctccaccaga agaggagatg acgaaaaagc aggtgaccct gacatgcatg | 1200 |
| gtaactgact ttatgccaga agatatttac gtggaatgga ctaataacgg aaagacagag | 1260 |
| ctcaattaca agaacactga gcctgttctg gattctgatg gcagctactt tatgtactcc | 1320 |
| aaattgaggg tcgagaagaa gaattgggtc gagagaaaca gttatagttg ctcagtggtg | 1380 |
| catgagggcc tccataatca tcacaccaca aagtccttca gccgaacgcc cgggaaa | 1437 |

The murine B7-1 fusion protein encoded by SEQ ID NO:88 has the following amino acid sequence:

| (SEQ ID NO: 89) | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLIILGLV LSDRGTYSCV | 120 |
| VQKKERGTYE VKHLALVKLS IKADFSTPNI TESGNPSADT KRITCFASGG FPKPRFSWLE | 180 |
| NGRELPGINT TISQDPESEL YTISSQLDFN TTRNHTIKCL IKYGDAHVSE DFTWEKPPED | 240 |
| PPDSKNEPRG PTIKPCPPCK CPAPNLLGGP SVFIFPPKIK DVLMISLSPI VTCVVVDVSE | 300 |
| DDPDVQISWF VNNVEVHTAQ TQTHREDYNS TLRVVSALPI QHQDWMSGKE FKCKVNNKDL | 360 |
| PAPIERTISK PKGSVRAPQV YVLPPPEEEM TKKQVTLTCM VTDFMPEDIY VEWTNNGKTE | 420 |
| LNYKNTEPVL DSDGSYFMYS KLRVEKKNWV ERNSYSCSVV HEGLHNHHTT KSFSRTPGK | 479 |

The amino acid sequence of the murine 137-1 fusion protein of SEQ ID NO:89 without the signal sequence is:

| (SEQ ID NO: 90) | |
|---|---|
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHLALV KLSIKADFST PNITESGNPS | 120 |
| ADTKRITCFA SGGFPKPRFS WLENGRELPG INTTISQDPE SELYTISSQL DFNTTRNHTI | 180 |
| KCLIKYGDAH VSEDFTWEKP PEDPPDSKNE PRGPTIKPCP PCKCPAPNLL GGPSVFIFPP | 240 |
| KIKDVLMISL SPIVTCVVVD VSEDDPDVQI SWFVNNVEVH TAQTQTHRED YNSTLRVVSA | 300 |
| LPIQHQDWMS GKEFKCKVNN KDLPAPIERT ISKPKGSVRA PQVYVLPPPE EEMTKKQVTL | 360 |
| TCMVTDFMPE DIYVEWTNNG KTELNYKNTE PVLDSDGSYF MYSKLRVEKK NWVERNSYSC | 120 |
| SVVHEGLHNH HTTKSFSRTP GK | 442. |

A representative human B7-1 fusion protein is encoded by a nucleic acid having at least 80%, 85%, 90%, 95%, 99% or 100% sequence identity to:

| (SEQ ID NO: 91) | |
|---|---|
| atgggccaca cacggaggca gggaacatca ccatccaagt gtccatacct caatttcttt | 60 |
| cagctcttgg tgctggctgg tctttctcac ttctgttcag gtgttatcca cgtgaccaag | 120 |
| gaagtgaaag aagtggcaac gctgtcctgt ggtcacaatg tttctgttga agagctggca | 180 |
| caaactcgca tctactggca aaaggagaag aaaatggtgc tgactatgat gtctggggac | 240 |
| atgaatatat ggcccgagta caagaaccgg accatctttg atatcactaa taacctctcc | 300 |
| attgtgatcc tggctctgcg cccatctgac gagggcacat acgagtgtgt tgttctgaag | 360 |
| tatgaaaaag acgctttcaa gcgggaacac ctggctgaag tgacgttatc agtcaaagct | 420 |
| gacttcccta cacctagtat atctgacttt gaaattccaa cttctaatat tagaaggata | 480 |
| atttgctcaa cctctggagg ttttccagag cctcacctct cctggttgga aaatggagaa | 540 |
| gaattaaatg ccatcaacac aacagtttcc caagatcctg aaactgagct ctatgctgtt | 600 |
| agcagcaaac tggatttcaa tatgacaacc aaccacagct tcatgtgtct catcaagtat | 660 |
| ggacatttaa gagtgaatca gaccttcaac tggaatacaa ccaagcaaga gcattttcct | 720 |
| gataacctgg agcctaagtc atgtgacaag acccatacgt gcccaccctg tcccgctcca | 780 |
| gaactgctgg ggggacctag cgttttcttg ttccccccaa agcccaagga caccctcatg | 840 |
| atctcacgga ctcccgaagt aacatgcgta gtagtcgacg tgagccacga ggatcctgaa | 900 |
| gtgaagttta attggtacgt ggacggagtc gaggtgcata atgccaaaac taaacctcgg | 960 |
| gaggagcagt ataacagtac ctaccgcgtg gtatccgtct tgacagtgct ccaccaggac | 1020 |
| tggctgaatg gtaaggagta taaatgcaag gtcagcaaca aagctcttcc cgccccaatt | 1080 |
| gaaaagacta tcagcaaggc caagggacaa ccccgcgagc cccaggttta cacccttcca | 1140 |
| ccttcacgag acgagctgac caagaaccag gtgtctctga cttgtctggt caaaggtttc | 1200 |
| tatccttccg acatcgcagt ggagtgggag tcaaacgggc agcctgagaa taactacaag | 1260 |
| accacacccc cagtgcttga tagcgatggg agctttttcc tctacagtaa gctgactgtg | 1320 |
| gacaaatccc gctggcagca gggaaacgtt ttctcttgta gcgtcatgca tgaggccctc | 1380 |
| cacaaccatt atactcagaa aagcctgagt ctgagtcccg gcaaa | 1425 |

The human B7-1 fusion protein encoded by SEQ ID NO:91 has the following amino acid sequence:

| (SEQ ID NO: 92) | |
|---|---|
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DEPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNLEPKSCDK THTCPPCPAP ELLGGPSVFL FPPKPKDTLM ISRTPEVTCV VVDVSHEDPE | 300 |
| VKFNWYVDGV EVHNAKTKPR EEQYNSTYRV VSVLTVLHQD WLNGKEYKCK VSNYALPAPI | 360 |
| EKTISKAKGQ PREPQVYTLP PSRDELTKNQ VSLTCLVKGF YPSDIAVEWE SNGQPENNYK | 420 |
| TTPPVLDSDG SFFLYSKLTV DKSRWQQGNV FSCSVMHEAL HNHYTQKSLS LSPGK | 475 |

The amino acid sequence of the human B7-1 fusion protein of SEQ ID NO:92 without the signal sequence is:

| (SEQ ID NO: 93) | |
|---|---|
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNLE PKSCDKTHTC PPCPAPELLG GPSVFLFPPK | 240 |
| PKDTLMISRT PEVTCVVVDV SHEDPEVKFN WYVDGVEVHN AKTKPREEQY NSTYRVVSVL | 300 |
| TVLHQDWLNG KEYKCKVSNK ALPAPIEKTI SKAKGQPREP QVYTLPPSRD ELTKNQVSLT | 360 |
| CLVKGFYPSD IAVEWESNGQ PENNYKTTPP VLDSDGSFFL YSKLTVDKSR WQQGNVFSCS | 420 |
| VMHEALHNHY TQKSLSLSPG K | 441. |

B7-2

A representative murine B7-2 fusion protein is encoded by a nucleic acid having at least 80%, 85%, 90%, 95%, 99% or 100% sequence identity to:

| (SEQ ID NO: 94) | |
|---|---|
| atggacccca gatgcaccat gggcttggca atccttatct ttgtgacagt cttgctgatc | 60 |
| tcagatgctg tttccgtgga gacgcaagct tatttcaatg ggactgcata tctgccgtgc | 120 |
| ccatttacaa aggctcaaaa cataagcctg agtgagctgg tagtattttg gcaggaccag | 180 |
| caaaagttgg ttctgtacga gcactatttg ggcacagaga aacttgatag tgtgaatgcc | 240 |
| aagtacctgg gccgcacgag ctttgacagg aacaactgga ctctacgact tcacaatgtt | 300 |
| cagatcaagg acatgggctc gtatgattgt tttatacaaa aaaagccacc cacaggatca | 360 |
| attatcctcc aacagacatt aacagaactg tcagtgatcg ccaacttcag tgaacctgaa | 420 |
| ataaaactgg ctcagaatgt aacaggaaat tctggcataa atttgacctg cacgtctaag | 480 |
| caaggtcacc cgaaacctaa gaagatgtat tttctgataa ctaattcaac taatgagtat | 540 |
| ggtgataaca tgcagatatc acaagataat gtcacagaac tgttcagtat ctccaacagc | 600 |
| ctctctcttt cattcccgga tggtgtgtgg catatgaccg ttgtgtgtgt tctggaaacg | 660 |
| gagtcaatga agatttcctc caaacctctc aatttcactc aagagtttcc atctcctcaa | 720 |
| acgtattgga aggagccaag aggtcctacg atcaagccct gcccgccttg taaatgccca | 780 |
| gctccaaatt tgctgggtgg accgtcagtc tttatcttcc cgccaaagat aaaggacgtc | 840 |
| ttgatgatta gtctgagccc catcgtgaca tgcgttgtgg tggatgtttc agaggatgac | 900 |
| cccgacgtgc aaatcagttg gttcgttaac aacgtggagg tgcataccgc tcaaacccag | 960 |
| acccacagag aggattataa cagcaccctg cgggtagtgt ccgccctgcc gatccagcat | 1020 |
| caggattgga tgagcgggaa agagttcaag tgtaaggtaa acaacaaaga tctgccagcg | 1080 |
| ccgattgaac gaaccattag caagccgaaa gggagcgtgc gcgcacctca ggtttacgtc | 1140 |
| cttcctccac cagaagagga gatgacgaaa aagcaggtga ccctgacatg catggtaact | 1200 |
| gactttatgc cagaagatat ttacgtggaa tggactaata acggaaagac agagctcaat | 1260 |
| tacaagaaca ctgagcctgt tctggattct gatggcagct actttatgta ctccaaattg | 1320 |
| agggtcgaga agaagaattg ggtcgagaga aacagttata gttgctcagt ggtgcatgag | 1380 |
| ggcctccata atcatcacac cacaaagtcc ttcagccgaa cgcccgggaa a | 1431 |

The murine B7-2 fusion protein encoded by SEQ ID NO:84 has the following amino acid sequence:

| (SEQ ID NO: 95) | |
|---|---|
| MDPRCTMGLA ILIFVTVLLI SDAVSVETQA YFNGTAYLPC PFTKAQNISL SELVVFWQDQ | 60 |
| QKLVLYEHYL GTEKLDSVNA KYLGRTSFDR NNWTLRLHNV QIKDMGSYDC FIQKKPPTGS | 120 |
| IILQQTLTEL SVIANFSEPE IKLAQNVTGN SGINLTCTSK QGHPKPKKMY FLITNSTNEY | 180 |
| GDNMQISQDN VTELFSISNS LSLSFPDGVW HMTVVCVLET ESMKISSKPL NFTQEFFSPQ | 240 |
| TYWKEPRGPT IKPCPPCKCP APNLLGGPSV FIFPPKIKDV LMISLSPIVT CVVVDVSEDD | 300 |
| PDVQISWFVN NVEVHTAQTQ THREDYNSTL RVVSALPIQH QDWMSGKEFK CKVNNXIMPA | 360 |
| PIERTISKPK GSVRAPQVYV LPPPEEEMTK KQVTLTCMVT DFMPEDIYVE WTNNGKTELN | 420 |
| YKNTEPVLDS DGSYFMYSKL RVEKKNWVER NSYSCSVVHE GLHNHHTTKS FSKTPGK | 477 |

The amino acid sequence of the murine B7-2 fusion protein of SEQ ID NO:95 without the signal sequence is:

| (SEQ ID NO: 96) | |
|---|---|
| VSVETQAYFN GTAYLPCPFT KAQN1SLSEL VVFWQDQQKL VLYEHYLGTE KLDSVNAKYL | 60 |
| GRTSFDRNNW TLRLHNVQIK DMGSYDCFIQ KKPPTGSIIL QQTLTELSVI ANFSEPEIKL | 120 |
| AQNVTGNSGI NLTCTSKQGH PKPKKMYFLI TNSTNEYGDN MQISQDNVTE LFSISNSLSL | 180 |
| SFPDGVNBMT VVCVLETESM KISSKPLNFT QEFFSPQTYW KEPRGPTIKP CPPCKCPAPN | 240 |
| LLGGPSVFIF PPKIKDVLMI SLSPIVTCVV VDVSEDDPDV QISWFVNNVE VHTAQTQTHR | 300 |
| EDYNSTLRVV SALPIQHQDW MSGKEFKCKV NNKDLPAPIE RTISKPKGSV RAPQVYVLPP | 360 |
| PEEEMTKKQV TLTCMVTDFM PEDIYVEWTN NGKTELNYKN TEPVLDSDGS YEMYSKLRVE | 420 |
| KKNWVERNSY SCSVVHEGLH NHHTTKSFSR TPGK | 454. |

A representative human B7-2 fusion protein is encoded by a nucleic acid having at least 80%, 85%, 90%, 95%, 99% or 100% sequence identity to:

| (SEQ ID NO: 97) | |
|---|---|
| atgggactga gtaacattct ctttgtgatg gccttcctgc tctctggtgc tgctcctctg | 60 |
| aagattcaag cttatttcaa tgagactgca gacctgccat gccaatttgc aaactctcaa | 120 |
| aaccaaagcc tgagtgagct agtagtattt tggcaggacc aggaaaactt ggttctgaat | 180 |
| gaggtatact taggcaaaga gaaatttgac agtgttcatt ccaagtatat gggccgcaca | 240 |
| agttttgatt cggacagttg gaccctgaga cttcacaatc ttcagatcaa ggacaagggc | 300 |
| ttgtatcaat gtatcatcca tcacaaaaag cccacaggaa tgattcgcat ccaccagatg | 360 |
| aattctgaac tgtcagtgct tgctaacttc agtcaacctg aaatagtacc aatttctaat | 420 |
| ataacagaaa atgtgtacat aaatttgacc tgctcatcta tacacggtta cccagaacct | 480 |
| aagaagatga gtgttttgct aagaaccaag aattcaacta tcgagtatga tggtgttatg | 540 |
| cagaaatctc aagataatgt cacagaactg tacgacgttt ccatcagctt gtctgtttca | 600 |
| ttccctgatg ttacgagcaa tatgaccatc ttctgtattc tggaaactga caagacgcgg | 660 |
| cttttatctt cacctttctc tatagagctt gaggaccctc agcctccccc agaccacatt | 720 |
| ccttggatta cagctgtact tgagcctaag tcatgtgaca agacccatac gtgcccaccc | 780 |
| tgtcccgctc cagaactgct ggggggacct agcgttttct tgttcccccc aaagcccaag | 840 |
| gacaccctca tgatctcacg gactcccgaa gtaacatgcg tagtagtcga cgtgagccac | 900 |
| gaggatcctg aagtgaagtt taattggtac gtggacggag tcgaggtgca taatgccaaa | 960 |
| actaaacctc gggaggagca gtataacagt acctaccgcg tggtatccgt cttgacagtg | 1020 |
| ctccaccagg actggctgaa tggtaaggag tataaatgca aggtcagcaa caaagctctt | 1080 |
| cccgccccaa ttgaaaagac tatcagcaag gccaagggac aaccccgcga gccccaggtt | 1140 |
| tacacccttc caccttcacg agacgagctg accaagaacc aggtgtctct gacttgtctg | 1200 |
| gtcaaaggtt tctatccttc cgacatcgca gtggagtggg agtcaaacgg gcagcctgag | 1260 |
| aataactaca agaccacacc cccagtgctt gatagcgatg ggagcttttt cctctacagt | 1320 |
| aagctgactg tggacaaatc ccgctggcag cagggaaacg ttttctcttg tagcgtcatg | 1380 |
| catgaggccc tccacaacca ttatactcag aaaagcctga gtctgagtcc cggcaaa | 1437 |

The human B7-2 fusion protein encoded by SEQ ID NO:97 has the following amino acid sequence:

| (SEQ ID NO: 98) | |
|---|---|
| MGLSNILFVM AFLLSGAAPL KIQAYFNETA DLPCQFANSQ NQSLSELVVF WQDQENLVLN | 60 |
| EVYLGKEKFD SVHSKYMGRT SFDSDSWTLR LHNLQIKDKG LYQCIIHHKK PTGMIRIHQM | 120 |
| NSELSVLANF SQPEIVPISN ITENVYINLT CSSIHGYPEP KKMSVLLRTK NSTIEYDGVM | 180 |
| QKSQDNVTEL YDVSISLSVS FPDVTSNMTI FCILETDKTR LLSSPFSIEL EDPQPPPDHI | 240 |
| PWITAVLEPK SCDKTHTCPP CPAPELLGGP SVFLFPPKPK DTLMISRTPE VTCVVVDVSH | 300 |
| EDPEVKFNWY VDGVEVHNAK TKPREEQYNS TYRVVSVLTV LHQDWLNGKE YKCKVSNKAL | 360 |
| PAPIEKTISK AKGQPREPQV YTLPPSRDEL TKNQVSLTCL VKGFYPSDIA VEWESNGOPE | 420 |
| NNYKTTPPVL DSDGSFFLYS KLTVDKSRWQ QGNVFSCSVM HEALHNHYTQ KSLSLSPGK | 479 |

The amino acid sequence of the human B7-2 fusion protein of SEQ ID NO:98 without the signal sequence is:

| (SEQ ID NO: 99) | |
|---|---|
| AYFNETADLP CQFANSQNQS LSELVVFWQD QENLVLNEVY LGKEKFDSVH SKYMGRTSFD | 60 |
| SDSWTLRLHN LQIKDKGLYQ CIIHHKKPTG MIRIHQMNSE LSVLANFSQP EIVPISNITE | 120 |
| NVYINLTCSS IHGYPEPKKM SVLLRTKNST IEYDGVMQKS QDNVTELYDV SISLSVSFPD | 180 |
| VTSNMTIFCI LETDKTRLLS SPFSIELEDP QPPPDHIPWI TAVLEPKSCD KTHTCPPCPA | 240 |
| PELLGGPSVF LFPPKPKDTL MISRTPEVTC VVVDVSHEDP EVKFNWYVDG VEVHNAKTKP | 300 |
| REEQYNSTYR VVSVLTVLHQ DWLNGKEYKC KVSNKALPAP IEKTISKAKG QPREPQVYTL | 360 |
| PPSRDELTKN QVSLTCLVKG FYPSDIAVEW ESNGQPENNY KTTPPVLDSD GSFFLYSKLT | 420 |
| VDKSRWQQGN VFSCSVMHEA LHNHYTQKSL SLSPGK | 456 |

B7-H5

A representative murine B7-H5 fusion protein is encoded by a nucleic acid having at least 80%, 85%, 90%, 95%, 99% or 100% sequence identity to:

| (SEQ ID NO: 100) | |
|---|---|
| atgggtgtcc ccgcggtccc agaggccagc agcccgcgct ggggaaccct gctccttgct | 60 |
| attttcctgg ctgcatccag aggtctggta gcagccttca aggtcaccac tccatattct | 120 |
| ctctatgtgt gtcccgaggg acagaatgcc accctcacct gcaggattct gggccccgtg | 180 |
| tccaaagggc acgatgtgac catctacaag acgtggtacc tcagctcacg aggcgaggtc | 240 |
| cagatgtgca aagaacaccg gcccatacgc aacttcacat tgcagcacct tcagcaccac | 300 |
| ggaagccacc tgaaagccaa cgccagccat gaccagcccc agaagcatgg gctagagcta | 360 |
| gcttctgacc accacggtaa cttctctatc accctgcgca atgtgacccc aagggacagc | 420 |
| ggcctctact gctgtctagt gatagaatta aaaaaccacc acccagaaca acggttctac | 480 |
| gggtccatgg agctacaggt acaggcaggc aaaggctcgg ggtccacatg catggcgtct | 540 |
| aatgagcagg acagtgacag catcacggct gagccaagag gtcctacgat caagccctgc | 600 |
| ccgccttgta aatgcccagc tccaaatttg ctgggtggac cgtcagtctt tatcttcccg | 660 |
| ccaaagataa aggacgtctt gatgattagt ctgagcccca tcgtgacatg cgttgtggtg | 720 |
| gatgtttcag aggatgaccc cgacgtgcaa atcagttggt tcgttaacaa cgtggaggtg | 780 |
| cataccgctc aaacccagac ccacagagag gattataaca gcaccctgcg ggtagtgtcc | 840 |
| gccctgccga tccagcatca ggattggatg agcgggaaag agttcaagtg taaggtaaac | 900 |
| aacaaagatc tgccagcgcc gattgaacga accattagca agccgaaagg gagcgtgcgc | 960 |
| gcacctcagg tttacgtcct tcctccacca gaagaggaga tgacgaaaaa gcaggtgacc | 1020 |
| ctgacatgca tggtaactga ctttatgcca gaagatattt acgtggaatg gactaataac | 1080 |
| ggaaagacag agctcaatta caagaacact gagcctgttc tggattctga tggcagctag | 1140 |
| tttatgtact ccaaattgag ggtcgagaag aagaattggg tcgagagaaa cagttatagt | 1200 |
| tgctcagtgg tgcatgaggg cctccataat catcacacca caaagtcctt cagccgaacg | 1260 |
| cccgggaaa | 1269 |

The murine B7-H5 fusion protein encoded by SEQ ID NO:100 has the following amino acid sequence:

| (SEQ ID NO: 101) | |
|---|---|
| MGVPAYPEAS SPRWGTLLLA IFLAASRGLV AAFKVTTPYS LYVCPEGQNA TLTCRILGPV | 60 |
| SKGHDVTIYK TWYLSSRGEV QMCKEHRPIR NFTLQHLQHH GSHLKANASH DQPQKHGLEL | 120 |
| ASDHHGNFSI TLRNVTPRDS GLYCCLVIEL KNHHPEQRFY GSMELQVQAG KGSGSTCMAS | 160 |
| NEQDSDSITA EPRGPTIKPC PPCKCPAPNL LGGPSVFIFP PKIKDVLMIS LSPIVTCVVV | 240 |
| DVSEDDPDVQ ISWFVNNVEV HTAQTQTHRE DYNSTLRVVS ALPIQHQDWM SGKEFKCKVN | 300 |
| NKDLPAPIER TISKPKGSVR APQVYVLPPP EEEMTKKQVT LTCMVTDFMP EDIYVEWTNN | 360 |
| GKTELNYKNT EPVLDSDGSY FMYSKLRVEK KNWVERNSYS CSVVHEGLHN HHTTKSFSRT | 420 |
| PGK | 423 |

The amino acid sequence of the murine B7-H5 fusion protein of SEQ ID NO:101 without the signal sequence is:

| (SEQ ID NO: 102) | |
|---|---|
| FKVTTPYSLY VCPEGQNATL TCRILGPVSK GHDVTIYKTW YLSSRGEVQM CKEHRPIRNF | 60 |
| TLQHLQHHGS HLKANASHDQ PQKHGLELAS DHHGNFSITL RNVTPRDSGL YCCLVIELKN | 120 |
| HHPEQRFYGS MELQVQAGKG SGSTCMASNE QDSDSITAEP RGPTIKPCPP CKCPAPNLLG | 180 |
| GPSVFIFETK IKDVLMISLS PIVTCVVVDV SEDDPDVQIS WFVNNVEVHT AQTQTHREDY | 240 |
| NSTLRVVSAL PIQHQDWMSG KEFKCKVNNK DLPAPIERTI SKPKGSVRAP QVYVLPPPEE | 300 |
| EMTKKQVTLT CMVTDFMPED IYVEWTNNGK TELNYKNTEP VLDSDGSYFM YSKLRVEKKN | 360 |
| WVERNSYSCS VVHEGLHNHH TTKSFSRTPG K | 391. |

A representative human B7-H5 fusion protein is encoded by a nucleic acid having at least 80%, 85%, 90%, 95%, 99% or 100% sequence identity to:

| (SEQ ID NO: 103) | |
|---|---|
| atgggcgtcc ccacggccct ggaggccggc agctggcgct ggggatccct gctcttcgct | 60 |
| ctcttcctgg ctgcgtccct aggtccggtg gcagccttca aggtcgccac gccgtattcc | 120 |
| ctgtatgtct gtcccgaggg gcagaacgtc accctcacct gcaggctctt gggccctgtg | 180 |
| gacaaagggc acgatgtgac cttctacaag acgtggtacc gcagctcgag gggcgaggtg | 240 |
| cagacctgct cagagcgccg gcccatccgc aacctcacgt tccaggacct tcacctgcac | 300 |
| catggaggcc accaggctgc caacaccagc cacgacctgg ctcagcgcca cgggctggag | 360 |
| tcggcctccg accaccatgg caacttctcc atcaccatgc gcaacctgac cctgctggat | 420 |
| agcggcctct actgctgcct ggtggtggag atcaggcacc accactcgga gcacagggtc | 480 |
| catggtgcca tggagctgca ggtgcagaca ggcaaagatg caccatccaa ctgtgtggtg | 540 |
| tacccatcct cctcccagga tagtgaaaac atcacggctg agcctaagtc atgtgacaag | 600 |
| acccatacgt gcccaccctg tcccgctcca gaactgctgg ggggacctag cgttttcttg | 660 |
| ttccccccaa agcccaagga caccctcatg atctcacgga ctcccgaagt aacatgcgta | 720 |
| gtagtcgacg tgagccacga ggatcctgaa gtgaagttta attggtacgt ggacggagtc | 780 |
| gaggtgcata atgccaaaac taaacctcgg gaggagcagt ataacagtac ctaccgcgtg | 840 |
| gtatccgtct tgacagtgct ccaccaggac tggctgaatg gtaaggagta taaatgcaag | 900 |
| gtcagcaaca aagctcttcc cgccccaatt gaaaagacta tcagcaaggc caagggacaa | 960 |
| ccccgcgagc cccaggttta cacccttcca ccttcacgag acgagctgac caagaaccag | 1020 |
| gtgtctctga cttgtctggt caaaggtttc tatccttccg acatcgcagt ggagtgggag | 1080 |
| tcaaacgggc agcctgagaa taactacaag accacacccc cagtgcttga tagcgatggg | 1140 |
| agctttttcc tctacagtaa gctgactgtg gacaaatccc gctggcagca gggaaacgtt | 1200 |
| ttctcttgta gcgtcatgca tgaggccctc cacaaccatt atactcagaa aagcctgagt | 1260 |
| ctgagtcccg gcaaa | 1275 |

The human B7-H5 fusion protein encoded by SEQ ID NO:103 has the following amino acid sequence:

| (SEQ ID NO: 104) | |
|---|---|
| MGVPTALEAG SWRWGSLLFA LFLARSLGPV AAFKVATPYS LYVCPEGQNV TLTCRLLGPV | 60 |
| DKGHDVTFYK TWYRSSRGEV QTCSERRPIR NLTFQDLHLH HGGHQAANTS HDLAQRHGLE | 120 |
| SASDHHGNFS ITMRNLTLLD SGLYCCLVVE IRHHHSEHRV HGAMELQVQT GKDAPSNCVV | 180 |
| YPSSSQDSEN ITAEPKSCDK THTCPPCPAP ELLGGPSVFL FPPKPKDTLM ISRTPEVTCV | 240 |
| VVDVSHEDPE VKFNWYVDGV EVHNAKTKPR EEQYNSTYRV VSVLTVLAQD WLNGKEYKCK | 300 |
| VSNKALPAPI EKTISKAKGQ PREPQVYTLP PSRDELTKNQ VSLTCLVKGF YPSDIAVEWE | 360 |
| SNGQPENNYK TTPPVLDSDG SFFLYSKLTV DKSRWQQGNV FSCSVMHEAL HNHYTQKSLS | 420 |
| LSPGK | 425 |

The amino acid sequence of the human B7-H5 fusion protein of SEQ ID NO:104 without the signal sequence is:

| (SEQ ID NO: 105) | |
|---|---|
| FKVATPYSLY VCPEGQNVTL TCRLLGPVDK GHDVTFYKTW YRSSRGEVQT CSERRPIRNL | 60 |
| TFQDLHLHHG GHQAANTSHD LAQRHGLESA SDHHGNFSIT MRNLTLLDSG LYCCLVVEIR | 120 |
| HHHSEHRVHG AMELQVQTGK DAPSNCVVYP SSSQDSENIT AEPKSCDKTH TCPPCPAPEL | 180 |
| LGGPSVFLFP PKPKDTLMIS RTPEVTCVVV DVSHEDPEVK FNWYVDGVEV HNAKTKPREE | 240 |
| QYNSTYRVVS VLTVLHQDWL NGKEYKCKVS NKALPAPIEK TISKAKGQPR EPQVYTLPPS | 300 |
| RDELTKNQVS LTCLVKGFYP SDIAVEWESN GQPENNYKTT PPVLDSDGSF FLYSKLTVDK | 360 |
| SRWQQGNVES CSVMHEALHN HYTQKSLSLS PGK | 393. |

F. Fusion Protein Dimers and Multimers

The fusion proteins disclosed herein can be dimerized or multimerized. Dimerization or multimerization can occur between or among two or more fusion proteins through dimerization or multimerization domains, including those described above. Alternatively, dimerization or multimerization of fusion proteins can occur by chemical crosslinking. Fusion protein dimers can be homodimers or heterodimers. Fusion protein multimers can be homomultimers or heteromultimers.

Fusion protein dimers as disclosed herein are of formula II:

N—R₁—R₂—R₃—C

N—R₄—R₅—R₆—C

or, alternatively, are of formula III:

N—R₁—R₂—R₃—C

C—R₄—R₅—R₆—N

wherein the fusion proteins of the dimer provided by formula II are defined as being in a parallel orientation and the fusion proteins of the dimer provided by formula III are defined as being in an antiparallel orientation. Parallel and antiparallel dimers are also referred to as cis and trans dimers, respectively. “N” and “C” represent the N- and C-termini of the fusion protein, respectively. The fusion protein constituents “R₁”, “R₂” and “R₃” are as defined above with respect to formula I. With respect to both formula II and formula III, “R₄” is a costimulatory polypeptide domain or a antigen-binding targeting domain, “R₅” is a peptide/polypeptide linker domain, and “R₆” is a costimulatory polypeptide domain or a antigen-binding targeting domain, wherein “R₆” is a costimulatory polypeptidedomain when “R₄” is a antigen-binding targeting domain, and “R₆” is a antigen-binding targeting domain when “R₄” is a costimulatory polypeptide domain. In one embodiment, when “R₁” is a costimulatory polypeptide domain, “R₄” is also a costimulatory polypeptidedomain, and “R₃” and “R₆” are both antigen-binding targeting domains. In another embodiment, when “R₁” is a antigen-binding targeting domains, “R₄” is also a antigen-binding targeting domains, and “R₃” and “R₆” are both costimulatory polypeptide domains. In a preferred embodiment, “R₁” and “R₄” are costimulatory polypeptide domains, and “R₃” and “R₆” are antigen-binding targeting domains.

Fusion protein dimers of formula II are defined as homodimers when “R₁”═“R₄”, “R₂”═“R₅” and “R₃”═“R₆”. Similarly, fusion protein dimers of formula III are defined as homodimers when “R₁”═“R₆”, “R₂”═“R₅” and “R₃”═“R₄”. Fusion protein dimers are defined as heterodimers when these conditions are not met for any reason. For example, heterodimers may contain domain orientations that meet these conditions (i.e., for a dimer according to formula II, “R₁” and “R₄” are both costimulatory polypeptide domains, “R₂” and “R₅” are both peptide/polypeptide liker domains and “R₃” and “R₆” are both antigen-binding targeting domains), however the species of one or more of these domains is not identical. For example, although “R₃” and “R₆” may both be antigen-binding targeting domains, they may each target a distinct antigen. Alternatively, “R₃” and “R₆” may both be antigen-binding targeting domains that target the same antigen, but may be distinct classes of binding domains (i.e., “R₃” is a natural ligand for a receptor and “R₆” is a single chain variable fragment (scFv) that binds to the same receptor).

Dimers of fusion proteins that contain either a C_{H}1 or C_{L} region of an immunoglobulin as part of the polypeptide linker domain preferably form heterodimers wherein one fusion protein of the dimer contains a C_{H}1 region and the other fusion protein of the dimer contains a C_{L} region.

Fusion proteins can also be used to form multimers. As with dimers, multimers may be parallel multimers, in which all fusion proteins of the multimer are aligned in the same orientation with respect to their N- and C-termini. Multimers may be antiparallel multimers, in which the fusion proteins of the multimer are alternatively aligned in opposite orientations with respect to their N- and C-termini. Multimers (parallel or antiparallel) can be either homomultimers or heteromultimers.

G. Peptide and Polypeptide Modifications

The disclosed fusion proteins may be modified by chemical moieties that may be present in polypeptides in a normal cellular environment, for example, phosphorylation, methylation, amidation, sulfation, acylation, glycosylation, sumoylation and ubiquitylation. Fusion proteins may also be modified with a label capable of providing a detectable signal, either directly or indirectly, including, but not limited to, radioisotopes and fluorescent compounds.

The fusion proteins disclosed herein may also be modified by chemical moieties that are not normally added to polypeptides in a cellular environment. Such modifications may be introduced into the molecule by reacting targeted amino acid residues of the polypeptide with an organic derivatizing agent that is capable of reacting with selected side chains or terminal residues. Another modification is cyclization of the protein.

Examples of chemical derivatives of the polypeptides include lysinyl and amino terminal residues derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Carboxyl side groups, aspartyl or glutamyl, may be selectively modified by reaction with carbodiimides (R—N═C═N—R′) such as 1-cyclohexyl-3-(2-morpholinyl-(4-ethyl)carbodiimide or 1-ethyl-3-(4-azonia-4,4-dimethylpentyl)carbodiimide. Furthermore, aspartyl and glutamyl residues can be converted to asparaginyl and glutaminyl residues by reaction with ammonia. Fusion proteins may also include one or more D-amino acids that are substituted for one or more L-amino acids.

### III. Isolated Nucleic Acid Molecules

Isolated nucleic acid sequences encoding the fusion proteins disclosed herein are also provided. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids encoding fusion polypeptides may be optimized for expression in the expression host of choice. Codons may be substituted with alternative codons encoding the same amino acid to account for differences in codon usage between the mammal from which the nucleic acid sequence is derived and the expression host. In this manner, the nucleic acids may be synthesized using expression host-preferred codons.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone.

Nucleic acids encoding polypeptides disclosed herein can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

One approach includes nucleic acid transfer into primary cells in culture followed by autologous transplantation of the ex vivo transformed cells into the host, either systemically or into a particular organ or tissue. In one embodiment, vectors containing nucleic acids encoding fusion proteins are transfected into cells that are administered to a subject in need thereof.

Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex viva gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject.

In vivo nucleic acid therapy can be accomplished by direct transfer of a functionally active DNA into mammalian somatic tissue or organ in viva. For example, nucleic acids encoding polypeptides disclosed herein can be administered directly to lymphoid tissues or tumors. Alternatively, lymphoid tissue specific targeting can be achieved using lymphoid tissue-specific transcriptional regulatory elements (TREs) such as a B lymphocyte-, T lymphocyte-, or dendritic cell-specific TRE. Lymphoid tissue specific TREs are known in the art.

Nucleic acids may also be administered in vivo by viral means. Nucleic acid molecules encoding fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

C. Vectors and Host Cells

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

Vectors containing mucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAF-dextran-mediated transfection, lipofection, electroporation, or microinjection. Host cells (e.g., a prokaryotic cell or a eukaryotic cell such as a CHO cell) can be used to, for example, produce the fusion proteins described herein. In some embodiments, a host cell (e.g., an antigen presenting cell) can be used to express the fusion proteins disclosed herein for presentation to a T cell.

### IV. Immunogenic Compositions

Vaccines require strong T cell response to eliminate cancer cells and infected cells. The fusion proteins described herein can be administered as a component of a vaccine to provide a costimulatory signal to T cells. Vaccines disclosed herein include antigens, a source of fusion proteins, and optionally, adjuvants.

A. Antigens

Antigens can be any substance that evokes an immunological response in a subject. Representative antigens include peptides, proteins, polysaccharides, saccharides, lipids, nucleic acids, or combinations thereof. The antigen can be derived from a tumor or from a transformed cell such as a cancer or leukemic cell and can be a whole cell or immunogenic component thereof, e.g., cell wall components or molecular components thereof.

Suitable antigens are known in the art and are available from commercial sources. The antigens may be purified or partially purified polypeptides derived from tumors or other sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

B. Fusion Proteins

Any of the fusion proteins disclosed herein are suitable for use in the immunogenic compositions. Sources of fusion proteins include any fusion protein or nucleic acid encoding any fusion protein disclosed herein, or host cells containing vectors that express any of the fusion proteins disclosed herein. The fusion proteins may be monomeric, homodimeric, heterodimeric, homomultimeric or heteromultimeric.

C. Adjuvants

Optionally, the vaccines described herein may include adjuvants. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Additional adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor. In addition to the fusion proteins disclosed herein, other costimulatory molecules, including other polypeptides of the B7 family, may be co-administered. Such proteinaceous adjuvants may be provided as the full-length polypeptide or an active fragment thereof, or in the form of DNA, such as plasmid DNA.

### V. Pharmaceutical Compositions

Pharmaceutical compositions including fusion polypeptides disclosed herein are provided. Pharmaceutical compositions containing peptides or polypeptides may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected. Therapeutically effective amounts of the fusion proteins disclosed herein cause an immune response against a tumor or an infectious agent to be activated or sustained. Therapeutically effective amounts of the fusion proteins disclosed herein also costimulate the subject's T cells.

For the compositions disclosed herein and nucleic acids encoding the same, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally dosage levels of 0.001 to 10 mg/kg of body weight daily are administered to mammals. Generally, for intravenous injection or infusion, dosage may be lower.

1. Formulations for Parenteral Administration

In a preferred embodiment, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Topical Administration

Fusion proteins disclosed herein can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

3. Controlled Delivery Polymeric Matrices

Fusion proteins disclosed herein may also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of fusion polypeptides or nucleic acids encoding the fusion polypeptides, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### VI. Methods of Manufacture

A. Methods for Producing Fusion Proteins

Isolated fusion proteins can be obtained by, for example, chemical synthesis or by recombinant production in a host cell. To recombinantly produce a fusion protein, a nucleic acid containing a nucleotide sequence encoding the fusion protein can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the fusion protein. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

Mammalian cell lines that stably express variant fusion proteins can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a fusion protein can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Fusion proteins can be isolated using, for example, chromatographic methods such as DEAE ion exchange, gel filtration, and hydroxylapatite chromatography. For example, a costimulatory polypeptide in a cell culture supernatant or a cytoplasmic extract can be isolated using a protein G column. In some embodiments, fusion proteins can be engineered to contain an additional domain containing amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides. Fusion proteins can additionally be engineered to contain a secretory signal (if there is not a secretory signal already present) that causes the fusion protein to be secreted by the cells in which it is produced. The secreted fusion proteins can then conveniently be isolated from the cell media.

B. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Fusion protein-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### VII. Methods of Use

A. Activation of T Cells

The fusion proteins disclosed herein, nucleic acids encoding the fusion proteins, or cells expressing the fusion proteins can be used to activate T cells (i.e., increase antigen-specific proliferation of T cells, enhance cytokine production by T cells, stimulate differentiation and effector functions of T cells and/or promote T cell survival).

Methods for using fusion proteins to activate T cell responses are disclosed herein. The methods include contacting a T cell with any of the molecules disclosed herein. Fusion proteins are a preferred example. An isolated fusion protein or a dimer or multimer of fusion proteins. The fusion protein or fusion protein dimer or multimer can be any of those described herein, including any of the disclosed amino acid alterations, polypeptide fragments, and combinations thereof.

With respect to variant costimulatory polypeptides used in the fusion proteins, the variants described herein can have reduced or increased binding to coinhibitory receptors (i.e. PD-1) relative to wild type costimulatrory polypeptides, yet retain the ability to costimulate T cells. Preferred variant costimulatory polypeptides have a enhanced ability to stimulate signaling through and activating receptor compared to a non-variant costimulatory polypeptide.

The contacting can be in vitro, ex vivo, or in vivo (e.g., in a mammal such as a mouse, rat, rabbit, dog, cow, pig, non-human primate, or a human). In a preferred embodiment, fusion proteins are administered to contact T cells in vivo. The contacting can occur before, during, or after activation of the T cell. In one embodiment, contacting of the T cell with fusion protein can be at substantially the same time as activation. Activation can be, for example, by exposing the T cell to an antibody that binds to the T cell receptor (TCR) or one of the polypeptides of the CD3 complex that is physically associated with the TCR. Alternatively, a T cell can be exposed to either an alloantigen (e.g., a MHC alloantigen) on, for example, an APC [e.g., an interdigitating dendritic cell (referred to herein as a dendritic cell), a macrophage, a monocyte, or a B cell] or an antigenic peptide produced by processing of a protein antigen by any of the above APC and presented to the T cell by MHC molecules on the surface of the APC. The T cell can be a CD4⁺ T cell or a CD8⁺ T cell.

If the activation is in vitro, the fusion proteins can be bound to the floor of a relevant culture vessel, e.g. a well of a plastic microtiter plate. In vitro application of the isolated variant costimulatory polypeptides can be useful, for example, in basic scientific studies of immune mechanisms or for production of activated T cells for use in studies of T cell function or, for example, passive immunotherapy. Furthermore, fusion proteins disclosed herein can be added to in vitro assays (e.g., T cell proliferation assays) designed to test for immunity to an antigen of interest in a subject from which the T cells were obtained. Addition of fusion proteins to such assays would be expected to result in a more potent, and therefore more readily detectable, in vitro response. Moreover, a fusion proteins disclosed herein or nucleic acids encoding them, can be used: (a) as a positive control in an assay to test for costimulatory activity in other molecules; or (b) in screening assays for compounds useful in inhibiting T costimulation (e.g., compounds potentially useful for treating autoimmune diseases or organ graft rejection).

B. Therapeutic Uses of Fusion Proteins

1. Activation of T Cell-Mediated Immune Responses to Cancer

The fusion proteins provided herein are generally useful in vivo and ex vivo as immune response-stimulating therapeutics. The fusion proteins are particularly useful in vivo for the induction of tumor immunity and immunity to agents that cause infectious diseases.

In some embodiments, the fusion proteins disclosed herein contain a domain that binds to an antigen, ligand, or receptor on tumors or tumor-associated neovasculature in the local tumor environment. The tumor or tumor-associated neovasculature binding domain functions to effectively target the fusion proteins to the local tumor microenvironment, where they can specifically enhance the activity of tumor-infiltrating effector T cells.

In other embodiments, the fusion proteins disclosed herein contain a domain that binds to an antigen, ligand or receptor on cells in tissues involved in regulating immune cell activation in response to infectious disease causing agents. Targeting the fusion proteins to tissues involved in immune cell activation allows for efficient activation of T cells and can cause local activation of T cell, resulting in long term immunity.

The ability of the fusion proteins to concentrate in tumors or immune tissues involved in immune cell activation also reduces the amount of costimulatory molecule that is necessary to administer in vivo to achieve therapeutic efficacy. The ability of the fusion proteins to concentrate in tumors or immune tissues involved in immune cell activation and the resulting reduction in the amount of costimulatory molecule that is necessary to administer in vivo to achieve therapeutic efficacy also reduces non-specific activation of the immune system. Non-specific activation of the immune system refers to activation of T cells or other immune cells that do not specifically recognize antigens expressed by a tumor or an infectious disease causing agent to be treated or are not involved directly or indirectly in the anti-tumor or anti-infection response. Non-specific activation of the immune response can lead to the development of inflammatory disorders and autoimmunity.

Fusion proteins can be administered as monomers or as dimers or multimers. Dimers and multimers can be homodimers/homomultimers or heterodimers/heteromultimers as described above. In a preferred embodiment, fusion proteins are administered as dimers or multimers. Administration of fusion proteins as dimers or multimers increases the valency of the fusion proteins. The increase in valency can result in an increase in the avidity of the fusion protein for its target antigen(s), receptor(s) or ligand(s) on the tumor, tumor-associated neovasculature, or tissue involved in immune cell activation, and thereby increase its retention in the tumor microenvironment or in the immune-regulating tissue. Increasing the valency of the fusion proteins can also increase their ability to cross-link costimulatory receptors on T cells.

1. Induction of Tumor Immunity

Some cancer patients have tumor-infiltrating, antigen specific cytotoxic T lymphocytes (TIL) that are able to kill tumor cells and reduce tumor burden. However, the frequency of patients with such responses and the number of TILs within the tumor is extremely low. Consequently, they are unable to eradicate the tumors. Human clinical trials in melanoma patients demonstrated that when these patients were treated with passive administration of high doses of antigen specific TIL expanded ex vivo, a significant number of tumors, including large tumors, were eradicated (Dudley, Science, 298:850-4 (2002)).

Compositions that are targeted to tumors or tumor-associated neovasculature and contain molecules that enhance the function of tumor-infiltrating T cells are provided herein. In certain embodiments it is believed that the compositions increase or augment the functional immune response against a tumor relative to a control by costimulating T cells or by inhibiting or reducing inhibitory signals to T cells in a subject. In a preferred embodiment, the compositions are formulated to increase the number or functional activity of tumor-infiltrating, antigen specific cytotoxic T lymphocytes (TILs) in a subject in need thereof.

One embodiment provides a method for increasing the activation of tumor-infiltrating leukocytes in a subject by administering to the subject an effective amount of a fusion protein disclosed herein or a nucleic acid encoding the same to activate the subject's T cells and/or to inhibit or reduce coinhibition of the subject's T cells.

Another embodiment provides a method for increasing the population of tumor-infiltrating leukocytes in a subject by administering to the subject an effective amount of a fusion protein disclosed herein or a nucleic acid encoding the same to costimulate the subject's T cells and/or to inhibit or reduce coinhibition of the subject's T cells.

Another embodiment provides a method for stimulating or augmenting an effective anti-tumor T cell response by administering to the subject an effective amount of a fusion protein disclosed herein or a nucleic acid encoding the same to activate the subject's T cells and/or to inhibit or block inhibition of the subject's T cells.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated in with the provided compositions and methods include, but are not limited to, the following: bladder, brain, breast, cervical, colo-rectal, esophageal, kidney, liver, lung, nasopharangeal, pancreatic, prostate, skin, stomach and uterine. Administration is not limited to the treatment of an existing tumor or infectious disease but can also be used to prevent or lower the risk of developing such diseases in an individual, i.e., for prophylactic use. Potential candidates for prophylactic vaccination include individuals with a high risk of developing cancer, i.e., with a personal or familial history of certain types of cancer.

2. Use of Fusion Proteins in Vaccines

The fusion proteins disclosed herein, and/or nucleic acids encoding the same may be administered alone or in combination with any other suitable treatment. In one embodiment, fusion proteins, and/or nucleic acids encoding the same may be administered in conjunction with, or as a component of, a vaccine composition. Suitable components of vaccine compositions are described above. Fusion protein compositions described herein can be administered prior to, concurrently with, or after the administration of a vaccine. In one embodiment the fusion protein composition is administered at the same time as administration of a vaccine.

The fusion proteins described herein may be administered in conjunction with prophylactic vaccines, which confer resistance in a subject to development of certain types of tumors, or in conjunction with therapeutic vaccines, which can be used to initiate or enhance a subject's immune response to a pre-existing antigen, such as a tumor antigen in a subject already having cancer.

The desired outcome of a prophylactic or therapeutic immune response may vary according to the disease, according to principles well known in the art. For example, an immune response against cancer, may completely treat the cancer or infectious disease, may alleviate symptoms, or may be one facet in an overall therapeutic intervention against the cancer or infectious disease.

C. Combination Therapy

The disclosed fusion protein compositions can be administered alone or in combination with one or more additional therapeutic agents. For example, the stimulation of an immune response against a cancer may be coupled with surgical, chemotherapeutic, radiologic, hormonal and other immunologic approaches in order to affect treatment.

For example, the disclosed fusion proteins can be administered with an antibody or antigen binding fragment thereof specific for growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II RIM-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor eceptor 4; nerve growth factor receptor (NGFR) (TrkA); BDNF receptor (TrkB); NT-3-receptor (TrkC); vascular endothelial growth factor receptor 1 (Flip; vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Ax1; RYK; DDR; and Tie.

Additional therapeutic agents include conventional cancer therapeutics such as chemotherapeutic agents, cytokines, chemokines, and radiation therapy. The majority of chemotherapeutic drugs can be divided into: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

### EXAMPLES

### Example 1

### P815 Mastocytoma Model

The in vivo activity of murine B7-DC-Ig was tested in the P815 mastocytoma tumor model. P815 mastocytoma cells were derived from DBA/2 mice after methylcholanthrene (MCA) treatment. Injection of 5×10⁴ cells SC can result in mortality approximately 35 days post tumor inoculation.

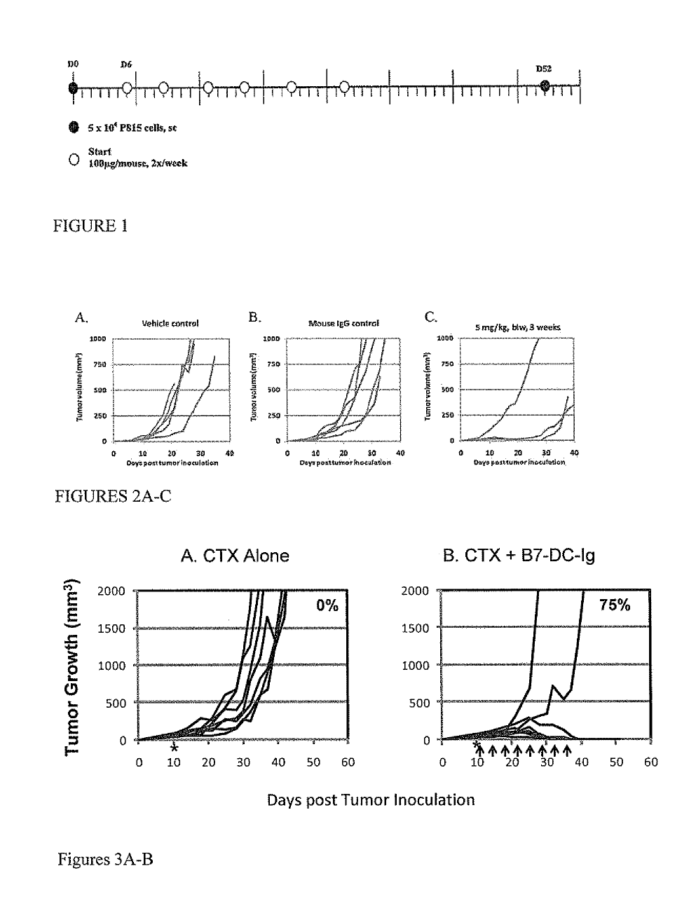

DBA/2 mice (6-10 weeks of age, females) were first challenged with 5×10⁴ live P815 cells injected SC in the flank. Six days later, the mice were treated with murine B7-DC-Ig via IP injection. The dosing regimen, shown in FIG. 1, was 100 μg of murine B7-DC-Ig per injection (approximately 5 mg/kg), 2 times per week, up to 6 doses. Control groups were treated with vehicle only or with murine IgG. Tumor size was measured with digital calipers every 2-3 days. Mice were euthanized and defined as dead when their tumor size reached or exceeded 1000 mm³, according to protocols approved by the Institutional Animal Care and Use Committee (IACUC) of the American Red Cross (ARC; the site of Amplimmune's vivarium). Surviving tumor free mice were re-challenged with P815 tumor cells on Day 52.

As shown in Table 1 and FIG. 1 Error! Reference source not found., all of the mice treated with vehicle or control mouse IgG required euthanasia by Day 38 because their tumor volumes reached the IACUC limit. Four of 5 murine B7-DC-Ig treated mice responded to treatment: tumor was eradicated in two mice and two additional mice showed delayed tumor growth during murine B7-DC-Ig treatment.

TABLE 1
P815 tumor model results.

| | | # Tumor | # Tumor | # Tumor |
|---|---|---|---|---|
| Group | Treatment | free | <500 mm³ | ≧500 mm³ |
| A | Vehicle control | 0 | 0 | 5 |
| B | Mouse IgG control | 0 | 0 | 5 |
| C | Murine B7-DC-Ig | 2 | 2 | 1 |
| | (5 mg/kg IP biw 3 weeks | | | |
| | starting Day 6) | | | |

FIGS. 2A-C show tumor eradication in mice using murine B7-DC-Ig. The tumor-free mice were then re-challenged with 5×10⁴P815 cells administered to the flank opposite the primary inoculation site on Day 52. The mice remained tumor free through 74 days after the primary inoculation, while all naïve mice challenged with P815 cells developed tumors. This suggests that mice inoculated with P815 cells and treated with murine B7-DC-Ig developed long-term immunity against P815 mastocytoma.

### Example 2

### Combination of Cyclophosphamide and B7-DC-Ig can Eradicate Established Tumors

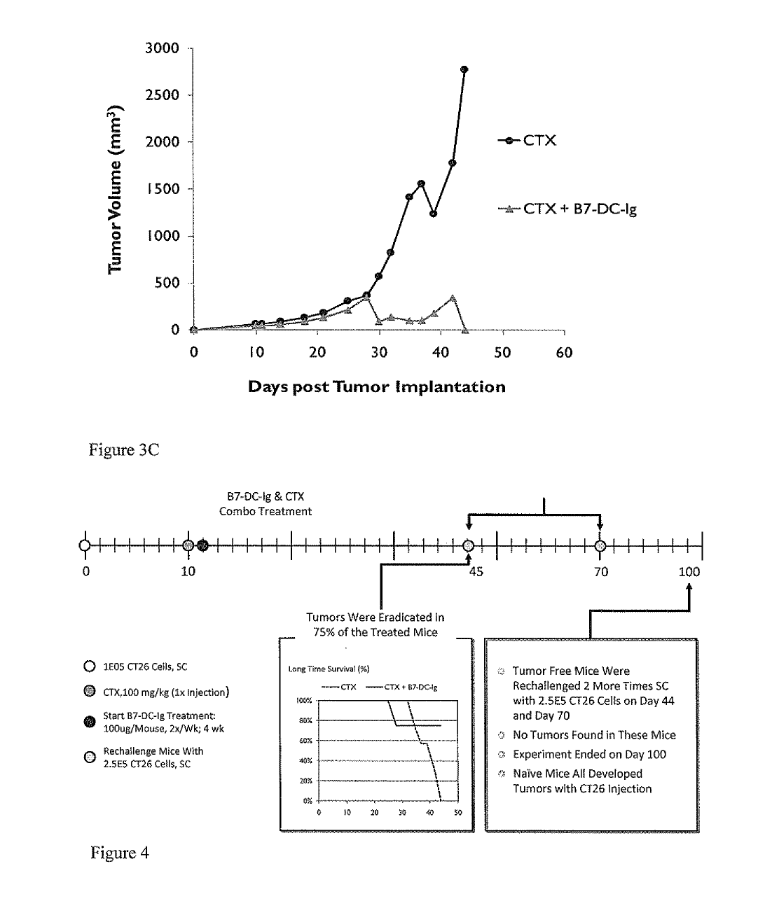

Balb/C mice at age of 9 to 11 weeks were implanted subcutaneously with 1.0×105 CT26 colorectal tumor cells. On day 10 post tumor implantation, mice received 100 mg/kg of cyclophosphamide. B7-DC-Ig treatment started 1 day later, on day 11. Mice were treated with 100 ug of B7-DC-Ig, 2 doses per week, for 4 weeks and total 8 doses. 75% of the mice that received the CTX+B7-DC-Ig treatment regimen eradicated the established tumors by Day 44, whereas all mice in the control CTX alone group died as a result of tumor growth or were euthanized because tumors exceeded the sizes approved by IACUC (results shown in FIG. 3). These results demonstrate the effectiveness of the treatment regimen on established tumors and not mere prophylaxis.

### Example 3

Combination of Cyclophosphamide and B7-DC-Ig can Eradicate Established Tumors and Protect Against Tumor Re-Challenge

Mice eradicated established CT26 colorectal tumors from the above described experiment were rechallenged with 1×105 CT26 cells on Day 44 and Day 70. No tumors grew out from the rechallenge suggesting they had developed long term anti-tumor immunity from the cyclophosphamide and B7-DC-Ig combination treatment. All mice in the vehicle control group developed tumors (results shown in FIG. 4). These results show the effectiveness of the treatment regimen on established tumors and that the cyclophosphamide and B7-DCIg combination treatment resulted in memory responses to tumor antigens.

### Example 4

Combination of Cyclophosphamide and B7-DC-Ig can Generate Tumor Specific, Memory Cytotoxic T Lymphocytes

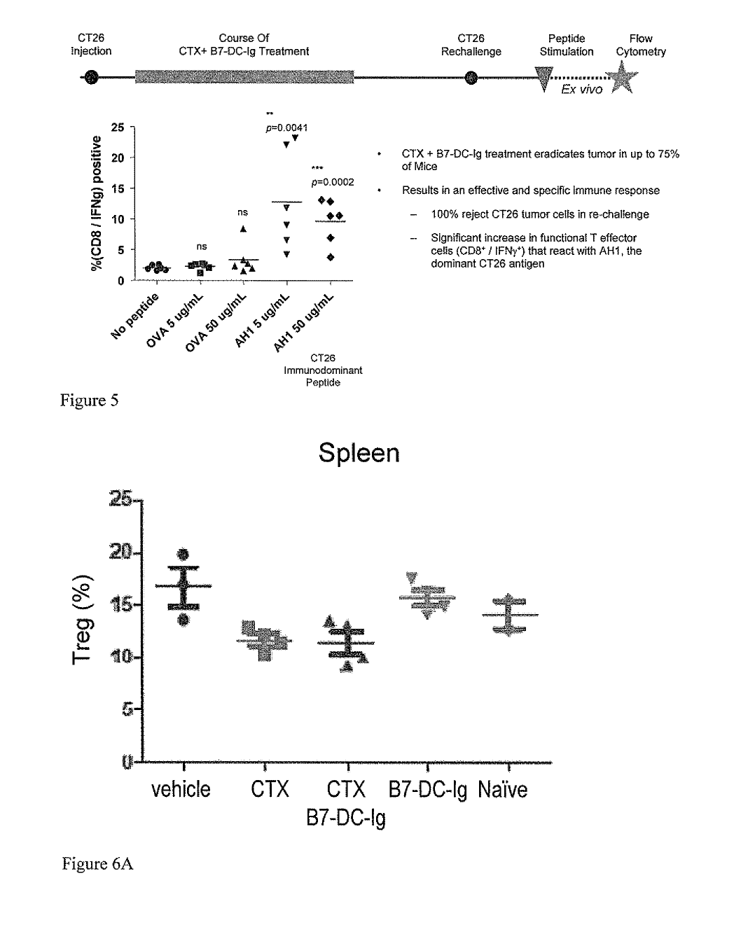

Mice eradiated established CT26 colorectal tumors from the above described experiment were rechallenged with 2.5×105 CT26 cells on Day 44. Seven days later, mouse spleens were isolated. Mouse splenocytes were pulsed with 5 or 50 ug/mL of ovalbumin (OVA) or AHI peptides for 6 hours in the presence of a Golgi blocker (BD BioScience). Memory T effector cells were analyzed by assessing CD8+/IFN□+ T cells. Results in FIG. 5 show that there were significant amount of CT26 specific T effector cells in the CT26 tumor-eradicated mice.

### Example 5

### Combination of Cyclophosphamide and B7-DC-Ig Regimen Leads to Reduction of Tregs in the Tumor Microenvironment

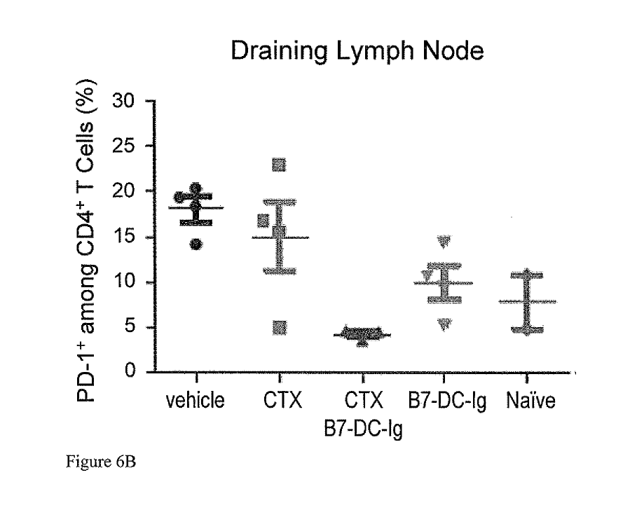

FIG. 6 shows the results of experiments wherein Balb/C mice at age of 9 to 11 weeks of age were implanted with 1×105 CT26 cells subcutaneously. On Day 9, mice were injected with 100 mg/kg of CTX, IP. Twenty four hours later, on Day 10, mice were treated with 100 ug of B7-DC-Ig. There were 5 groups: naïve mice that did not receive any tumor cells, vehicle injected, CTX alone, CTX+B7-DC-Ig or B7-DC-Ig alone. Two naïve mice and 4 mice from other groups were removed from the study on Day 11 (2 days post CTX) and Day 16 (7 days post CTX) for T cell analysis. Left panel shows on Day 11, 2 days post CTX injection, Treg in the spleen of the mice with CTX treatment was significantly lower than the one in the mice with tumor implantation and injected with vehicle. Right panel shows that on Day 16, 7 days post CTX and 6 days post B7-DC-Ig treatment, B7-DC-Ig significantly lowered the CD4+ T cells expressing high PD-1. This was observed in both the B7-DC-Ig treated and CTX+B7-DC-Ig treated mice. Mice implanted with tumor cells intended to have more PD-1+/CD4+ T cells in the draining LN compared with naïve mice.

### Example 6

### Combination of Cyclophosphamide and B7-DC-Ig can Promote Mouse Survival in a Metastatic Prostate Lung Tumor Model

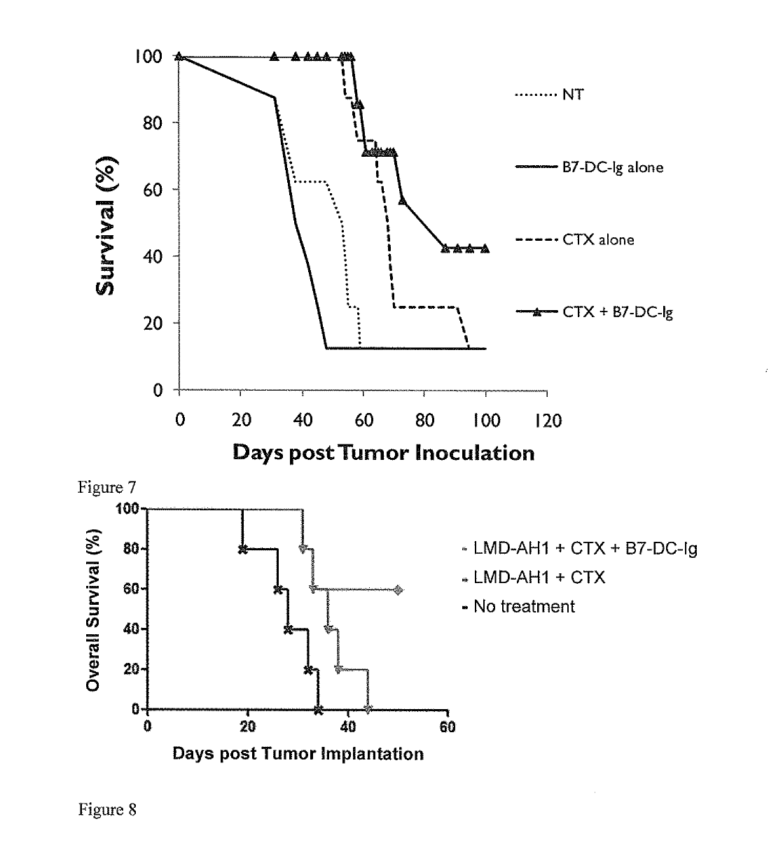

B10.D2 mice at age of 9 to 11 weeks were injected intravenously with 3.0×105 SP-1 mouse prostate tumor cells, which were isolated from lung metastasis post parent TRAMP prostate tumor cell injection. The CTX mice received 3 doses of CTX, 50 mg/kg, on Day 5, 12 and 19. The B7-DC-Ig treated mice received 3 doses of B7-DC-Ig, 5 mg/kg, on Day 6, 13 and 20. On Day 100, 17% of mice in the control groups, no-treated, CTX alone, B7-DC-Ig alone survived while 43% of the mice received combination of CTX and B7-DC-Ig survived. Results are shown in FIG. 7.

### Example 7

### Combination of Listeria Cancer Vaccine and B7-DC-Ig Can Enhance Mouse Survival Post CT26 Liver Implantation

Balb/C mice at age of 11-13 weeks were implanted with CT26 cells using a hemispleen injection technique (Yoshimura K et al., 2007, Cancer Research). On Day 10, mice received I injection of CTX at 50 mg/kg, IP. Twenty four hours later, on Day 11, mice were treated with recombinant Listeria carrying AH1 peptide, an immunodominant epitope of CT26, at 0.1 LD50 (1×10⁷ CFU), then on Day 14 and 17. Mice were also treated with B7-DC-Ig on Day 11 and then on Day 18. FIG. 8 shows mice without any treatment or treated with CTX and Listeria cancer vaccine all died before Day 45. There were 60% of the mice received triple combination, CTX+Listeria cancer vaccine and B7-DC-Ig survived.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A fusion protein comprising a first fusion partner comprising a T cell costimulatory polypeptide, or a fragment and/or variant thereof, fused (i) directly to a second fusion partner and, (ii) optionally fused to a linker peptide or polypeptide sequence that is fused to the second fusion partner,
wherein the costimulatory molecule or costimulatory fragment and/or variant thereof increases antigen-specific proliferation of T cells, enhances production of cytokines by T cells, stimulated differentiation or effector function of T cells, or promotes the survival of T cells, and
wherein the second fusion partner comprises a polypeptide that targets the fusion protein to cells of a tumor, tumor vasculature, or tissue involved in activation of an immune response.

2. The fusion protein of claim 1, wherein the costimulatory polypeptide comprises a B7 family costimulatory molecule or a fragment and/or variant thereof.

3. The fusion protein of claim 2, wherein the costimulatory molecule comprises a soluble fragment of a B7 family costimulatory molecule.

4. The fusion protein of claim 3, wherein the costimulatory molecule comprises the extracellular domain of a B7 family costimulatory molecule.

5. The fusion protein of claim 2, wherein the costimulatory molecule is selected from the group consisting of B7-DC, B7-1, B7-2, B7-H5, and fragments and/or variants thereof.

6. The fusion protein of claim 5, wherein the costimulatory molecule is a variant costimulatory molecule or fragment thereof,
wherein the costimulatory molecule or fragment thereof is a variant of a wild-type costimulatory molecule,
wherein the variant costimulatory molecule or fragment thereof comprises a substitution, deletion or insertion of one or more amino acids.

7. The fusion protein of claim 5, wherein the B7-DC polypeptide is murine B7-DC.

8. The fusion protein of claim 5, wherein the B7-DC polypeptide is human B7-DC.

9. The fusion protein of claim 5, wherein the B7-DC polypeptide is non-human primate B7-DC.

10. The fusion protein of claim 6, wherein the substitution, deletion or insertion of one or more amino acids is in the A′, B, C, C′, C″, D, E, F, or G strand of B7-DC, or any combination thereof.

11. The fusion protein of claim 1, wherein the second fusion partner comprises a polypeptide that binds to an antigen on a tumor or on tumor-associated neovasculature.

12. The fusion protein of claim 11, wherein the second fusion partner comprises a polypeptide that binds to a tumor-specific or a tumor-associated antigen.

13. The fusion protein of claim 12, wherein the tumor-specific or tumor-associated antigen is selected from the group consisting of alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F,5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

14. The fusion protein of claim 11, wherein the second fusion partner comprises a polypeptide that binds to an antigen that is specific to tumor-associated neovasculature or is more highly expressed in tumor neovasculature relative to normal vasculature.

15. The fusion protein of claim 14, wherein the antigen is selected from the group consisting of VEGF/KDR, Tie2, vascular cell adhesion molecule (VCAM), endoglin and α₅β₃ integrin/vitronectin.

16. The fusion protein of claim 1, wherein the second fusion partner comprises a chemokine or a chemokine receptor or a soluble fragment thereof.

17. The fusion protein of claim 16, wherein the second fusion partner comprises a soluble fragment of a chemokine receptor selected from the group consisting of CXCR2, CXCR4, CCR2 and CCR7, wherein the soluble fragment binds to a chemokine.

18. The fusion protein of claim 17, wherein the second fusion partner comprises a chemokine selected from the group consisting of CXC, CC, CX3C and C chemokines or a fragment thereof.

19. The fusion protein of claim 1, wherein the linker peptide or polypeptide comprises a flexible peptide or polypeptide, wherein the peptide or polypeptide comprises 2 or more amino acids, and
wherein the peptide or polypeptide comprises an amino acid sequence selected from the group consisting of Gly-Ser, Gly-Ser-Gly-Ser, Ala-Ser, Gly-Gly-Gly-Ser, (Gly₄-Ser)₃, (Gly₄-Ser)₄, and (Gly₄-Ser)₄.

20. The fusion protein of claim 1, wherein the linker peptide or polypeptide comprises the hinge region of a human immunoglobulin, and optionally, further comprises an additional region of an immunoglobulin selected from the group consisting of the Fc domain, the C_{H}1 region or the C_{L} region.

21. The fusion protein of claim 1, further comprising a domain that mediates dimerization or multimerization of the fusion protein to form homodimers, heterodimers, homomultimers, or heteromultimers.

22. The fusion protein of claim 21, wherein the domain that mediates dimerization or multimerization is selected from the group consisting of one or more cysteines that are capable of forming an intermolecular disulfide bond with a cysteine on the partner fusion protein, a coiled-coil domain, an acid patch, a zinc finger domain, a calcium hand domain, a C_{H}1 region, a C_{L} region, a leucine zipper domain, an SH2 (src homology 2) domain, an SH3 (src Homology 3) domain, a PTB (phosphotyrosine binding) domain, a WW domain, a PDZ domain, a 14-3-3 domain, a WD40 domain, an EH domain, a Lim domain, an isoleucine zipper domain, and a dimerization domain of a receptor dimer pair.

23. The fusion protein of claim 22, wherein the dimerization or multimerization domain is contained within the first fusion partner, the second fusion partner, or the linker peptide or polypeptide.

24. The fusion protein of claim 22, wherein the dimerization or multimerization domain is separate from and not contained within the first fusion partner, the second fusion partner, or the linker peptide or polypeptide.

25. A dimeric protein comprising a first and a second fusion protein, wherein the first and the second fusion proteins comprise the fusion protein of claim 1, wherein the first and the second fusion proteins are bound to one another by covalent or noncovalent bonds to form a dimer.

26. The dimeric protein of claim 25, wherein the dimer is a homodimer.

27. The dimeric protein of claim 25, wherein the dimer is a heterodimer.

28. A multimeric protein comprising more than two fusion proteins, wherein each of the fusion proteins comprise the fusion protein of claim 1, wherein the fusion proteins are bound to one another by covalent or noncovalent bonds to form a multimer.

29. The multimeric protein of claim 28, wherein the multimer is a homomultimer.

30. The multimeric protein of claim 29, wherein the multimer is a heteromultimer.

31. The dimeric or multimeric protein of claim 25, wherein the fusion proteins are bound together by disulfide bonds.

32. The dimeric or multimeric protein of claim 31 wherein the disulfide bonds are formed between cysteines in the linker peptide sequence.

33. An isolated nucleic acid molecule comprising a nucleic acid sequence that encodes the fusion protein of claim 1.

34. A vector comprising the nucleic acid of claim 33.

35. The vector of claim 34, wherein said nucleic acid sequence is operably linked to an expression control sequence.

36. A host cell comprising the vector of claim 35.

37. A pharmaceutical composition for use with an antigen or a vaccine to increase the immunogenicity of the antigen or vaccine comprising:
a) the isolated fusion protein, dimeric protein, or multimeric protein of claim 1, and
b) a pharmaceutically and immunologically acceptable excipient or carrier.

38. An immunogenic composition useful for inducing a T cell immune response against a tumor, comprising
(a) a source of antigen to which an immune response is desired;
(b) a fusion protein, dimeric protein, or multimeric protein of claim 1,
(c) optionally, a general immunostimulatory agent or adjuvant; and
(d) a pharmaceutically and immunologically acceptable excipient or carrier for (a), (b) and, optionally, (c).

39. A method for costimulating T cells comprising contacting a T cell with the fusion protein, dimeric protein, or multimeric polypeptide of claim 1.

40. The method of claim 39, wherein the method comprises administering the fusion protein to a mammal.

41. A method for increasing the activation of tumor-infiltrating T cells in a subject by administering to a mammal in need thereof an effective amount of a fusion protein, dimeric protein, or multimeric protein of claim 1, or a nucleic acid encoding the same, to activate the mammal's T cells.

42. A method for increasing the population of tumor-infiltrating T cells in a subject by administering to a mammal in need thereof an effective amount of a fusion protein, dimeric protein, or multimeric protein of claim 1, or a nucleic acid encoding the same, to activate the mammal's T cells.

43. A method for stimulating or augmenting an effective anti-tumor T cell response by administering to a mammal in need thereof an effective amount of a fusion protein, dimeric protein, or multimeric protein of claim 1, or a nucleic acid encoding the same, to activate the mammal's T cells.

44. A method for potentiating an immune response to an antigen or a vaccine in a mammalian subject, comprising administering to the mammal, in combination with the antigen or vaccine, the fusion protein, dimeric protein, or multimeric protein of claim 1, or a nucleic acid encoding the same, in an effective amount to activate the subject's T cells.
